(19) United States
(12) Patent Application Publication
(10) Pub. No.: US 2026/0198775 A1
(43) Pub. Date: Jul. 16, 2026

(54) Methods and Devices for Multiphoton Imaging and Laser-Tissue Interactions

(71) Applicant: The Regents of the University of California, Oakland, CA (US)

(72) Inventors: Thomas Gentry, San Francisco, CA (US); Ryan Morton, San Francisco, CA (US); Tyson Kim, San Francisco, CA (US)

(21) Appl. No.: 19/134,180

(22) PCT Filed: Dec. 8, 2023
(86) PCT No.: PCT/US2023/083133

§ 371 Date: May 29, 2025

Related U.S. Application Data
(60) Provisional application No. 63/431,428, filed on Dec. 9, 2022.

(51) Int. Cl. A61B 3/12; A61B 3/00; A61B 5/00; A61F 9/008

(52) U.S. Cl. CPC A61B 3/1241; A61B 3/0008; A61B 3/0025; A61B 5/0071; A61B 5/4836; A61F 9/00821; A61F 9/00825; A61F 2009/00891

(57) ABSTRACT

Methods, systems and adaptors for multiphoton imaging of, and laser-tissue interactions with, non-transparent tissue, such as ocular and periocular tissue, are provided. Aspects of the present invention include methods of imaging a structure through non-transparent tissue, such as ocular or periocular tissue, comprising: deploying an excitation source to transmit light energy to a structure through non-transparent tissue, such as ocular or periocular tissue, detecting light emitted from the structure via multiphoton excitation through the non-transparent tissue, such as ocular or periocular tissue, and imaging the structure based on the detected light. Aspects of the present invention further include methods of treating tissue, such as ocular or periocular tissue or dermatologic tissue, by manipulating an imaged structure, including, for example, providing non-incisional therapy, photo-tissue interactions, multi-photon-mediated damage, such as thermal damage, photo-disruption, photo cross-linking, blood vessel coagulation or ablating tissue. Aspects of the present invention further include methods of imaging dermatologic tissues or providing dermatologic treatments. Aspects of the present invention further include methods of providing cosmetic treatments. Aspects of the present invention further include methods of guiding delivery of gene therapy or cellular therapy in ocular or periocular tissue. Also provided are systems and adaptors for performing the methods described herein.

### CROSS-REFERENCE TO RELATED APPLICATION

Pursuant to 35 U.S.C. § 119(e), this application claims priority to the filing date of United States provisional patent application Ser. No. 63/431,428 filed Dec. 9, 2022, the disclosure of which application is incorporated herein by reference in its entirety.

### INTRODUCTION

Techniques for imaging, as well as manipulating, tissue, such as ocular and periocular tissue, offer a means of visualizing structures, such as ocular structures, studying photoreceptors and their function as well as studying and altering the dynamics of biology and disease, such as ocular biology and disease. However, existing approaches for imaging and manipulating tissue, such as ocular and periocular tissue, have limited resolution and are unable to image deeper structures or peripheral structures such as, for example, the choroid, peripheral retina or ciliary body. This is due to various limitations, such as intrinsic constraints of transpupillary imaging as well as the high absorptivity and scattering of uveoscleral and periocular tissues. Certain optical tools for intraocular imaging and therapy rely exclusively on a transpupillary approach or require incisional surgery in the operating rooms.

In addition, existing dermatologic or cosmetic treatments, in particular laser-based dermatologic or cosmetic treatments, are limited due to existing optical techniques. Such existing techniques may be limited respect to the depth at which tissues can be manipulated, the accuracy available for manipulating tissues or may be limited insofar as they are only capable of delivering thermal-based treatments and not other types of treatments for manipulating tissues.

### SUMMARY

Thus, there is a need for improved and useful methods and systems for imaging, as well as manipulating, tissues, such as ocular and periocular tissue, including a need for improved techniques for delivering dermatologic and cosmetic treatments or therapies. In particular, there is a need for imaging, as well as manipulating, deeper into tissue, such as ocular and periocular tissue, through non-transparent tissue, than what is available through existing techniques. There is also a need for manipulating tissue, including deeper into tissue, using techniques other than thermal-based treatments, such as providing or applying non-incisional therapy, photo-tissue interactions, laser-tissue interactions, laser-tissue perturbations, photo-disruption, blood vessel coagulation, ablation or the like. There is also a need for delivering thermal-based treatments with greater accuracy and greater precision than existing thermal-based treatments, such as applying multi-photon-mediated thermal damage. This invention provides such new and useful methods and systems, addressing the limitations mentioned above. To accomplish this, the invention utilizes multiphoton microscopy, including next generation techniques, such as three-photon excited fluorescence (3PEF) microscopy, second Harmonic Generation (2HG) and third-harmonic generation (3HG), in order to image structures, as well as multiphoton tissue interactions to manipulate structures, in each case through non-transparent tissue, such as ocular or periocular tissue, thereby accessing deeper structures and peripheral structures that were previously inaccessible using existing techniques or with more accuracy or with finer granularity than existing techniques.

Embodiments of the present invention are capable of visualizing any convenient structure, such as ocular and periocular structures, including anterior and superficial structures as well as deep and peripheral structures, such as ocular structures, such as the chorioretinal vasculature, retinal pigmented epithelium and ciliary body, in each case with elegant cellular detail. To the knowledge of the inventors, this invention is the first to successfully employ such optical techniques to visualize peripheral ocular structures in the living eye, such as, for example, the ciliary body, and to provide such high resolution of deeper ocular structures. Unlike existing approaches, embodiments of this invention need not rely on a transpupillary approach. Embodiments of the present invention provide an imaging tool for visualizing structures, such as ocular structures, with high resolution, including intraocular structures previously inaccessible by optical approaches including the ciliary body, peripheral retina and choroid and can do so with cellular resolution and functional imaging capabilities. In addition, embodiments of the present invention provide a tool for studying cellular and molecular biology in the living eye and periorbita. Embodiments of the present invention provide techniques for aiding diagnosis of disease, such as aiding diagnosis of cancerous tissues. Embodiments of the present invention provide techniques for treatment of certain conditions, such as breaking up coagulated fluids blocking tear ducts and causing symptoms of dry eye. Embodiments of the present invention provide techniques for dermatologic applications, such as performing a biopsy procedure or other surgeries or treatment of scars. Embodiments of the present invention provide techniques for cosmetic applications, such as manipulating the shape or volume of fat deposits, such as fat deposits surrounding the eye.

Background information, including background information regarding aspects of two-photon microscopy, is provided in: Helmchen, F. and W. Denk, Deep tissue two-photon microscopy. Nat Methods, 2005. 2(12): p. 932-40, as well as Zipfel, W. R., R. M. Williams, and W. W. Webb, Nonlinear magic: multiphoton microscopy in the biosciences. Nat Biotechnol, 2003. 21(11): p. 1369-77, the disclosures of which are incorporated herein in their entireties. In general, the depth of multiphoton imaging is intrinsically limited to approximately five times the attenuation distance of an excitation wavelength within a tissue, and imaging through highly scattering and absorptive tissues (e.g., bone or sclera) is a challenging task. As such, 3PEF imaging and other higher order techniques enable imaging, as well as manipulation of, far deeper tissues. Background information, including background information regarding aspects of three-photon microscopy, is provided in: Wang T, et al. Three-photon imaging of mouse brain structure and function through the intact skull. Nat Methods. 2018 Oct; 15(10):789-792. doi: 10.1038/s41592-018-0115-y. Epub 2018 Sep. 10. PMID: 30202059; PMCID: PMC6188644, and Cheng Y T, Lett K M, Schaffer C B. Surgical preparations, labeling strategies, and optical techniques for cell-resolved, in vivo imaging in the mouse spinal cord. Exp Neurol. 2019 August 318:192-204. doi: 10.1016/j.expneurol.2019.05.010. Epub 2019 May 13. PMID: 31095935; PMCID: PMC6588420, the disclosures of which are incorporated herein in their entireties. Background information, including background information regarding imaging results obtained using second harmonic generation, is provided in: Campagnola P J, Loew L M. Second-harmonic imaging microscopy for visualizing biomolecular arrays in cells, tissues and organisms. Nat Biotechnol. 2003 Nov; 21(11):1356-60. doi: 10.1038/nbt 894. PMID: 14595363.

In addition, embodiments of the present invention provide a non-incisional therapy tool for use in a variety of applications, previously impossible or limited to incisional therapy, such as, for example: (i) for precise and cellularly-targeted photocoagulation and/or tissue disruption of the ciliary body to controllably and safely reduce aqueous production of the eye for the treatment of glaucoma; (ii) for performing transscleral laser trabeculotomy and/or trabeculoplasty and/or sclerotomy to increase outflow of aqueous humor through conventional drainage pathways for the treatment of glaucoma; (iii) for identifying and providing photocoagulation therapy to peripheral retinal tears or irregularities for the treatment and prevention of retinal detachment; (v) for identifying and providing photocoagulation and thermal treatment to ciliary body tumors and peripheral choroidal tumors; (vii) for photo-crosslinking of scleral tissue for prevention of myopia; (viii) for identifying and performing targeted alteration of extraocular muscle function; (ix) and visualization and targeted thermal and photocoagulation therapy of the orbital fat; (x) dermatologic applications, such as imaging dermal tissue, distinguishing cancerous versus non-cancerous tissues, diagnosing melanoma, ablating or otherwise disrupting cancerous tissues, such as melanomas, performing skin biopsy surgeries or other dermatologic procedures and application; or (xi) cosmetic applications, such as affecting the shape or volume of fat deposits within tissue, affecting the appearance of skin, e.g., skin tightening, or the color of skin, e.g., removal of discolorations or scars or tattoos, or other cosmetic applications. Embodiments of the present invention provide a tool for use in drug or cell or gene delivery by enabling high resolution imaging of previously inaccessible structures for precise localization (e.g., within ocular or periocular tissue) for delivery of such drug or cell or gene or the like.

As described, optical imaging in the eye is critical for ophthalmic care and is also a powerful approach for assessing systemic health and disease. For example, capturing images indicative of subtle changes in the neurovascular retina can be analyzed to provide accurate insight into both ocular and systemic conditions ranging from cardiovascular health to metabolic disorder. However, conventional methods for imaging through the pupil offer only a limited view of the retina, and many critical intraocular structures are shrouded by opaque and heavily pigmented tissues. For example, conventional optical imaging offers only a limited view of the retina, and the majority of intraocular tissues, such as the ciliary body, choroid, peripheral retina, and peripheral retinal pigment epithelium (RPE) are beyond reach or inadequately visualized. These concealed regions play pivotal roles in the development and progression of ocular diseases e.g., the ciliary body produces aqueous humor of the eye and critically regulates glaucoma pathophysiology, and the choroid is a highly vascularized tissue that supports photoreceptors, the RPE, and provides immune surveillance in the eye, and changes in the choroid can reflect systemic conditions including autoimmune diseases, metastatic cancers, systemic infections, hematologic disorders, hypertension, and diabetes. Embodiments of the present invention utilizing multiphoton microscopy are capable of deep imaging with subcellular resolution, including with respect to such regions. Embodiments of the present invention further utilizing adaptive optics and laser technology enable higher-order nonlinear processes such as three-photon excited fluorescence and third-harmonic generation to be produced further into highly scattering tissues, including such regions, or, for example, directly through mouse skull or directly through the opaque scleral wall of the eye.

Embodiments comprising laser sources configured for higher-order nonlinear processes such as three-photon excited fluorescence (3PEF) and third harmonic generation (THG) allow far deeper imaging. Their longer excitation wavelengths have dramatically less scattering and phototoxicity, resulting in greater penetration and imaging through the intact skull. Embodiments utilizing 3PEF provide excellent vascular imaging when combined with intravenous injection of fluorescent dyes, and embodiments utilizing THG provides label-free contrast of tissues including blood vessels. In ocular tissue, embodiments utilizing THG can be configured to visualize the inner nuclear layer, outer nuclear layer, ganglion cell layer, and retinal pigmented epithelium. See Rim, T. H., et al., Prediction of systemic biomarkers from retinal photographs: development and validation of deep-learning algorithms. Lancet Digit Health, 2020. 2(10): p. e 526-e536; Wagner, S. K., et al., Insights into Systemic Disease through Retinal Imaging-Based Oculomics. Transl Vis Sci Technol, 2020. 9(2): p. 6; Poplin, R., et al., Prediction of cardiovascular risk factors from retinal fundus photographs via deep learning. Nat Biomed Eng, 2018. 2(3): p. 158-164; Anand, N., et al., A Review of Cyclodestructive Procedures for the Treatment of Glaucoma. Semin Ophthalmol, 2020. 35(5-6): p. 261-275; Kiel, J. W. and H. A. Reitsamer, Relationship between ciliary blood flow and aqueous production: does it play a role in glaucoma therapy? J Glaucoma, 2006. 15(2): p. 172-81; Nickla, D. L. and J. Wallman, The multifunctional choroid. Prog Retin Eye Res, 2010. 29(2): p. 144-68; Kongwattananon, W., T. Pothikamjorn, and T. Somkijrungroj, Posterior segment manifestations of ocular metastasis. Curr Opin Ophthalmol, 2023; Nowinska, A.K., et al., Ocular Manifestations of Systemic Diseases. J Ophthalmol, 2018. 2018: p. 7851691; Helmchen, F. and W. Denk, Deep tissue two-photon microscopy. Nat Methods, 2005. 2(12): p. 932-40; Zipfel, W. R., R. M. Williams, and W. W. Webb, Nonlinear magic: multiphoton microscopy in the biosciences. Nat Biotechnol, 2003. 21(11): p. 1369-77; Wang, T., et al., Three-photon imaging of mouse brain structure and function through the intact skull. Nat Methods, 2018. 15(10): p. 789-792; Ouzounov, D. G., et al., In vivo three-photon imaging of activity of GCaMP6-labeled neurons deep in intact mouse brain. Nat Methods, 2017. 14(4): p. 388-390; Yildirim, M., et al., Functional imaging of visual cortical layers and subplate in awake mice with optimized three-photon microscopy. Nat Commun, 2019. 10(1): p. 177; Masihzadeh, O., et al., Third harmonic generation microscopy of a mouse retina. Mol Vis, 2015. 21: p. 538-47; Wu, J., et al., Kilohertz two-photon fluorescence microscopy imaging of neural activity in vivo. Nat Methods, 2020. 17(3): p. 287-290; Kim, T. N., et al., Line-scanning particle image velocimetry: an optical approach for quantifying a wide range of blood flow speeds in live animals. PLoS One, 2012. 7(6): p. e38590; Briers, J. D., Laser Doppler, speckle and related techniques for blood perfusion mapping and imaging. Physiol Meas, 2001. 22(4): p. R35-66; Hu, S. and L. V. Wang, Photoacoustic imaging and characterization of the microvasculature. J Biomed Opt, 2010. 15(1): p. 011101; Meng, G., et al., Ultrafast two-photon fluorescence imaging of cerebral blood circulation in the mouse brain in vivo. Proc Natl Acad Sci U S A, 2022. 119(23): p. e2117346119; Ji, N., Adaptive optical fluorescence microscopy. Nat Methods, 2017. 14(4): p. 374-380; Zhang, Q., et al., Adaptive optics for optical microscopy [Invited]. Biomed Opt Express, 2023. 14(4): p. 1732-1756; Wang, K., et al., Direct wavefront sensing for high-resolution in vivo imaging in scattering tissue. Nat Commun, 2015. 6: p. 7276; Rodriguez, C., et al., An adaptive optics module for deep tissue multiphoton imaging in vivo. Nat Methods, 2021. 18(10): p. 1259-1264; Wang, C., et al., Multiplexed aberration measurement for deep tissue imaging in vivo. Nat Methods, 2014. 11(10): p. 1037-40; Jung, S., et al., Analysis of fractalkine receptor CX(3)CR1 function by targeted deletion and green fluorescent protein reporter gene insertion. Mol Cell Biol, 2000. 20(11): p. 4106-14; Kang I, Z. Q., Yu S X, and Ji N, Coordinate-based neural representations for computational adaptive optics in widefield microscopy. bioRxiv, 2023, incorporated by reference herein.

Methods, systems and adaptors for multiphoton imaging of, and laser-tissue interactions with, non-transparent tissue, such as ocular and periocular tissue, are provided. Aspects of the present invention include methods of imaging a structure through non-transparent tissue, such as ocular or periocular tissue, comprising: deploying an excitation source to transmit light energy to a structure through non-transparent tissue, such as ocular or periocular tissue, detecting light emitted from the structure via multiphoton excitation through the non-transparent tissue, such as ocular or periocular tissue, and imaging the structure based on the detected light. Aspects of the present invention further include methods of treating tissue, such as ocular or periocular tissue or dermatologic tissue, by manipulating an imaged structure, including, for example, providing non-incisional therapy, photo-tissue interactions, multi-photon-mediated damage, such as thermal damage, photo-disruption, photo cross-linking, blood vessel coagulation or ablating tissue. Aspects of the present invention further include methods of imaging dermatologic tissues or providing dermatologic treatments. Aspects of the present invention further include methods of providing cosmetic treatments. Aspects of the present invention further include methods of guiding delivery of gene therapy or cellular therapy in ocular or periocular tissue. Also provided are systems and adaptors for performing the methods described herein.

### BRIEF DESCRIPTION OF THE FIGURES

The invention may be best understood from the following detailed description when read in conjunction with the accompanying drawings. Included in the drawings are the following figures:

FIGS. 1A-E depict flow diagrams of methods for imaging a structure through non-transparent tissue, such as ocular or periocular tissue, as well as utilizing imaging results to conduct flow analysis, in each case according to embodiments of the present invention.

FIGS. 2A-N depict embodiments of adaptors for coupling an optical system to non-transparent ocular tissue as well as aspects related to immersion media and gels used in connection with embodiments of adaptors, in each case according to the present invention.

FIG. 3 depicts a schematic view of an exemplary system for imaging a structure through non-transparent ocular or periocular tissue according to an embodiment of the present invention.

FIGS. 4A-B depict exemplary systems for imaging a structure through non-transparent ocular or periocular tissue according to embodiments of the present invention.

FIGS. 5A-F depict imaging results of 3PEF transscleral imaging to visualize the chorioretinal vasculature and chorioretinal anastomoses.

FIG. 6 depicts imaging results of third-harmonic generation (THG) transscleral imaging to visualize retinal pigmented epithelium.

FIGS. 7A-B depict imaging results of THG transscleral imaging of the ciliary body.

FIGS. 8A-E depict imaging results of imaging intravital cellular imaging in the living eye.

FIG. 9 depicts an example of imaging chorioretinal vascular dysgenesis.

FIGS. 10A-F depict imaging results of choroidal blood flow and remodeling.

FIG. 11 depicts an exemplary quantitative analytical technique for blood flow analysis based on imaging data collected according to an embodiment of the present invention.

FIGS. 12A-D depict hemodynamic analysis with line-scanning particle image velocimetry.

FIGS. 13A-C depict aspects of an ophthalmic imaging and therapeutic application of embodiments of the present invention.

FIGS. 14A-C present an outline of another ophthalmic imaging and therapeutic application of embodiments of the present invention.

FIGS. 15A-B present schematics of another ophthalmic imaging and therapeutic application of embodiments of the present invention.

FIG. 16 depicts an overview of an arrangement of elements of embodiment of the present invention for use imaging ocular tissue in a clinical setting.

FIG. 17 depicts an overview of a potential structure for use with embodiments of the present invention in connection imaging ocular tissue.

FIG. 18 depicts aspects of three-photon excited fluorescence (3PEF) in comparison with two-photon excited fluorescence (2PEF).

FIGS. 19A-B present an overview of another ophthalmic imaging and therapeutic application of embodiments of the present invention.

### DETAILED DESCRIPTION

Methods, systems and adaptors for multiphoton imaging of, and laser-tissue interactions with, non-transparent tissue, such as ocular and periocular tissue, are provided. Aspects of the present invention include methods of imaging a structure through non-transparent tissue, such as ocular or periocular tissue, comprising: deploying an excitation source to transmit light energy to a structure through non-transparent tissue, such as ocular or periocular tissue, detecting light emitted from the structure via multiphoton excitation through the non-transparent tissue, such as ocular or periocular tissue, and imaging the structure based on the detected light. Aspects of the present invention further include methods of treating tissue, such as ocular or periocular tissue or dermatologic tissue, by manipulating an imaged structure, including, for example, providing non-incisional therapy, photo-tissue interactions, multi-photon-mediated damage, such as thermal damage, photo-disruption, photo cross-linking, blood vessel coagulation or ablating tissue. Aspects of the present invention further include methods of imaging dermatologic tissues or providing dermatologic treatments. Aspects of the present invention further include methods of providing cosmetic treatments. Aspects of the present invention further include methods of providing gene therapy in ocular or periocular tissue, i.e., guiding delivery of gene therapy. Also provided are systems and adaptors for performing the methods described herein.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention.

The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method may be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods for Imaging, as Well as Manipulating, a Structure Through Non-Transparent Tissue

Aspects of the present disclosure include methods for imaging, as well as manipulating, a structure through non-transparent tissue, such as ocular or periocular tissue. In particular, the present disclosure includes methods of imaging a structure through non-transparent tissue, such as ocular or periocular tissue, comprising: deploying an excitation source to transmit light energy to a structure through non-transparent tissue, such as ocular or periocular tissue, detecting light emitted from the structure via multiphoton excitation through the non-transparent tissue, such as ocular or periocular tissue, and imaging the structure based on the detected light.

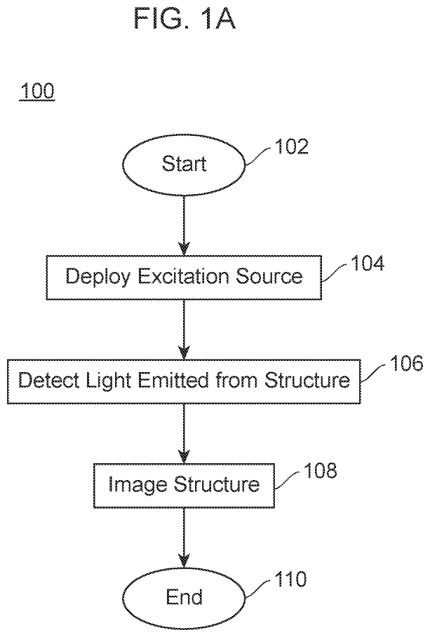

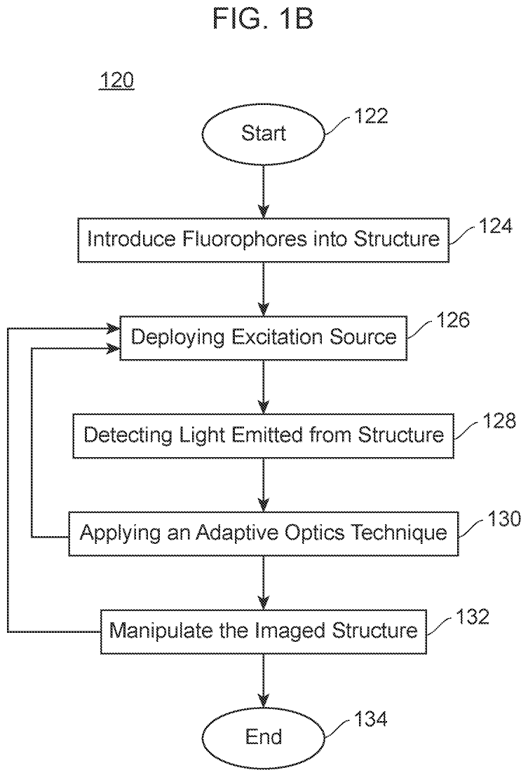

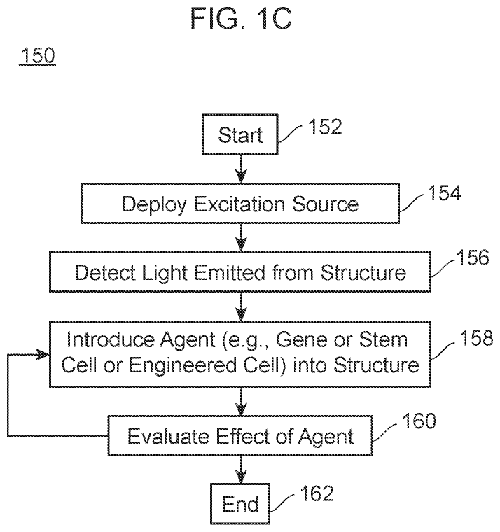

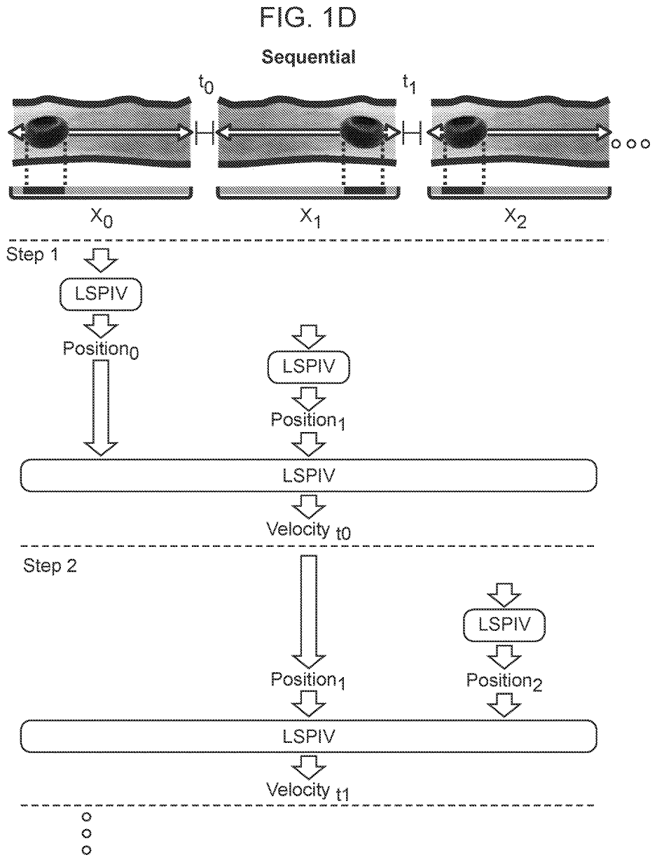

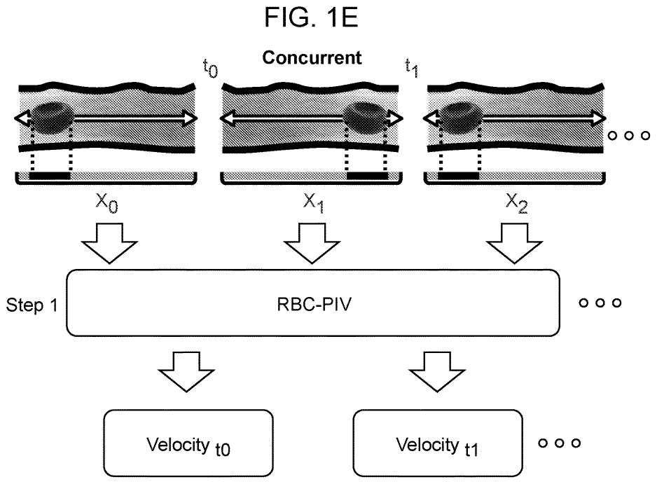

FIG. 1A illustrates a flow diagram 100 for imaging a structure through non-transparent tissue, such as ocular or periocular tissue, according to an embodiment of the present invention. The embodiment of the present invention depicted in FIG. 1A relates to imaging any convenient structure accessible through non-transparent tissue, such as ocular or periocular tissue, according to the present techniques and such may vary. Flow diagram 100 is an exemplary embodiment of the present invention provided for illustrative purposes, and the structure as well as the optical technique applied may vary as desired in embodiments of the present invention.

Flow diagram 100 starts at step 102. From starting step 102, the process proceeds next to step 104.

At step 104, an excitation source is deployed. By deploying an excitation source, it is meant that the excitation source is operably interfaced with the non-transparent tissue, such as ocular or periocular tissue, through which the structure is to be visualized. That is, the excitation source may be positioned in any convenient manner relative to the non-transparent tissue, such as ocular or periocular tissue, such that light emitted from the excitation source is transmitted through the non-transparent tissue, such as ocular or periocular tissue. In embodiments, the excitation source may be positioned on a surface of, or proximal to, the non-transparent tissue, such as ocular or periocular tissue. The operable interface between the excitation source and the non-transparent tissue, such as ocular or periocular tissue, may be located, for example, on a surface of an eye, such as on scleral tissue or partly on scleral tissue or partly on a cornea, or on a surface of periocular tissue, such as over one or more fat pads below an eye or any other convenient location enabling the excitation source to transmit light energy to a structure through non-transparent tissue, such as ocular or periocular tissue. Such location may be determined, for example, by a clinician taking into consideration anatomical or physiological constraints allowing access to the structure intended to be imaged according to the present invention.

### Non-transparent Tissue:

With respect to the non-transparent tissue, such as ocular or periocular tissue, in embodiments, the non-transparent tissue, such as ocular or periocular tissue, may comprise any tissue, through which light energy may be transmitted to the structure intended to be imaged according to the present invention, and such may vary. Non-transparent ocular or periocular tissue may comprise exclusively ocular tissue, exclusively periocular tissue, other tissues, such as dermatologic tissues, or combinations thereof. In embodiments, non-transparent ocular tissue comprises one or more of: scleral tissue, retinal pigment epithelium (RPE), uvea, conjunctiva, Tenon's capsule, ocular muscles or ciliary body, or tissues or structures proximal thereto. In other embodiments, non-transparent periocular tissue comprises one or more of: palpebral conjunctiva, orbital septum, capsolupalpebral fascia, tarsus, tarsal glands, periocular adipose tissue or dermis, or tissues or structures proximal thereto. In embodiments, non-transparent tissue comprises dermatologic tissue, such as epidermis, dermis or hypodermis.

In embodiments, the non-transparent tissue, such as ocular or periocular tissue, comprises light-scattering tissue. By light-scattering, it is meant that light striking the non-transparent tissue results in light radiating in different directions other than only its incident direction. In other embodiments, the non-transparent tissue, such as ocular or periocular tissue, comprises light-absorbing tissue. In still other embodiments, the non-transparent tissue, such as ocular or periocular tissue, comprises pigmented uveal tissues. In other embodiments, by non-transparent tissue, it is meant tissue that is non-transparent to typical wavelengths in the visual spectrum.

### Imaged Structure:

With respect to the imaged structure, i.e., the structure intended to be imaged through the non-transparent tissue, such as ocular or periocular tissue, in embodiments, the imaged structure comprises any convenient structure accessible through non-transparent tissue, such as ocular or periocular tissue, and such may vary. In embodiments, the imaged structure comprises one or more of: scleral tissue, corneal tissue, ocular vasculature, suprachoroidal space, choroid, chorioretinal vasculature, choriocapillaris, retinal pigment epithelium (RPE), photoreceptors, uvea, conjunctiva, Tenon's capsule, ocular muscles, ciliary body or peripheral retina, or tissues or structures proximal thereto. In other embodiments, the imaged structure comprises one or more of: palpebral conjunctiva, orbital septum, capsolupalpebral fascia, tarsus, tarsal glands, periocular adipose tissue or dermis, or tissues or structures proximal thereto. In still other embodiments, the imaged structure comprises capillaries, venules, veins, arterioles or arteries of the choroid, or tissues or structures, such as other vasculature, proximal thereto. In other embodiments, the imaged structure comprises fat deposits, nerve structures or pain receptors or glands such as tear ducts.

The imaged structure may be a dynamic structure; i.e., the imaged structure may comprise an image or series of images depicting movement or changes in a structure. In embodiments, the imaged structure comprises circulating cells. In embodiments, the imaged structure comprises fluid flow within tissue.

In embodiments, the imaged structure is present in front of a retinal pigment epithelium (RPE). In other embodiments, the imaged structure is present behind a retinal pigment epithelium (RPE).

In some cases, the imaged structure comprises light-scattering tissue or light-absorbing tissue or combinations thereof.

In certain embodiments, the method further comprises introducing fluorescent contrast agent or tag into the imaged structure or applying other labeling techniques to the imaged structure. In some cases, the method further comprises labeling blood plasma present in the imaged structure with a fluorescent dye. Any convenient fluorescent dye may be applied, such as a dye comprising particles, such as fluorophores, that emits stimulated light in response to receiving light transmitted from the excitation source. Any convenient commercially available fluorophores may be applied, and such may vary. Fluorophores of interest include, for example, Fluorescein, TexasRed, Alexa 680, quantum dots or the like. Fluorophores may be tagged to dextrans, antibodies, cells, drugs and molecular agents or the like. These fluorescently-tagged agents may be introduced into the blood plasma through intravenous injection. In embodiments, longer excitation wavelengths, i.e., transmitted by an excitation source, can induce higher-order nonlinear processes to excite traditional fluorophores (e.g., excitation wavelength of 1.3 μm can three-photon pump green fluorescent molecules such as GFP and FITC, while 1.7 μm light efficiently pumps red fluorescent molecules such as RFP and TexasRed).

By deploying an excitation source, as in step 104, it is further meant that the excitation source is activated, i.e., turned on, such that the excitation source emits energy, such as radiant energy, such as 2light energy. In embodiments, the excitation source is deployed to transmit light energy through the non-transparent tissue, such as ocular or periocular tissue, to the structure intended to be imaged.

### Excitation Source:

With respect to the excitation source as well as the light energy, i.e., radiant energy, transmitted therefrom, the excitation source may comprise one or more sources of light energy. Any convenient excitation source or sources, capable of transmitting sufficient light energy through the non-transparent tissue, such as ocular or periocular tissue, may be applied, and such may vary. By sufficient light energy, it is meant light energy, first, capable of being transmitted through the non-transparent tissue, such as ocular or periocular tissue, and, second, capable of causing light to be emitted from the structure via multiphoton excitation. Moreover, the light energy transmitted to the structure through the non-transparent tissue, such as ocular or periocular tissue, must be sufficient to cause adequate light to be emitted from the structure via multiphoton excitation that the emitted light is itself capable of detection through the non-transparent tissue, such as ocular or periocular tissue. Examples of multiphoton excitation of interest include, for example, multiphoton excitation involving excitation of a molecule resulting in emitting/stimulating light energy via two or more photons, such as, for example, two-photon excited fluorescence (2PEF), second harmonic generation (SHG), three-photon excited fluorescence (3PEF), third harmonic generation (THG), four-photon excited fluorescence, fourth harmonic generation or other higher order multiphoton processes.

In embodiments, the excitation source comprises one or more laser systems. Any convenient laser, capable of transmitting sufficient light energy and intensity at the appropriate wavelength through the non-transparent tissue, such as ocular or periocular tissue, may be applied, and such may vary. In some embodiments, the laser system includes a solid-state laser. In other embodiments, the laser system includes a fiber laser. In still other embodiments, the laser system includes a ytterbium-doped fiber laser. The laser system may be configured to generate light energy having any convenient wavelength and/or pulse duration and/or power output, and such may vary. In embodiments, the laser system is configured to generate light energy having a wavelength ranging from 350 nm to 5.0 μm, pulse duration of 1 to 1,000 femtoseconds, maximum power output of greater than 100 Watts, and a pulse repetition ranging between 1 Hz to 1,000 MHz. In certain contexts, a useful range of wavelengths of light energy generated by the laser system is between approximately 600 to 1,700 nm. In embodiments, per pulse energy may vary depending on the pulse rate of the laser. Higher average power (i.e., wattage) is a more broadly applicable metric with respect to lasers of interest. Lasers capable or emitting higher power are desirable since such emitted power can be attenuated to any desired level. Higher power may also be desirable to allow faster pulse rates, i.e., dividing up the wattage of emitted light energy into per pulse energy. Lasers capable of faster pulse rates (with sufficient energy) allow faster imaging and/or laser treatment. In other embodiments, the laser system includes dispersion compensation. By dispersion compensation, it is meant, applying any convenient technique, such as applying optical elements, to control or cancel or compensate the chromatic dispersion of the light energy emitted by the laser system and/or generated by the optical system and tissue downstream of the laser system.

Dispersion compensation may be achieved by, for example, manually applying optical components internal or external to the laser. Further details regarding laser systems of interest are described: RP Photonics Encyclopedia: Femtosecond Lasers, https://www.rp-photonics.com/femtosecond_lasers.html (last visited Oct. 25, 2022), the disclosure of which is incorporated herein in its entirety. Other known or yet to be discovered or implemented excitation sources or lasers or laser systems or optical systems may be applied as desired.

In embodiments, the excitation source comprises a first laser. Any convenient laser, capable of transmitting sufficient light energy at the appropriate wavelength through non-transparent tissue, such as ocular or periocular tissue, may be applied, and such may vary. In some embodiments, the laser system includes a solid-state laser. In other embodiments, the laser system includes a fiber laser. In still other embodiments, the laser system includes a ytterbium-doped fiber laser. The laser system may be configured to generate light energy having any convenient wavelength and/or pulse duration and/or power output, and such may vary. In embodiments, the laser system is configured to generate light energy having a wavelength ranging from 350 nm to 5.0 μm, pulse duration of 1 to 1,000 femtoseconds, maximum power output of greater than 100 Watts, and a pulse repetition ranging between 1 Hz to 1,000 MHz. In still other embodiments, the laser system includes dispersion compensation. By dispersion compensation, it is meant, applying any convenient technique, such as applying optical elements, to control or cancel or compensate the chromatic dispersion of the light energy emitted by the laser system and/or generated by the optical system and tissue downstream of the laser system. Dispersion compensation may be achieved by, for example, manually applying optical components internal or external to the laser.

In embodiments, the excitation source comprises a second laser. Any convenient laser, capable of transmitting sufficient light energy at the appropriate wavelength through non-transparent tissue, such as ocular or periocular tissue, may be applied, and such may vary. Such a second laser may emit light energy in conjunction with or separate from or otherwise complementary to light energy emitted from a first laser, such as first lasers described above. In some cases, the second laser is a titanium-doped sapphire laser. In other cases, the second laser is a fixed wavelength laser. For example, the second laser is a fixed 1,045 nm laser. In embodiments, the second laser is configured to generate light energy having a specified power output. For example, the second laser may be configured to generate light energy having a maximum power output exceeding 100 W. In other embodiments, the second laser comprises a specified usable power band. For example, the second laser may comprise a usable power band of 350 nm to 5.0 μm. In certain cases, the second laser may be configured for imaging and/or for laser treatment. In other cases, the second laser could be an OPA (optical parametric amplifier) configured to extend the wavelength range of the first laser.

In embodiments, the excitation source comprises a third component. Any convenient third component, capable of transmitting sufficient light energy through non-transparent tissue, such as ocular or periocular tissue, may be applied, and such may vary. Such a third component may emit light energy in conjunction with or separate from or otherwise complementary to light energy emitted from one or both of a first laser and a second laser, such as the first and second lasers described above. In some cases, the third component comprises a fixed wavelength excitation source. For example, the third component may comprise a 1.7 μm excitation source. In other cases, the third component comprises Raman shifting of 1.5 μm light in a large mode area photonic crystal rod. In still other cases, the third component emits excitation wavelengths of a fixed wavelength or range thereof. For example, the third component may emit excitation wavelengths between 350 nm and 5 μm. Other known or yet to be discovered or implemented excitation sources or lasers or laser systems or optical systems may be applied as desired for the excitation source of embodiments of the present invention, such as the first, second or third lasers described above.

Excitations sources of interest include a two-part laser system enabling imaging tissue (as well as manipulating tissue, such as providing laser treatment of tissue, as described in detail herein) through non-transparent tissue, such as ocular or periocular tissue. Such embodiments may comprise an optical parametric amplifier (OPA) used to extend the tuning range of a Ytterbium amplified laser. By optical parametric amplifier (OPA), it is meant a laser light source, such as commercially available laser technologies, including common, commercially available light sources, including those used for photo disruption or micromachining, that emits light of variable wavelengths by any convenient optical parametric amplification process. In some cases, a tuning range of an excitation source comprising such a two-part laser system includes 350 nm to 5 μm. However, tuning ranges may vary depending on specific characteristics of the underlying technology and is expected to change as the underlying technology changes and evolves.

In some embodiments, an excitation source, such as a two-part laser system (i.e., first and second lasers) described above, may comprise a wavelength tuning range of 350 nm to 5 μm. With respect to such a tuning range corresponding to certain embodiments, a useful window, or bandwidth, for imaging through non-transparent ocular or periocular tissue includes approximately 1,300 to 1,700 nm. The other frequency windows, or bandwidths, within such a tuning range may be applicable in connection with manipulating tissue, such as providing a laser treatment of tissue, as described in detail below, and, in particular, applicable in connection with thermal energy deposition.

In embodiments, the excitation source may comprise one or more next-generation laser sources. In some cases, the excitation source comprises a conventional titanium-doped sapphire laser, such as SpectraPhysics MaiTai HP DeepSee or similar offerings, with usable power band ranging from 690 to 970 nm. In other cases, the excitation source comprises an extended-infrared ytterbium-doped fiber laser, such as SpectraPhysics Insight X3 or similar offerings, with usable power band ranging from 690 to 1300 nm. In still other cases, the excitation source comprises an optical parametric amplified ytterbium laser, such as Coherent Monaco-Opera F or similar offerings, with usable power band ranging from 600 to 2,500 nm.

In some embodiments, deploying the excitation source comprises steering the focus of the excitation source over a predetermined area, i.e., an area of interest. For example, the excitation source may be moved over a specified area in order to transmit light energy to the structure corresponding to the specified area over which the excitation source is moved such that the structure can be imaged over such corresponding specified area. Similarly, in other embodiments, deploying the excitation source comprises articulating the excitation source around non-transparent tissue, such as ocular or periocular tissue. That is, the excitation source may be articulated around the non-transparent tissue, such as ocular or periocular tissue, in order to transmit light energy to the structure corresponding to area over which the excitation source is articulated such that the structure can be imaged over such corresponding area. By articulating the excitation source, it is meant moving the excitation source in any desired manner, such as any combination of translating and/or rotating the excitation source in three dimensions.

In embodiments, excitation sources of interest comprise one or more pulsed lasers. By pulsed laser, it is meant any convenient laser that is not a continuous laser. For example, a pulsed laser may be configured so that the optical power appears in pulses of some duration at some repetition rate or at a specified duty cycle. In some embodiments, the pulsed laser may generate light energy having a pulse duration lasting from 1 to 1,000 femtoseconds. In other embodiments, the pulsed laser may generate light energy having a pulse duration lasting less than 300 femtoseconds, such as between 30 to 300 femtoseconds. In some embodiments, pulse duration lasting between 40 to 150 femtoseconds may be applied in connection with imaging structures through non-transparent tissue, such as ocular or periocular tissue. In embodiments, in order to provide sufficient energy for imaging structures through non-transparent tissue, such as ocular or periocular tissue, the pulse repetition rate of a pulsed laser is approximate 1 MHz. However, any convenient pulse repetition rate may be applied, and such may vary, for example, based on the underlying technology or based on the underlying non-transparent tissue, such as ocular or periocular tissue, i.e., to provide sufficient pulse energy therefor. In other embodiments, the pulse repetition rate of a pulsed laser may range from 1 Hz to 1,000 MHz, such as 1 Hz to 65 MHz. Certain embodiments may transmit light energy with a per pulse energy of up to approximately 700 μJ, such as, for example, a pulsed laser with an approximately 600 μJ pulse energy corresponding to low pulse repetition rates of less than 5 kHz, such as 2 kHz. Other embodiments may transmit light energy with average power output greater than 100 Watts.

In embodiments, the excitation source comprises additional optical components. Such additional optical components may facilitate transmitting light energy to a structure through non-transparent tissue, such as ocular or periocular tissue, for imaging such structure. Exemplary optical components include, but are not limited to, a laser scanner to move the laser focus throughout the image field in any desired scan pattern or raster. Other exemplary optical components include, but are not limited to, piezo electric components to rapidly control a focus position along an optical axis, a pulse compressor for shortening a pulse width, a power attenuator or adaptive optics for wavefront shaping, as described in detail herein. Exemplary optical components include those used in connection with LASIK and femto-mediated cataract surgery for steering lasers rapidly to different positions, as such optical components are known in the art. In embodiments, such optical components may be packaged with a laser of the excitation source of the embodiment but may not be fundamental to the laser technology itself.

Upon completion of deploying the excitation source in step 104, the process moves to step 106 next.

### Emitted Light:

At step 106, light emitted from the structure is detected. That is, the effect of the light energy transmitted to the structure through non-transparent tissue, such as ocular or periocular tissue, at step 104, is that light is emitted from the structure via multiphoton excitation. In embodiments, light emitted from the structure via multiphoton excitation is itself transmitted through the non-transparent tissue, such as ocular or periocular tissue, as described above, prior to detection.

In embodiments, the light emitted from the structure via multiphoton excitation comprises stimulated light. By stimulated light, it is meant that, in embodiments, light transmitted from the excitation source interacts with a particle at a molecular or subatomic level, liberating energy and thereby creating a photon. In some embodiments, stimulated light is excited via a higher-order nonlinear process. In some cases, the higher-order nonlinear process comprises one or more of: 2-photon excited fluorescence (2PEF) or second harmonic generation (SHG) or 3-photon excited fluorescence (3PEF) or third harmonic generation (THG) or excitation of a molecule to emit/stimulate light generation via greater than three photons (e.g., four-photon excitation (4PEF) or fourth harmonic generation (4HG)).

In certain embodiments, light emitted via multiphoton excitation comprises light emitted from endogenous fluorophores present in the imaged structure. In other embodiments, light emitted via multiphoton excitation comprises light emitted from exogenous fluorophores present in the imaged structure. In such embodiments, any convenient endogenous fluorophores or exogenous fluorophores may be leveraged or introduced into the structure, as the case may be. In some cases, the fluorophores emit one or more of ultra-violet, blue, green, red or far-red light.

With respect to multiphoton excitation, in some embodiments, the light emitted from the structure comprises light emitted via two-photon excitation (2PEF) and further comprises a second harmonic generation (SHG) signal. In other embodiments, the light emitted from the structure comprises light emitted via three-photon excitation (3PEF) and further comprises a third harmonic generation (THG) signal.

In some embodiments, longer laser wavelengths of the excitation source enable penetration of excitation light through non-transparent tissue, such as, e.g., the sclera. Depending on the fluorophore applied in an embodiment, the same excitation wavelength may be used to excite the fluorophore with two photons, three photons, or more than three photons.

In embodiments, the same lens, such as an objective lens, may be used to: (i) transmit light energy from the excitation source to a structure through non-transparent tissue, such as ocular or periocular tissue, as well as (ii) transmit light emitted from the structure via multiphoton excitation through the non-transparent tissue, such as ocular or periocular tissue, i.e., to one or more sensors or detectors. In other embodiments, the same objective lens need not be used for both such light paths. In some cases, signal is collected (for detection via a detector) at a location that differs from that location where light emitted by the excitation source is transmitted into the non-transparent tissue, such as ocular or periocular tissue. In some cases, signal is collected (for detection via a detector) at any convenient location and such may vary. For example, signal may be collected by placing a sensor over the cornea. In embodiments, such a sensor collects signal light that is emitted deep within the tissue, such as ocular or periocular tissue, by means other than signal light that is collected by an objective lens used to transmit light emitted by the excitation source to the non-transparent tissue, such as ocular or periocular tissue. By light emitted deep within the tissue, such as ocular or periocular tissue, it is meant signal light generated by multiphoton excitation which is emitted at the laser focus. In some cases, signal light may be collected from multiple detectors at any convenient location. For example, some embodiments comprise both a sensor over the cornea as well as signal light collected through the same objective lens used to transmit light from the excitation source into the tissue. In general, in embodiments, an image of a structure is formed by moving the focus of light emitted by the excitation source through the tissue, such as ocular or periocular tissue, over a three-dimensional volume. The signal generated at the focus of the excitation source may be collected by sensors. Such sensors may be positioned anywhere, and, in general, the more emitted light that is collected, the better the resulting image quality. As the focus of the light emitted from the excitation source is moved over a three-dimensional volume (e.g., by scan mirrors or a translation device or other convenient mechanism), the detected light signal is correlated to the position of the focus of light emitted by the excitation source. Such process may be used to digitally assemble an image voxel by voxel. Unlike, for example, a traditional camera, such image formation does not depend on a specific way of, or location of, collecting signal light.

In some cases, adaptive optics techniques are applied to light energy emitted from the excitation source and/or light stimulated in the imaged structure. Any convenient adaptive optics techniques may be applied. Adaptive optics techniques of interest are described in detail herein.

Upon completion of detecting light emitted from the structure in step 106, the process moves to step 108 next.

### Imaging/Detection:

At step 108, the structure is imaged based on the light detected at step 106. That is, the light emitted from the structure, having been detected, is used (i.e., gathered, combined, associated over a specified area, volume or time period) to generate an image of the structure. In embodiments, the imaging and/or detection steps may be computer-implemented; i.e., a computer processing device may be programed or otherwise employed to combine or order or otherwise arrange light detected from the structure at step 106 in order to generate one or more images of the structure. Any convenient technique or algorithm may be employed to resolve the light emitted from the structure and detected at step 106 into an image of the structure at step 108.

That is, in embodiments, images may be formed by moving the focus of the light transmitted from the excitation source (e.g., a focal point of a laser) through tissue, such as ocular or periocular tissue, in three-dimensional space, i.e., over a volume that includes the structure to be imaged. The signal generated at the focus of the light transmitted by the excitation source is collected by sensors, i.e., detectors, as described herein, and the sensors could be positioned in any convenient location capable of receiving stimulated light emitted by the imaged structure. Such a detector may comprise a contact lens, a filter and a photomultiplier tube (PMT) or the like positioned, e.g., directly on the cornea. In embodiments, traditional detection arrangements may be applied, in which light is collected through the same objective lens used to deliver excitation light. As the focus of the excitation source is moved in three-dimensional space, e.g., by translation or articulation of an optical system relative to the tissue, such as ocular or periocular tissue, and/or by the scan mirrors, the detected signal may be correlated with the position of the focus of the excitation source, which is used to digitally assemble the image pixel-by-pixel. For visualization purposes, imaging the structure by such pixel-by-pixel assembly in embodiments is analogous to applying a very high-resolution dot-matrix printer in three dimensions.

In embodiments, the imaging comprises subcellular resolution.

### Spatial Imaging:

Certain embodiments of the present invention comprise imaging the structure over a specified volume. Any convenient volume may be selected, and such may vary, depending, for example, on the volume of the structure or phenomenon to be imaged. In some cases, imaging the structure over a specified volume comprises gathering imaging data at a specified spatial resolution. Any convenient specified spatial resolution may be applied, and such may vary, depending, for example, on feature sizes of the structure it is desired to image.

In some cases, deploying an excitation source to transmit light energy to a structure comprises spatially guiding the excitation source to transmit light energy through non-transparent tissue, such as ocular or periocular tissue. In some cases, spatially guiding the excitation source to transmit light energy to a structure comprises using a multi-modal laser scanner to guide the excitation source. That is, the laser scanner may be used to steer the focus of the excitation laser to different locations. Since, in embodiments, the relative position of the laser focus is known, the image is computed by moving the laser focus across the desired field, thereby, essentially generating an image pixel by pixel by moving the focus back and forth (i.e., “laser scanning”). Any convenient multi-modal laser scanner or technique for laser scanning may be employed, and such may vary. In some cases, the multi-modal laser scanner is switchable between resonant imaging and patterned point-scanning.

Embodiments of the present invention may apply laser scanning techniques, such as patterned laser scanning in conjunction with computational methods for combining the results of laser scanning. Some embodiments further comprise employing line-scanning particle image velocimetry (LS-PIV). In such embodiments, employing line-scanning particle image velocimetry (LS-PIV) may comprise measuring the flow of fluid through certain tissue or structure, such as, e.g., measuring blood flow. In such cases, measuring blood flow may comprise measuring choroid blood flow. In general, patterned laser scanning techniques enable organized and/or systemic laser scanning of the excitation laser. Such techniques allow for measurement of a biological phenomenon, such as, for example, the velocity of blood cells within vessels. A next step in applying such patterned scanning techniques may comprise applying computational methods on the unique image data sets obtained via patterned scanning. Embodiments employing such laser scanning and computational analysis techniques may be referred to as applying Particle Image Velocimetry.

In connection with imaging the structure over a specified volume, an embodiment of the present invention is a method of imaging nerve structures. That is, embodiments of the present invention may relate to visualizing nerve structures, in some cases, in connection with pain management, i.e., directing treatment or directing a procedure with the benefit of visualizing nerve structures or pain receptors.

Embodiments of the systems, devices, and methods disclosed herein are configured to obtain and to process, i.e., analyze, images such that clinically relevant imaging latencies can be obtained. That is, spatial imaging can be obtained using embodiments of the presently claimed invention with clinically relevant processing times. In some cases, real time or near real time spatial imaging can be obtained using embodiments of the present invention.

### Temporal Imaging:

Certain embodiments of the present invention comprise imaging the structure over a specified period of time. Any convenient time period may be selected, and such may vary, depending, for example, on the time scale of the phenomenon it is desired to image. In some cases, imaging the structure over a specified period of time comprises gathering imaging data at a specified temporal resolution or a specified pulse repetition rate. In embodiments, pulse repetition rate is relevant for speed of imaging or laser treatment. Any convenient temporal resolution or pulse repetition rate may be applied, and such may vary, depending, for example, on the temporal resolution of the phenomenon to be imaged. In certain cases, the pulse repetition rate is between 1 Hz to 1,000 MHz, such as between 1 kHz to 1,000 kHz.

In connection with imaging the structure over a specified period of time, an embodiment of the present invention is a method of imaging cellular dynamics. In some cases, an embodiment of the present invention is a method of imaging neuronal activity, such as neuronal activity with calcium indicators. In still other cases, an embodiment of the present invention is a method of imaging hemodynamics. In yet other cases, an embodiment of the present invention is a method of imaging blood flow within micro-vasculature.

Embodiments of the systems, devices, and methods disclosed herein are configured to obtain and to process, i.e., analyze, images such that clinically relevant imaging latencies can be obtained. That is, temporal imaging can be obtained using embodiments of the presently claimed invention with clinically relevant processing times. In some cases, real time or near real time temporal imaging can be obtained using embodiments of the present invention.

### CBC—Complete Blood Component Analyses Morphological Features, Cell Size, Histological Staining etc.:

Embodiments of the systems, devices, and methods disclosed herein, including embodiments related to blood flow or flow analysis, are capable of detecting blood cells or blood cell types and related statistics. Blood cells and blood cell types that may be detected by the systems, devices and methods disclosed herein include, without limitation, red blood cells, hemoglobin, white blood cells (including neutrophils, lymphocytes, monocytes, eosinophils, and basophils), platelets, reticulocytes, and nucleated red blood cells. Various measurements of different blood components may be performed, including, but not limited to, cell count, cell size, cell complexity, granularity, hematocrit, mean corpuscular volume, mean corpuscular hemoglobin, and mean corpuscular hemoglobin concentration. In some embodiments, the above disclosed measurements may be performed using stain independent methods in the absence of histological staining.

Embodiments of the systems, devices and methods include extracting stain-independent features from histologically stained specimens to determine complete blood component analysis. Histologically stained specimens include those biological samples and/or bodily fluids prepared with histological stains for analysis of cell morphology. In some instances, histologically stained specimens include but are not limited to hematological samples prepared from blood and containing cell types or elements that could be found in blood including but not limited to nucleated blood cells, enucleated blood cells, white blood cells, nucleated red blood cells, red blood cells, giant platelets, leukocytes, basophils, eosinophils, lymphocytes, monocytes, neutrophils, platelets, mature or immature blood cells, malignant or tumor cells, parasites, bacteria, etc.

In some instances, methods described herein may include preparing a histologically stained specimen from a subject where the specimen contains histologically stain cells or is prepared to include histologically stain cells. In other instances, the specimen may be previously prepared and the method may include processing a digital image obtained from a histologically stained specimen from a subject.

As used herein, histology stains refer to those stains used in microscopic analysis of the cellular anatomy and/or morphology of cells obtained from a multicellular organism. Histology stains generally include at least one dye that stains one or more cell types and/or components of one or more cell types a contrasting color. Histology stains may also include at least one counter-stain that stains the rest of the cells or the rest of the cell a different color. Histological techniques, stains and staining methods are well-known and include but are not limited to those described in Kierman. Histological and histochemical methods: Theory and practice. Oxford: Butterworth/Heinemann, 1999 and Bancroft & Stevens. Theory and practice of histological techniques. New York, N.Y.: Churchill Livingstone, 1996; the disclosures of which are incorporated herein by reference in their entirety.

Histological staining techniques can be specific, staining one or more particular cells in a specific way, or non-specific, staining essentially all cells or most cells in the same or similar way. Histology stains include but are not limited to e.g., Alcian blue stains, Aniline blue stains, Azan stains, Biebrich scarlet-acid fuchsin stains, Carbol-fuchsin stains, Chrome alum/haemotoxylin stains, Congo Red stains, Crystal violet stains, Fast Red stains, Hematoxylin and Eosin (H&E) stains, Iron Hematoxylin stains, Isamin blue/eosin stains, Jenner's stains, Mallory's Phosphotungstic Acid Hematoxylin (PTAH) stains, Mallory's Trichrome stains, Masson stains, Malachite Green stains, Methyl Green-Pyronin (MGP) stains, Nissl and methylene blue stains, Nissl stains, Oil Red O stains, Orcein stains, Osmic Acid stains, Osmium Tetroxide stains, Papanicolaou stains, Periodic Acid-Schiff (PAS) stains, Reticulin stains, Romanowsky stains, Safranin O stains, Silver stains, Sudan Black and osmium stains, Toluidine-blue stains, Trichrome AB, Trichrome LG, Trypan Blue stains, van Gieson stains, Verhoff's stains, Weigert's resorcin-fuchsin stains, and the like.

Dyes included in histology stains will vary depending on the stain formulation and the desired staining result. In some instances, dyes useful in histology stains may include but are not limited to, e.g., Acid Fuchsin calcium salt, Acid fuschin, Alcian Blue, Alizarin Red, Aniline blue, Aniline Blue diammonium salt, Auramine O Dye, Azure, Azure A chloride, Azure B, Basic Fuchsin, Bismarck Brown Y, Brilliant Cresyl Blue, Brilliant Green, Carmine, Congo Red, Cresyl Violet acetate, Crystal Violet, Darrow Red, Eosin, Eosin B, Eosin Y, Eosin Y disodium salt, Erythrosin B, Erythrosin extra bluish, Ethyl eosin, Fast Green FCF, Hematoxylin, Indigo carmine, Janus Green B, Light Green SF Yellowish, Malachite Green oxalate salt, Methyl Blue, Methyl green, Methyl Green zinc chloride, Methyl Orange, Methyl violet 2B, Methylene blue, Methylene Violet (Bernthsen), Neutral Red, Nigrosin, Nile Blue A, Oil Red O, Orange G, Orange Il sodium salt, Orcein synthetic, Phloxine B Dye, Pyronin B, pyronin G, Pyronin Y, Resazurin sodium salt, Rose Bengal sodium salt, Safranin O, Sudan Black B, Sudan III, Sudan IV, Thionin acetate salt, toluidine, Toluidine Blue O, and the like.

Histological stains include Romanowsky stains. Romanowsky stains are generally neutral stains composed of various components including but not limited to methylene blue (e.g., Azure B) and eosin (e.g., Eosin Y) dyes. Azures are basic dyes that bind acid nuclei and result in a blue to purple color. Eosin is an acid dye that is attracted to the alkaline cytoplasm producing red coloration. Romanowsky stains vary and include various formulations including those containing various azure and eosin analogs. Romanowsky stains and their mechanisms of staining are well-known and described in e.g., Horobin & Walter. Histochemistry (1987) 86:331-336; Marshall et al. J Clin Pathol (1978) 31(3): 280-2; Marshall et al. J Clin Pathol. (1975) 28(11): 920-3; J Clin Pathol (1975) 28(8): 680-5; the disclosures of which are incorporated herein by reference.

Romanowsky stains include but are not limited to Giemsa Stain, Wright Stain, Wright Giemsa Stain, Jenner Stain, Jenner-Giemsa Stain, Leishman Stain, May Grunwald Stain, May Grunwals Giemsa Stain, and the like. Each Romanowsky stain may exist in various formulations either as derived from various different recipes or as supplied from various providers. Romanowsky stain formulations may include various stain components including but not limited to e.g., methylene blue, azure A, azure B, azure C, toluidine blue, thionine, methylene violet Bernthsen, methyl thionoline, thionoline, eosin, eosin Y, tribromofluorescein, fluorescein, thiazine dyes, and the like. Romanowsky stain formulations may include various solvents to dissolve stain components including aqueous and organic solvents including but not limited to e.g., water and alcohols including but not limited to e.g., methanol, ethanol, isopropyl alcohol, etc. The histological stains and components thereof include those commercially available from such suppliers including not limited to e.g., Sigma Aldrich, Thermo Fisher Scientific, Avantor Performance Materials, VWR International, Polysciences Inc., and the like.

Subjects from which a specimen may be acquired include but are not limited to human subjects, mammalian subjects (e.g., primates (apes, gorillas, simians, baboons, orangutans, etc.), ungulates (e.g., equines, bovines, camelids, swine, etc.), canines, felines, rodents (mice, rats, etc.), etc. Specimens may include biological fluid samples and biological samples which may be processed prior to imaging, e.g., processed onto a slide and histologically stained. In instances where the specimen is a blood sample the sample may be processed into a blood smear and stained with a hematological stain.

Embodiments of the systems, devices, and methods disclosed herein, including embodiments related to blood flow or flow analysis, are capable of detecting structures within tissues not limited to blood, as described above. Embodiments may be configured to detect changes with respect to underlying tissues and may utilize such capacity to detect changes in tissue to identify underlying structures of tissue or the identify or distinguish underlying disease conditions, e.g., cancerous cells. Embodiments may be configured to utilize any negative contrast image, such as negative contrast images gleaned from multiphoton imaging techniques of the present disclosure, to identify substructures within such negative contrast images.

Upon completion of imaging the structure based on the detected light in step 108, the process ends at step 110.

FIG. 1B illustrates a flow diagram 120 for imaging a structure through non-transparent tissue, such as ocular or periocular tissue, according to another embodiment of the present invention. The embodiment of the present invention depicted in FIG. 1B relates to imaging any convenient structure accessible through non-transparent tissue, such as ocular or periocular tissue, according to the present techniques and such may vary. Flow diagram 120 is an exemplary embodiment of the present invention provided for illustrative purposes, and the structure as well as the optical technique applied may vary as desired in embodiments of the present invention. Certain steps depicted in flow diagram 120 are similar or identical to those illustrated in connection with the embodiment depicted by flow diagram 100 in FIG. 1A. Descriptions of such similar or identical steps will not be duplicated in connection with the discussion of FIG. 1B. Further, flow diagram 120 presents certain additional steps present in embodiments of the invention. However, in some embodiments of the present invention, certain of the steps presented in flow diagram 120 need not be implemented, as will be apparent to those skilled in the art.

Flow diagram 120 starts at step 122. From starting step 122, the process proceeds next to step 124.

### Fluorophores/Fluorescent Dye:

At step 124, fluorophores are introduced into the structure to be imaged. As described herein, in embodiments, introducing fluorophores into the structure comprises introducing fluorescent dye into the imaged structure or labeling blood plasma present in the imaged structure with a fluorescent dye. Any convenient fluorescent dye may be applied, such as a dye comprising particles, such as fluorophores, that emits stimulated light in response to receiving light transmitted from the excitation source. Any convenient commercially available fluorophores may be applied, and such may vary. In general, introducing exogenous fluorophores into the structure may be a technique used to increase light emitted from the structure via multiphoton excitation such that such emitted or stimulated light is capable of being transmitted through non-transparent tissue, such as ocular or periocular tissue, such that it is detectable for purposes of imaging the structure. In other cases, endogenous fluorophores may be leveraged to a similar effect.

Upon completion of introducing fluorophores into the structure in step 108, the process moves next to step 126.

At step 126, an excitation source is deployed. Step 126 is identical to step 104 described above in connection with flow diagram 100 of FIG. 1A.

Upon completion of deploying an excitation source in step 126, the process moves next to step 128.

At step 128, light emitted from the structure is detected. More specifically, in the embodiment, light emitted from at least the fluorophores introduced at step 124 and present in the structure is detected. Such emitted or stimulated light may be detected in any convenient manner, such as those techniques and aspects described above in connection with step 106 of flow diagram 100 in FIG. 1A.

In embodiments, instead of introducing fluorophores at step 124, endogenous fluorophores may instead be used in connection with imaging the structure. By endogenous fluorophore, it is meant any component (e.g., a molecule, a protein a pigment or the like) capable autofluorescence when exposed to an excitation source of the present invention. In embodiments, endogenous fluorophores may be present in the imaged structure and the excitation source may be configured to stimulate autofluorescence from such endogenous fluorophores in order to image the structure. That is, in embodiments, step 124 may comprise identifying an endogenous fluorophore present in the imaged structure and/or configuring the excitation source to emit light with specific characteristics that allow the endogenous fluorophore to initiate autofluorescence. Endogenous fluorophores of interest include, but are not limited to flavins, i.e., derivatives of riboflavin, such as flavin mononucleotide (FMN), flavin adenine dinucleotide (FAD), intracellular riboflavin, flavin coenzymes and flavoproteins. Other endogenous fluorophores of interest include, but are not limited to nicotinamide-adenine dinucleotide (NADH) and nicotinamide-adenine dinucleotide phosphate (NADPH), lipofuscin, elastin and collagen. Background information, including regarding endogenous fluorophores, is presented in: Billinton N, Knight A W. Seeing the wood through the trees: a review of techniques for distinguishing green fluorescent protein from endogenous autofluorescence. Anal Biochem. 2001 Apr. 15; 291(2): 175-97. doi: 10.1006/abio.2000.5006. PMID: 11401292; and Aubin J E. Autofluorescence of viable cultured mammalian cells. Journal of Histochemistry & Cytochemistry. 1979; 27(1): 36-43. doi: 10.1177/27.1.220325, the disclosures of each of which are incorporated herein in their entireties. Background information, including regarding leveraging endogenous contrast in the context of second harmonic generation, is presented in: Chen, X., Nadiarynkh, O., Plotnikov, S. et al. Second harmonic generation microscopy for quantitative analysis of collagen fibrillar structure. Nat Protoc 7, 654-669 (2012). https://doi.org/10.1038/nprot.2012.009; Lim H. Harmonic Generation Microscopy 2.0: New Tricks Empowering Intravital Imaging for Neuroscience. Front Mol Biosci. 2019 Oct. 9; 6:99. doi: 10.3389/fmolb.2019.00099. PMID: 31649934; PMCID: PMC6794408; and Campagnola P J, Loew L M. Second-harmonic imaging microscopy for visualizing biomolecular arrays in cells, tissues and organisms. Nat Biotechnol. 2003 Nov; 21(11): 1356-60. Doi: 10.1038/nbt894. PMID: 14595363, the disclosures of each of which are incorporated herein in their entireties. Background information, including regarding leveraging endogenous contrast in the context of third harmonic generation, is presented in: Rehberg M, Krombach F, Pohl U, Dietzel S (2011) Label-Free 3D Visualization of Cellular and Tissue Structures in Intact Muscle with Second and Third Harmonic Generation Microscopy. PLoS ONE 6(11): e28237. https://doi.org/10.1371/journal.pone.0028237; H. Lim, et al. Label-free imaging of Schwann cell myelination by third harmonic generation microscopy. PNAS Dec. 1, 2014 111(50 ) 18025-18030 https://doi.org/10.1073/pnas.1417820111; and Yildirim, M., Sugihara, H., So, P. T. C. et al. Functional imaging of visual cortical layers and subplate in awake mice with optimized three-photon microscopy. Nat Commun 10, 177 (2019). https://doi.org/10.1038/s41467-018-08179-6, the disclosures of each of which are incorporated herein in their entireties.

Embodiments of the present invention may utilize second harmonic generation (SHG), third harmonic generation (THG) or higher order processes, such as, for example, fourth harmonic generation or harmonic generation greater than fourth harmonic generation. In general, harmonic generation refers to nonlinear optical processes in which: (i) a number of photons with the same frequency interact with a nonlinear material; (ii) such photons are “combined;” and (iii) as a result of such combination, such photons generate a new photon having a multiple of the energy of the initial photons, where such multiple corresponds to the number of interacting photons. For example, second harmonic generation is a nonlinear optical process in which two photons with the same frequency interact with a nonlinear material, are “combined,” and generate a new photon with twice the energy of the initial photons that conserves the coherence of the excitation.

Upon completion of detecting light emitted from the structure in step 128, the process moves next to step 130.

### Adaptive Optics:

At step 130, an adaptive optics technique is employed, in connection with imaging the structure, to the light used for multiphoton excitation of the structure detected at step 128. Any convenient adaptive optics technique may be employed, and such may vary. For example, adaptive optics techniques may be employed to reduce the effect of incoming wavefront distortions associated with, for example, multiphoton excitation by the structure.

In embodiments, adaptive optics are used to shape the wavefront of the excitation light, not the detected light. This allows maximum efficiency of excitation at the smallest possible focus, which in turn allows improved efficiency of signal generation at the smallest point, which also maximizes resolution. While adaptive optics techniques may be employed in connection with signal collection, in embodiments, for image signal collection, adaptive optics need not be employed and in some cases distortions do not hinder image collection since embodiments only need to collect as much signal as possible. In embodiments, image formation is extrapolated from the known position of laser focus. As such, in certain cases, step 130 may be initiated as part of, or immediately after, step 126, in which the excitation source is deployed to emit excitation energy, and in such cases, step 130 may be performed prior to step 128, in which signal light is detected from the structure.

In embodiments, adaptive optics technique may be employed that increase efficiency of multiphoton excitation. By increasing efficiency of multiphoton excitation, it is meant increasing the efficiency of generating light emitted by the structure via multiphoton excitation. For example, in embodiments, adaptive optics (AO) through direct sensing and correction of wavefront distortions can be employed to further enhance the resolution of multiphoton microscopies deep into non-transparent and scattering tissues to achieve sub-cellular and diffraction-limited resolution. Further details are provided in: Wang, K., et al., Direct wavefront sensing for high-resolution in vivo imaging in scattering tissue. Nat Commun, 2015. 6: p. 7276, the disclosure of which is incorporated herein in its entirety. In some cases, the adaptive optics technique is further configured to improve a resolution of the imaged structure. In embodiments, the adaptive optics technique comprises one or more of: direct sensing or direct wavefront sensing or correction of wavefront distortions or an image point-spread function or a laser guide star technique or an indirect technique. In such embodiments, direct wavefront sensing may comprise employing a Shack-Hartman sensor and a deformable mirror. Direct wavefront sensing can be combined with multiphoton imaging to improve morphological and functional imaging deeper into highly scattering tissues. In embodiments, direct-wavefront-sensing is performed with a Shack-Hartman sensor to measure the wavefront distortion of a fluorescent guide star created inside the specimen. This information can be relayed to a deformable mirror to reshape the excitation wavefront and compensate for the aberrations to achieve the tightest excitation focus possible. In embodiments, indirect-wavefront-sensing is performed without the use of point guide stars or Shack-Hartman sensor, through a feedback loop which modulates the excitation wavefront to maximize emission power. Further background information is provided in: Jung, S., et al., Analysis of fractalkine receptor CX(3)CR1 function by targeted deletion and green fluorescent protein reporter gene insertion. Mol Cell Biol, 2000. 20(11): p. 4106-14, the disclosure of which is incorporated herein in its entirety.

In some cases, adaptive optics techniques of interest comprise indirect (e.g., algorithmic) techniques for determining the optimal excitation wavefront. Further details are provided in: Debarre D, Botcherby E J, Watanabe T, Srinivas S, Booth M J, Wilson T. Image-based adaptive optics for two-photon microscopy. Opt Lett. 2009 Aug. 15; 34(16): 2495-7. doi: 10.1364/ol.34.002495. PMID: 19684827; PMCID: PMC3320043; and Ji N, Milkie D E, Betzig E. Adaptive optics via pupil segmentation for high-resolution imaging in biological tissues. Nat Methods. 2010 Feb; 7(2): 141-7. doi: 10.1038/nmeth.1411. Epub 2009 Dec. 27. PMID: 20037592; and Tang J, Germain R N, Cui M. Superpenetration optical microscopy by iterative multiphoton adaptive compensation technique. Proc Natl Acad Sci U S A. 2012 May 29; 109(22): 8434-9. doi: 10.1073/pnas.1119590109. Epub 2012 May 14. PMID: 22586078; PMCID: PMC3365222, the disclosures of each of which are incorporated herein by reference.

In other embodiments, the adaptive optics technique is configured to improve spatiotemporal resolution. With respect to a guide star technique, in embodiments, the guide star is representative of an image point-spread function and is scanned along all the positions in the image (i.e., according to a raster). The distortion/blurring of the guide star is used to extrapolate how the wavefront should be corrected through adaptive optics (AO) approaches to determine and achieve the best resolution.

With respect to an indirect approach, the excitation wavefront can be modified via optimization of an objective function, the most successful of which segments the image at the objective plane into independent subregions on account of the spatially localized nature of inhomogeneities, to maximize either the emission intensity, focal radius or spatial frequency. Further details regarding an indirect adaptive optical approach which determines the optimal excitation wavefront based on focal radius (less blur is better) are found in Booth, M. J.

Wavefront sensorless adaptive optics for large aberrations. Opt. Lett. 32, 5-7 (2007), the disclosure of which is incorporated herein. Further details regarding an indirect adaptive optical approach in which the objective function says: “Living things are being viewed here, not TV static. The less high frequency garbage present in the image, the better,” are found in Debarre, D., Booth, M. J. & Wilson, T. Image based adaptive optics through optimisation of low spatial frequencies. Opt. Express 15, 8176-8190 (2007), the disclosure of which is incorporated herein.

Upon completion of employing an adaptive optics technique in step 130, the process may move next to step 132 or may return to step 126. In the event the application of the adaptive optics technique yields sufficient improvements in the ability to image the structure (e.g., sufficient improvements in image resolution of the structure), then the process moves next to step 132. In the event the application of the adaptive optics technique fails to yield sufficient improvement in the ability to image the structure (e.g., insufficient improvement in image resolution of the structure), then the process may return to step 126, where changes to light emitted by the excitation source or changes to the adaptive optics techniques employed may be made in order to achieve sufficient improvements (e.g., sufficient improvement in resolution of the structure). Such return to step 126 may be repeatedly applied such that iterative changes to, for example, the light emitted by the excitation source or the adaptive optics technique, may be implemented.

### Manipulating Tissue:

At step 132, the structure imaged in connection with one or more of steps 124, 126, 128 and 130 is manipulated. As described above, embodiments of the present invention further comprise manipulating the imaged structure. In embodiments, manipulating the imaged structure comprises using the excitation source, i.e., an excitation source, such as those described above in connection with step 104 of flow diagram 100 of FIG. 1A, to manipulate the imaged structure. In embodiments, the same excitation source used to image the structure may be used to manipulate the structure. That is, in such embodiments, a first excitation source may be used to image the structure, and that same first excitation source may also be used to manipulate the structure. In other embodiments, a separate excitation source may be used to image the structure and to manipulate the structure. That is, in such embodiments, a first excitation source may be used to image the structure, and a second excitation source may be used to manipulate the structure.

In embodiments, manipulating the imaged structure comprises using the excitation source for non-incisional therapy. By non-incisional therapy, it is meant applying a therapy without creating an incision, e.g., via a scalpel. In other embodiments, manipulating the imaged structure comprises using the excitation source to disrupt tissue of the imaged structure. In other embodiments, manipulating the imaged structure comprises using the excitation source for photo-tissue interactions. In such cases, the photo-tissue interactions may comprise laser-tissue interactions. In other cases, the laser-tissue interactions comprise laser-tissue perturbations. In certain embodiments, using the excitation source for photo-tissue interactions comprises configuring the excitation source for multi-photon-mediated damage, such as thermal damage.

In other embodiments, using the excitation source for photo-tissue interactions comprises configuring the excitation source for photo-disruption. In still other embodiments, using the excitation source for photo-tissue interactions comprises configuring the excitation source for blood vessel coagulation. In embodiments, manipulating the imaged structure comprises ablating the imaged structure.

In certain embodiments, manipulating the imaged structure comprises using the excitation source to treat glaucoma. In some cases, using the excitation source to treat glaucoma comprises using the excitation source to reduce aqueous production of ocular tissue. In such embodiments, using the excitation source to reduce aqueous production of ocular tissue may comprise damaging the ciliary body to reduce aqueous production, for example, by thermally damaging the ciliary body or applying a photo disruption mediated process. In other embodiments, using the excitation source to treat glaucoma comprises increasing outflow of aqueous humor. In such embodiments, using the excitation source to increase outflow of aqueous humor may comprise performing laser trabeculotomy. In some cases, using the excitation source to increase outflow of aqueous humor comprises performing laser trabeculoplasty directly through the non-transparent tissue of the eye. In contrast, traditional laser trabeculoplasty, i.e., without the benefit of the present invention, is performed through the cornea exclusively.

In embodiments, manipulating the imaged structure comprises using the excitation source to prevent or treat retinal breaks, such as retinal tears. In such embodiments, preventing or treating retinal breaks, such as retinal tears, may comprise identifying areas of interest and providing photocoagulation therapy.

Embodiments of the present invention are configured to prevent or treat retinal breaks in peripheral regions of the retina. Peripheral regions of the retina are typically the part the retina most vulnerable to breaks but also the least accessible part of the retina, and in some cases are inaccessible using traditional techniques.

In other embodiments, using the excitation source for non-incisional therapy comprises providing photocoagulation and thermal treatment to tumors present in tissue, such as ocular or periocular tissue. For example, using the excitation source for non-incisional therapy comprises providing photocoagulation or thermal treatment to ciliary body tumors. In some cases, using the excitation source for non-incisional therapy comprises providing photocoagulation or thermal treatment to peripheral choroidal tumors. In other examples, using the excitation source for non-incisional therapy comprises providing photocoagulation or thermal treatment or ablation to cancer cells, including cancer cells present within dermatologic tissue, such as melanoma.

In still other embodiments, manipulating the imaged structure comprises using the excitation source to optically cross-link scleral tissue. Such embodiments may comprise methods for prevention of myopia or methods for mediation of myopia or prevention of the progression of myopia. In certain embodiments, manipulating the imaged structure comprises using the excitation source to visualize and perform targeted alteration of extraocular muscle function. In other embodiments, manipulating the imaged structure comprises using the excitation source to visualize and perform targeted thermal or photocoagulation therapy of the orbital fat. In embodiments, manipulating the imaged structure comprises using the excitation source to visualize and perform targeted alteration of palpebral tissues.

In embodiments, using the excitation source to manipulate the imaged structure comprises employing one or more of the adaptive optics techniques described above. That is, the adaptive optics techniques described herein may be applied to light energy emitted by the excitation source used to manipulate tissue. Such adaptive optics techniques may be used to make the multi-photon excitation process used in connection with manipulating tissue more efficient or more accurate or more localized, i.e., applied with finer granularity.

Notwithstanding that FIG. 1B depicts that manipulating a structure at step 132 is performed subsequent to imaging a structure, nonetheless, as described herein, manipulating a structure may be performed without previously imaging such structure. That is, embodiments of the present invention comprise manipulating a structure using techniques described herein alone, i.e., without also imaging such structure.

Upon completion of manipulating the imaged structure in step 132, the process may move next to step 134, where the process ends or may return to step 126. In the event it is desired to further image the structure having been manipulated in step 134, as described above, then the process may return to step 126. As described above, in some cases, upon return to step 126, changes to light emitted by the excitation source or changes to the adaptive optics techniques employed may be made in order to achieve improvements (e.g., sufficient improvement in resolution of the structure). Such return to step 126 may be repeatedly applied such that manipulating the structure, as described in step 132 may be iterative and/or interleaved with imaging the structure, as described in steps 126, 128 and 130.

Upon ultimately completing manipulating the imaged structure at step 132, the process ends at step 134.

FIG. 1C illustrates flow diagram 150 for imaging a structure through non-transparent tissue, such as ocular or periocular tissue, according to another embodiment of the present invention. The embodiment of the present invention depicted in FIG. 1C depicts a method of delivery for gene therapy, i.e., using imaging through non-transparent tissue, such as ocular or periocular tissue, to guide delivery of gene therapy to a structure. That is, flow diagram 150 relates to delivery of an active agent, such as, for example, in connection with implementing a gene therapy or a stem cell therapy or an engineered cell therapy technique on the structure imaged through non-transparent tissue, such as ocular or periocular tissue. Gene therapy or stem cell therapy or engineered cell therapy performed in connection with flow diagram 150 may be applied to any convenient structure, such as an ocular or periocular structure. Flow diagram 150 is an exemplary embodiment of the present invention provided for illustrative purposes, and the structure as well as the optical technique applied as well as the agent introduced in connection with the gene therapy or stem cell therapy or engineered cell therapy technique may vary as desired in embodiments of the present invention. Certain steps depicted in flow diagram 150 are similar or identical to those illustrated in connection with the embodiments depicted by flow diagram 100 in FIG. 1A and flow diagram 120 in FIG. 1B. Descriptions of such similar or identical steps are not duplicated in connection with the discussion of FIG. 1C.

Flow diagram 150 starts at step 152. From starting step 152, the process proceeds next to step 154.

At step 154, an excitation source is deployed. Step 154 is identical to step 104 described above in connection with flow diagram 100 of FIG. 1A.

Upon completion of deploying an excitation source in step 154, the process moves next to step 156.

At step 156, light emitted from the structure is detected. Step 156 is identical to step 106 described above in connection with flow diagram 100 of FIG. 1A. Completion of steps 154 and 156 results in generating an image of the structure of interest. Such imaging is used to guide the delivery of an agent to a desired location in subsequent steps 158 and 160. As a result of the imaging accomplished at steps 158 and 160, the delivery of the agent to the desired location can be achieved with a high degree of precision. While steps 154 and 156 are depicted as discrete steps in FIG. 1C, such imaging of the structure of interest may be initiated in connection with steps 154 and 156 and persist over the course of completing steps 158 and 160, such that the structure of interest is continually imaged while a relevant agent is introduced into the structure and the effect of such agent is evaluated, as described below.

Upon completion of detecting light emitted from the structure in step 156, the process moves next to step 158.

### Gene or Cell or Drug Therapy Applications:

At step 158, an agent is introduced into the structure, i.e., the structure of interest. Any convenient agent may be introduced into the structure, i.e., in connection with effecting gene therapy or stem cell therapy or engineered cell therapy in the structure, and such may vary. That is, the agent introduced into the structure may comprise any convenient agent capable of, for example, causing or facilitating a genetic modification in cells of the structure to produce a desired therapeutic effect or to treat a disease of the imaged structure by repairing or reconstructing defective genetic material. The delivery of the agent into the structure is guided, such that the delivery can be precisely located with a high degree of granularity, based on the image data collected in connection with completing steps 154 and 156, described above. In some cases, the structure is continually imaged over the course of delivering an agent to the structure of interest. For example, such imaging may be used to guide a needle capable of delivering an agent to the structure at a precise location.

In some cases, the agent introduced into the structure comprises cells. In such cases, the cells may be stem cells or engineered cells or combinations thereof. In other cases, the agent comprises an active agent, e.g., a pharmaceutical or drug. In yet other cases, the agent may comprise a virus, such as a recombinant virus or biological nanoparticle or viral vectors. In still other cases, the specified agent comprises a molecule. Molecules of interest include DNA, RNA, oligonucleotides, lipoplexes, dendrimers, and inorganic nanoparticles or molecules involved in employing CRISPR tools for gene editing, as such are known in the art. In some cases, one or more agents may be introduced as needed in order to effect gene therapy in the structure. In certain cases, introducing the agent into the imaged structure comprises introducing the specified agent into one or more of: subretinal space, suprachoroidal space, subchoroidal space or intravitreal space. In certain cases, introducing the agent into the imaged structure comprises introducing the specified agent into one or more of: the ciliary body or the stroma of the sclera.

Embodiments of the present invention enable the location of delivery of such one or more agents to be imaged and carefully selected. For example, embodiments of the present invention may be applied to deliver one or more such agents to precise locations in a subretinal space or suprachoroidal space or subchoroidal space or intravitreal space, for example. Embodiments of the present invention may comprise deploying an injector, such as, for example, a needle injector, to introduce one or more agents for use in connection with gene or cell or drug therapy or any other therapy requiring the introduction of an agent, such as an active agent, into a structure. Embodiments of the present invention may further comprise visualizing an aspect of an injector, such as the tip of a needle injector, in conjunction with imaging the structure and delivering such agent.

While not shown in FIG. 1C, in some embodiments, methods comprise imaging a structure using techniques of the present invention prior to, or substantially simultaneously with, introducing an agent into the structure so that the agent may be introduced into a precise location of the structure.

While not shown in FIG. 1C, in some embodiments, methods comprise applying photo cross-linking to aspects of the imaged structure, such as aspects of the sclera. Such photo cross-linking of the sclera, for example, can be used to prevent progression of myopia. Cross-linking agents used in existing techniques, absent the present invention, are applied to the cornea to penetrate the cornea but may not penetrate into the sclera. In cases where such cross-linking agents do not penetrate the sclera, embodiments of the present technique could be applied to inject the agent directly into the sclera, in order to improve the effectiveness of such photo cross-linking techniques to prevent the progression of myopia. Still other embodiments of the present invention use an intravascular chemical that is photo activated by the excitation source of the present invention, e.g., a laser, capable of penetrating non-transparent tissue, such as ocular or periocular tissue, e.g., to target a structure, such as the sclera, in order to achieve photo cross-linking of the sclera.

Upon completion of introducing a specified agent into the structure in step 158, the process moves next to step 160.

At step 160, one or more effects of introducing the agent into the structure are evaluated. In embodiments, at step 160, continued imaging of the structure, initiated at steps 154 and 156, described above, may be used to assess an effect of the specified agent on the structure. That is, in embodiments, imaging the structure is used to evaluate whether the delivery of the agent is achieving an intended effect. Any convenient aspect of the image of the structure may be observed in order to evaluate or assess the effect of the specified agent on the structure; i.e., to evaluate whether the desired gene therapy technique is working or whether the agent was delivered to the desired location. In other embodiments, techniques other than imaging the structure may be applied to evaluate the effectiveness of the delivery of the agent to the desired structure.

Upon completion of evaluating the effect of the agent on the structure, the process may move next to step 162, where the process ends or may return to step 158. In the event it is desired to introduce an additional agent or different agent, e.g., an agent that effects gene therapy in the imaged structure, or introduce an agent to a different location, e.g., a different location of the imaged structure, then the process may return to step 158. As described above, in some cases, upon return to step 158, additional amounts of the same agent previously introduced into the structure or a different agent may be further introduced into the structure. Continuing through the flow diagram after returning to step 158 from step 160 enables an opportunity to evaluate the introduction of additional or different agent or introducing agent into a different location into the structure such that, for example, a gene therapy or stem cell therapy or engineered cell therapy technique may be applied under nearly continuous observation in order to monitor and evaluate the effects of introducing one or more agents into the structure. That is, such return to step 158 may be repeatedly applied such that evaluating the effect of the agent, as described in connection with step 160, may be interleaved with introducing one or more agents into the structure while continuing to image the structure, as described in connection with steps 154, 156 and 158.

Upon ultimately completing evaluation of the effect of the agent on the structure at step 160, the process ends at step 162.

### Flow Analysis Techniques:

Embodiments of the present invention may further comprise advanced computational techniques capable of increasing the spatiotemporal resolution, execution speed and dimensionality of flow analysis using multiphoton imaging. That is, embodiments of flow analysis techniques are capable of not only increasing the speed of execution but also the dimensionality of input and output data. Certain embodiments are configured to provide such flow analysis in real time or substantially in real time or near real time.

Embodiments of the presently claimed invention comprise implementing a signal analysis algorithm that utilizes, for example, a multi-core processor, to concurrently process a sequence of negative contrast images, where such images may be obtained utilizing multiphoton based imaging according to the presently claimed invention. In embodiments, a signal analysis algorithm (e.g., wherein an embodiment of such an algorithm of the present invention is an RBCPIV algorithm) comprises the following two steps: (1) spatial object localization (e.g., by use of n-point Discrete Fourier Transform, i.e., a Fast-Fourier Transform coupled with curve fitting a probability distribution for subpixel accuracy); and (2) physical displacement of an object over time (velocity). In other embodiments a signal analysis algorithm comprises the following steps: after a sequence of negative contrast images are obtained, (1) for any given pair of contrast images (by contrast images, it is meant, for example, one-or two-dimensional frames or three-dimensional volumes; in embodiments, images are represented as an n-dimensional tensor), (2) the n-point Discrete Fourier Transform of each image is taken; (3) a phase weight is computed; (4) the pair of images is cross correlated in Fourier space; (5) a probability distribution (such as, for example, a Gaussian distribution), is fit by solving a non-linear least squares problem (e.g., via the Levenberg Marquardt Algorithm) to each zero frequency shifted image; (6) each fit is used to compute displacement in physical space; and (7) velocity is calculated from physical displacement using time between images. Further details regarding aspects of signal analysis algorithms of the present invention are provided in Levenberg, Kenneth (1944). “A Method for the Solution of Certain Non-Linear Problems in Least Squares.” Quarterly of Applied Mathematics. 2 (2): 164-168. Doi: 10.1090/qam/10666; and Kim T N, Goodwill P W, Chen Y, Conolly S M, Schaffer C B, Liepmann D, Wang R A. Line-scanning particle image velocimetry: an optical approach for quantifying a wide range of blood flow speeds in live animals. PLoS One. 2012;7(6):e38590. doi: 10.1371/journal.pone.0038590. Epub 2012 Jun. 26. PMID: 22761686; PMCID: PMC3383695, in each case, incorporated herein by reference. Further details regarding cross correlation techniques specifically for particle image velocimetry are found in Keane, R. D., Adrian, R. J. Theory of cross-correlation analysis of PIV images. Applied Scientific Research 49, 191-215 (1992). https://doi.org/10.1007/BF00384623, the disclosure of which is incorporated herein by reference.

FIGS. 1D-E illustrate aspects of a signal-analysis algorithm according to embodiments of the present invention. FIG. 1D depicts existing techniques for a signal-analysis algorithm for use in flow analysis, in which a plurality of velocity calculations using imaging data are performed sequentially. In FIG. 1D, the reference to LS-PIV refers to a sequential calculation of velocity in one dimension only, as described in Kim T N, Goodwill P W, Chen Y, Conolly S M, Schaffer C B, Liepmann D, Wang R A. Line-scanning particle image velocimetry: an optical approach for quantifying a wide range of blood flow speeds in live animals. PLoS One. 2012;7(6):e38590. doi: 10.1371/journal.pone.0038590. Epub 2012 Jun. 26. PMID: 22761686; PMCID: PMC3383695, incorporated herein by reference.

FIG. 1E depicts an embodiment of a technique of the present invention for a signal-analysis algorithm for use in flow analysis, in which a plurality of velocity calculations using imaging data are performed concurrently, enabling obtaining flow analysis results substantially in real time. In FIG. 1E, RBC-PIV refers to an algorithm capable of parallelized execution of n-dimensional data, obtained from any contrastive modality, according to an embodiment of the present invention. An aspect of embodiments of signal-analysis algorithms for flow analysis of the present invention is concurrent processing. In embodiments flow analysis algorithms (i.e., signal-based analysis algorithms for flow analysis) of the present invention, flow analysis may be performed in parallel because each frame (i.e., images) contains information about an object's (such as, for example, a red blood cell or cluster of red blood cells; however, the present invention is not so limited) position in physical space. Aspects of such embodiments of algorithms, i.e., aspects of RBCPIV, which localizes objects in physical space, can do so concurrently because each frame is fundamentally independent from the next. Velocity calculations require depicting the same object displaced across time, but simply localizing the object in the frame does not require any information beyond what is contained in an individual frame. Embodiments of signal processing algorithms for flow analysis, i.e., embodiments of RBCPIV algorithms, processes a sequence of images independent of how the images were taken. That is, embodiments of signal processing algorithms for flow analysis, i.e., embodiments of RBCPIV algorithms, are not dependent on a specific technique for collecting images for conducting flow analysis based on such images and may be used for analysis of contrastive signal obtained from any imaging modality (including but not limited to the modalities of micro-CT, second-harmonic generation, third-harmonic generation, 2-photon and 3-photonmicroscopy (or still higher order processes) and confocal microscopy, for example). Further details demonstrating micro-CT being used in vessels can be found in: Zagorchev L, Oses P, Zhuang Z W, Moodie K, Mulligan-Kehoe M J, Simons M, Couffinhal T. Micro computed tomography for vascular exploration. J Angiogenes Res. 2010 March 5;2:7. doi: 10.1186/2040-2384-2-7. PMID: 20298533; PMCID: PMC2841094, the disclosure of which is incorporated herein by reference.

In some cases, concurrent processing may be achieved by utilizing any number of graphics processing units, such as commercially available graphics processing units, or other computer processing technologies capable of parallel processing. For example, the exemplary signal processing algorithm described above analyzes each pair of images concurrently, utilizing Graphics Processing Unit (GPU) cores from a commercially available NVIDIA GPU, and results in substantial execution speed increases.

### Applications:

As described above, any convenient structure may be imaged and/or manipulated, as the case may be, in connection with the methods, adaptors and systems of the present invention, and such may vary. Further, the structure may be imaged and/or manipulated through any convenient non-transparent tissue, such as ocular or periocular tissue, and such may vary.

As such, in some cases, the method of the present invention is a method of transscleral imaging. In other cases, the method of the present invention is a method of trans-conjunctiva imaging. In still other cases, the method of the present invention is a method of trans-Tenon's capsule imaging. In yet other cases, the method of the present invention is a method of extraocular muscle imaging. In certain cases, the method of the present invention is a method of imaging through the external palpebral tissue including one or more of dermis or muscle or aponeurosis. In some cases, the method of the present invention is a method of imaging through the internal palpebral tissue, including conjunctiva, tarsus, Meibomian glands or muscle. In other cases, the method of the present invention is a method of trans-orbital septum imaging. In still other cases, the method of the present invention is a method of trans-capsolupalpebral fascia imaging. In yet other cases, the method of the present invention is a method of trans-tarsus fascia imaging. In some cases, the method of the present invention is a method of trans-tarsal gland imaging. In other cases, the method of the present invention is a method of trans-periocular adipose tissue imaging. In still other cases, the method of the present invention is a method of trans-dermal imaging. In yet other cases, the method of the present invention is a method of imaging through pigmented uveal tissues. In some cases, the method of the present invention is a method of imaging through light-scattering tissue. In other cases, the method of the present invention is a method of imaging through light-absorbing tissue. In still other cases, the method of the present invention is a method of intravital imaging. In yet other cases, the method of the present invention is a method of quantification of fluid flow, such as blood flow, such as choroid blood flow. In some cases, the method of the present invention is a method of quantification of retinal blood flow. In other cases, the method of the present invention is a method of quantification of ciliary body blood flow. In still other cases, the method of the present invention is a method of quantification of uveal blood flow. In yet other cases, the method of the present invention is a method of quantification of conjunctival blood flow.

As described above, the present invention relates to applying multiphoton excitation to microscopy. As such, in some cases, the method of the present invention is a method of deploying multi-photon excitation microscopy on non-transparent tissue, such as ocular or periocular tissue. In other cases, the method of the present invention is a method of deploying multi-photon excitation microscopy through non-transparent tissue, such as ocular periocular tissue. In such embodiments, multi-photon excitation microscopy may comprise one or more of: two-photon excited fluorescence (2PEF) or second harmonic generation (SHG) or three-photon excited fluorescence (3PEF) or third harmonic generation (THG), four-photon excited fluorescence, fourth harmonic generation or other higher order multiphoton processes.

As described above, the present invention relates to imaging and/or manipulating, as the case may be, any convenient structure. As such, in some cases, the method of the present invention is a method of imaging and/or manipulating living tissue. It is contemplated that the embodiments of the present invention may be applied to any convenient living tissue. In some cases, the living tissue may be living ocular or living periocular tissue. In other cases, the living tissue may be dermatologic tissue or vascular tissue or neural tissue.

Other potential applications of imaging and/or manipulating tissue with embodiments of the present invention include, but are not limited to, imaging neuronal activity, imaging a corneal nerve, imaging aspects of a central nervus system, utilizing an invasive probe to image aspects of a central nervus system, thinning a region of a skull to image aspects of a central nervus system, visualizing nerve structures, utilizing imaged nerve structures to mitigate pain, utilizing an imaged structure to perform flow analysis, performing flow analysis substantially in real time, generating a velocity map of fluid flow within an imaged structure, diagnosing disease, diagnosing cancer, distinguishing between cancerous and non-cancerous tissues, distinguishing between cancerous and non-cancerous cells, preventing or treating retinal breaks, providing non-thermal treatment, providing non-thermal treatment to tumors, visualizing orbital fat, manipulating dermatologic tissue, imaging one or more of epidermis, dermis or hypodermis, manipulating a tear duct to facilitate fluid flow within the tear duct, mitigating ocular tissue redness, delivering therapy to an imaged structure, guiding positioning of an intraocular implant based on the imaged structure, predicting effective placement of intraocular implant based on the imaged structure, guiding positioning of an intraocular implant based on the imaged structure while implanting the intraocular implant, evaluating a position of an implanted intraocular implant based on the imaged structure, evaluating an effective lens position (ELP) of an implanted intraocular implant based on the imaged structure, detecting cancerous tissue, such as melanoma, providing cosmetic treatment, providing cosmetic dermatologic treatment, providing cosmetic surgery, treating scars, removing scars, treating acne scars, removing acne scars, treating skin discoloration, removing birthmarks, removing port-wine stains, treating skin discoloration disorders, removing tattoos, treating rosacea, providing controlled cutting of tissue, providing controlled cutting of dermatologic tissue, performing a skin biopsy procedure, disrupting cancerous tissue, disrupting one or more cancer cells, ablating cancerous tissue, ablating one or more cancer cells, ablating melanoma, ablating skin melanoma, removing hair, disrupting hair follicles, ablating hair follicle tissue, affecting tissue shape, affecting a shape of one or more fat deposits, reducing a volume of one or more fat deposits, reducing one or more subdural fat deposits, providing tissue sculpting, providing skin tightening, treating dry eye syndrome, mitigating eye redness, providing pain management or perforating tissue, for example.

Imaging and/or Manipulating Tissue of a Subject:

In some cases, the method of the present invention is a method of imaging and/or manipulating tissue, such as ocular or periocular tissue, of a subject. In such embodiments, the subject may have glaucoma or a retinal break or a choroidal tumor or similar condition or disease in the same tissue or tissue proximal thereto. In some cases, the method of the present invention is a method of imaging and/or manipulating dermatologic tissue of a subject. In such embodiments, the subject may have a dermatologic condition or feature, such as skin discoloration conditions, the presence of a nevus, a scar, or disease conditions, such as melanoma or other cancerous tissue. In such embodiments, the subject may seek to cosmetic treatment, such as skin tightening or a reduction in fat deposits or a reshaping of tissue. In embodiments, the subject is human and may be male or female of any age and with no specific medical history or history of disease or family history of disease. In embodiments, the subject is a human and has any degree of pigmentation of ocular tissue or periocular tissue or dermatologic tissue; i.e., such that the ocular or periocular tissue is non-transparent to any greater or lesser degree. In embodiments, the ocular or periocular tissue and/or the imaged structure comprise living tissue. In embodiments the subject is a human that is alive.

### Computer Implemented Embodiments:

Certain of the method and algorithm steps described in connection with the embodiments disclosed herein can be implemented as electronic hardware, computer software, or combinations of both. To clearly illustrate this interchangeability of hardware and software, various illustrative steps have been described above generally in terms of their functionality. Whether such functionality is implemented as hardware or software depends upon the particular application and design constraints imposed on the overall system applying a method according to the present disclosure. The described functionality can be implemented in varying ways for each particular application, but such implementation decisions should not be interpreted as causing a departure from the scope of the disclosure.

Certain illustrative steps, components, and computing systems (such as devices, databases, interfaces and engines) described in connection with the embodiments disclosed herein can be implemented or performed by a machine, such as a general purpose processor, a graphics processor unit, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. A computing environment can include any type of computer system, including, but not limited to, a computer system based on a microprocessor, a graphics processor unit, a mainframe computer, a digital signal processor, a portable computing device, a personal organizer, a device controller, and a computational engine within an appliance, to name a few.

Certain steps of a method, process, or algorithm described in connection with the embodiments disclosed herein can be embodied directly in hardware, in a software module executed by a processor, or in a combination of the two. A software module, engine, and associated databases can reside in memory resources such as in RAM memory, FRAM memory, flash memory, ROM memory, EPROM memory, EEPROM memory, registers, hard disk, a removable disk, a CD-ROM, or any other form of non-transitory computer-readable storage medium, media, or physical computer storage known in the art. An external storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

### Adaptors for Imaging, as Well as Manipulating, a Structure Through Non-Transparent Tissue

Aspects of the present disclosure include adaptors for use in practicing the methods of the present invention. In particular, the present disclosure includes adaptors for coupling an optical system to non-transparent tissue, such as ocular or periocular tissue. Adaptors of the present invention provide stabilization and physical coupling to tissue, such as ocular or periocular tissue. Specifically, the present disclosure includes adaptors for coupling an optical system to non-transparent tissue, such as ocular or periocular tissue, comprising: a first component configured to interface with an optical system configured to image or manipulate a structure through non-transparent tissue, and a second component connected to the first component and configured to interface with non-transparent tissue. Embodiments of adaptors of the present invention are utilized in connection with imaging a structure. Embodiments of adaptors of the present invention are utilized in connection with manipulating a structure (e.g., providing non-incisional therapy; or photo-tissue interactions, wherein, optionally, the photo-tissue interactions comprise laser-tissue interactions or laser-tissue perturbations; or multi-photon-mediated thermal damage; or non-thermal treatment; or photo-disruption; or blood vessel coagulation; or ablation).

Further embodiments of the invention comprise an immersion media for biological imaging comprising an immersion gel. In some cases, the immersion gel comprises a hyaluronan gel. In certain cases, the immersion gel comprises hyaluronic acid and deuterium oxide (heavy water). In other cases, the immersion gel is configured for imaging deep biological structures, e.g., tissues. In still other cases, the immersion gel is configured for use with long wavelength (e.g., 1,700 nm) lasers. In instances, the immersion gel is configured such that the immersion gel is an adaptor between an optical system and biological tissue (e.g., the immersion gel interfaces directly between aspects of an optical system and biological tissue). Methods of the present invention (i.e., methods for imaging a structure and methods for manipulating a structure) as described herein further comprise employing an embodiment of such an immersion gel. Immersion gels of the present invention are described in greater detail below.

Embodiments of adaptors comprise a coupling agent. Coupling agents of interest comprise transparent media, such as, for example, water or a viscous gel. In some cases, the coupling agent is present between the optical system and the ocular or periocular tissue, such as between the first component and the ocular or periocular tissue. In some cases, the coupling agent comprises a gel immersion media configured to increase a stability and duration of practicing the subject methods, e.g., imaging a structure or manipulating structure according to methods of the present invention. Embodiments of the present invention further comprise making and/or utilizing viscoelastic gel containing heavy water. Such component is of interest in in connection with embodiments for imaging at longer wavelengths (e.g., 1,700 nm) and in which normal water (not heavy water) absorbs much of these wavelengths.

In embodiments, a lens used to focus laser light into the tissue can be air immersion, water immersion, oil immersion or gel immersion. In certain embodiments, water immersion is preferred for multiphoton imaging. For example, for three-photon imaging at approximately 1,700 nm, water absorbs a significant amount of this light, and therefore, it may be preferable to use deuterium oxide (heavy water) as an immersion fluid at this wavelength. In certain cases, using viscoelastic gel with water-immersion objectives may be preferable to using water in certain imaging contexts (e.g., as the traditional use of water in certain imaging contexts is known in the art). In some cases, using a transparent gel for an immersion media is preferred. In embodiments, transparent gels can make the optical interface easier and much more stable, and in some instances, obviate the need for a coupling device altogether. Transparent gels of interest include viscoelastic gel, such as a viscoelastic gel designed for intraocular surgery.

In embodiments of adaptors of the present invention, the second component is configured to interface with tissue, such as ocular or periocular tissue, using suction. In some cases, the second component comprises a suction mechanism for attaching the second component and the tissue, such as the ocular or periocular tissue.

In embodiments, of adaptors of the present invention, the second component comprises a central portion. In some cases, the central portion is hollow. In some cases, the hollow central portion receives fluid. In other cases, the central portion is solid. In some cases, the solid central portion is optically transparent. In some cases, the second component comprises an interface surface, wherein the interface surface contacts the non-transparent tissue, such as ocular or periocular tissue. In such cases, the interface surface may interface with an eye or with ocular tissue. In some cases, the interface surface is shaped to contact an eye or ocular tissue. In some cases, the interface surface is shaped to be displaced relative to the cornea or sclerocorneal limbus. That is, there may be a recess or cut-out in the interface surface to accommodate the raised cornea or the shape of the sclerocorneal limbus. For example, in embodiments, the interface surface is shaped to accommodate placement on a section of a cornea or sclerocorneal limbus. In other embodiments, a section of the interface surface is depressed to accommodate placement on a cornea or sclerocorneal limbus. Embodiments of the present invention are not limited to imaging and/or manipulating structures exclusively through non-transparent tissue. In embodiments, the adapter may interface with the cornea, such as being present over the central cornea.

In embodiments, the second component is configured to interface with periocular tissue. In some cases, the second component interfaces with the conjunctival fornix. In other cases, the second component comprises a shape to interface with the conjunctival fornix. In some cases, the second component comprises a shape to fit between an eye and a lower eyelid of the eye. In other cases, the second component comprises a shape to fit between an eye and an upper eyelid of the eye.

In embodiments of adaptors of the present invention, the second component comprises a contact lens interface. In some cases, the first component translates relative to the non-transparent tissue, such as ocular or periocular tissue. In some cases, the first component allows the optical system to translate relative to the second component. In other cases, the first component rotates relative to the non-transparent tissue, such as ocular or periocular tissue. In some cases, the first component allows the optical system to rotate relative to the non-transparent tissue, such as ocular or periocular tissue. In some cases, the first component articulates relative to the non-transparent tissue, such as ocular or periocular tissue. In some cases, the first component allows the optical system to articulate relative to the non-transparent tissue, such as ocular or periocular tissue. In certain cases, the adaptor further comprises a mechanism to control translation or rotation or articulation of the optical system relative to the non-transparent tissue, such as ocular or periocular tissue. Any convenient device capable of translation, rotation, articulation may be applied. For example, commercially available stepper motors or servo motor and controls may be applied. Embodiments of adapters of the invention are configured to interface with dermatologic tissue, such as a skin surface. Embodiments of adapters of the invention are configured to interface with tissue such that the optical system may be configured to perform dermatologic procedures or provide dermatologic therapies or perform cosmetic therapies or treatments, such as, for example, tissue reshaping, debulking of fat deposits, skin tightening or addressing skin coloration issues or the like.

In some cases, adaptors of the present invention further comprise a coupling agent, where the coupling agent is present at an interface between the adaptor and the tissue, such as ocular or periocular tissue. Coupling agents of interest comprise any convenient biocompatible material, such as liquids or gels. Adaptors according to the present invention may be sterile or have a sterile surface. Adaptors according to the present invention may be disposable or may be reusable, in whole or in part.

In other embodiments of adaptors of the present invention, such as embodiments of adaptors for use in connection with gene or cell or drug therapies, the adaptor may comprise an injector for injecting an agent into the structure. In some cases, adaptors of the present invention comprise a needle injector. In other cases, adaptors of the present invention comprise a defined path, such as a hole through, or cut out from, the adaptor, for an injector to be positioned such that the location of the injection site of the injector may be precisely known.

### Exemplary Embodiments of Adaptors:

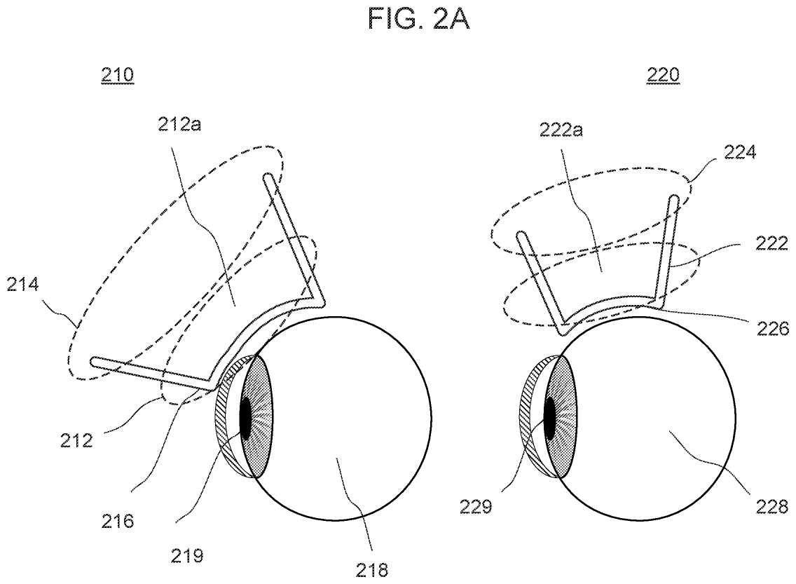

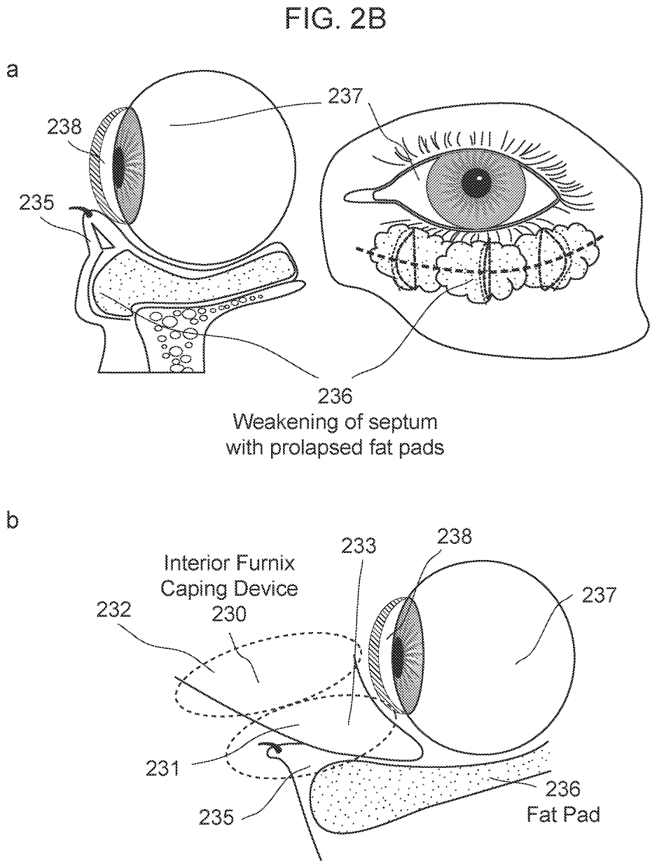

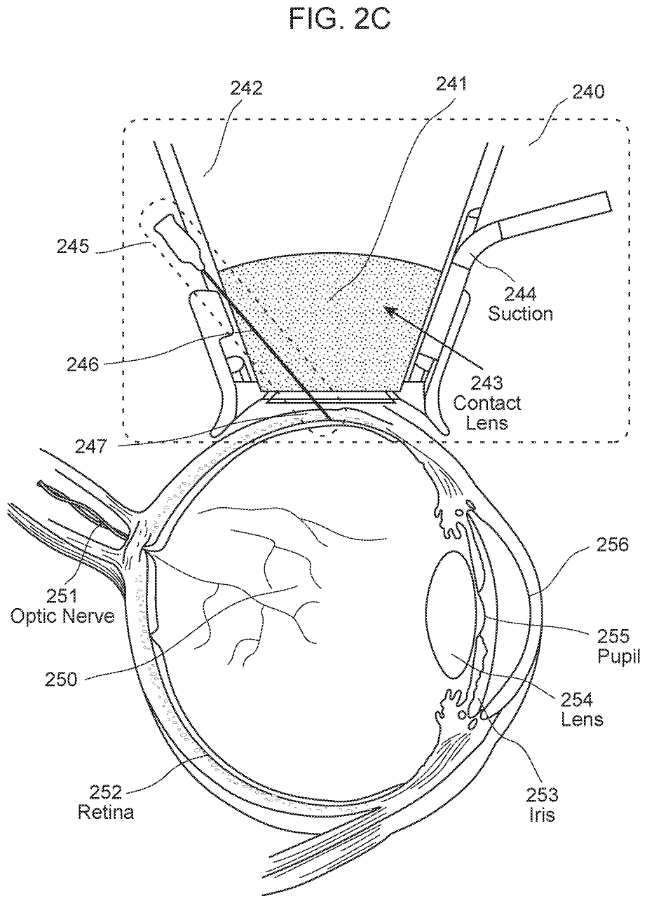

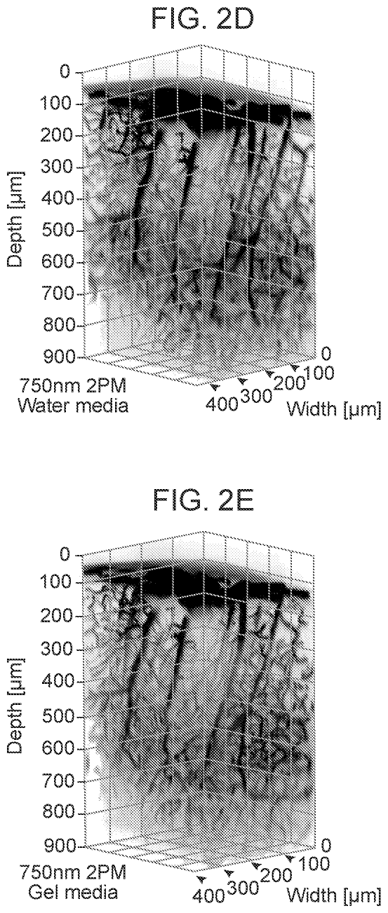

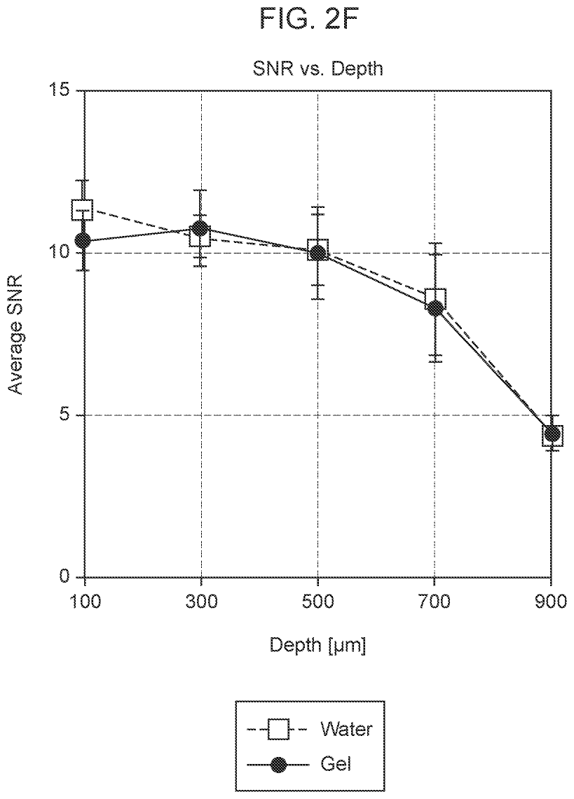

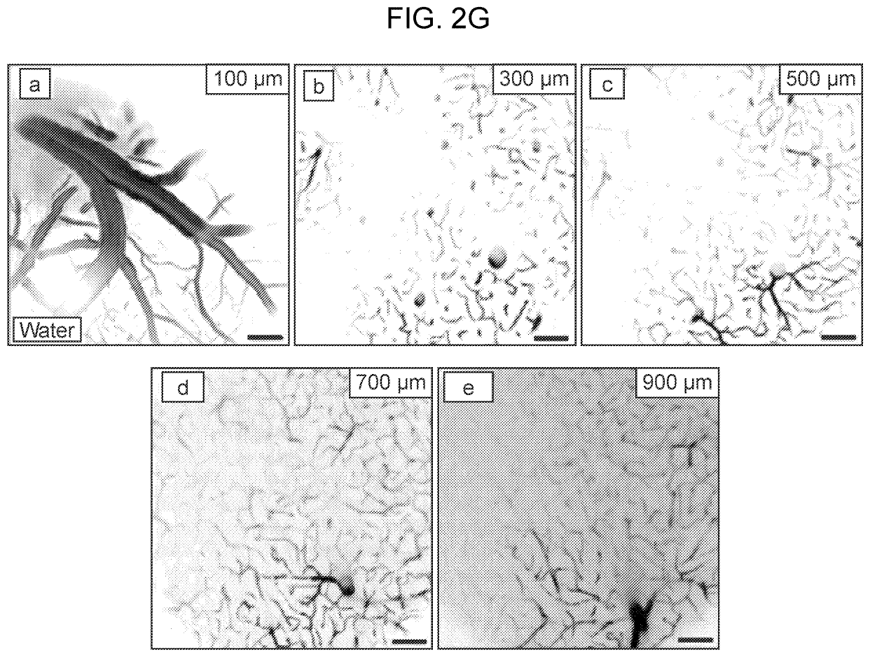

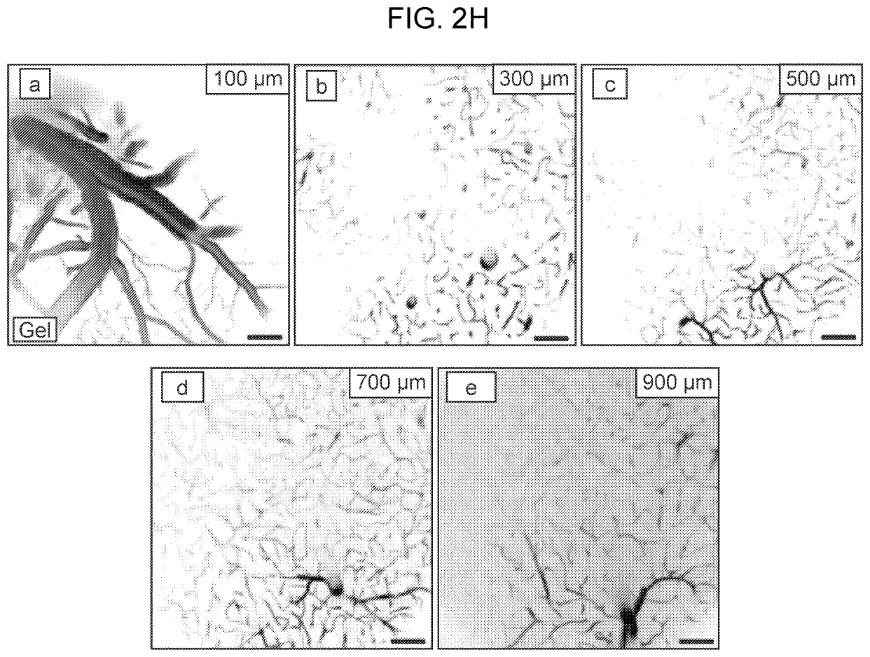

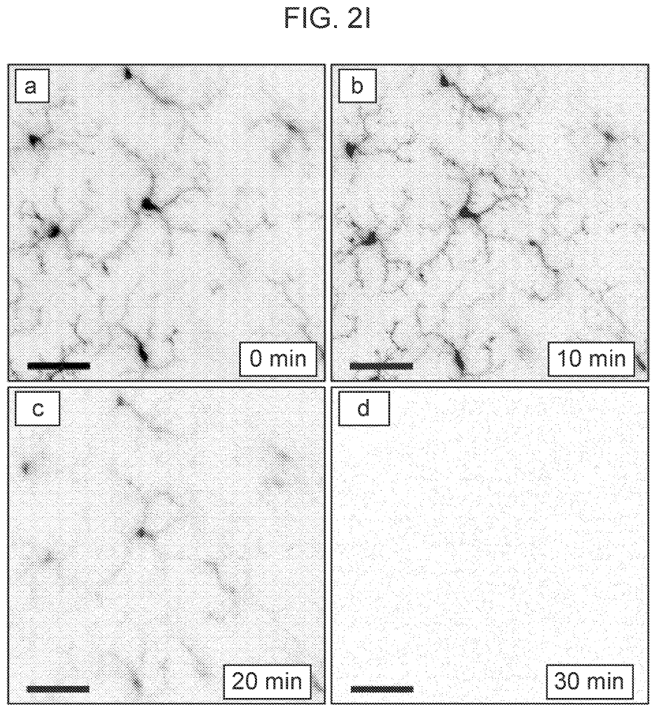

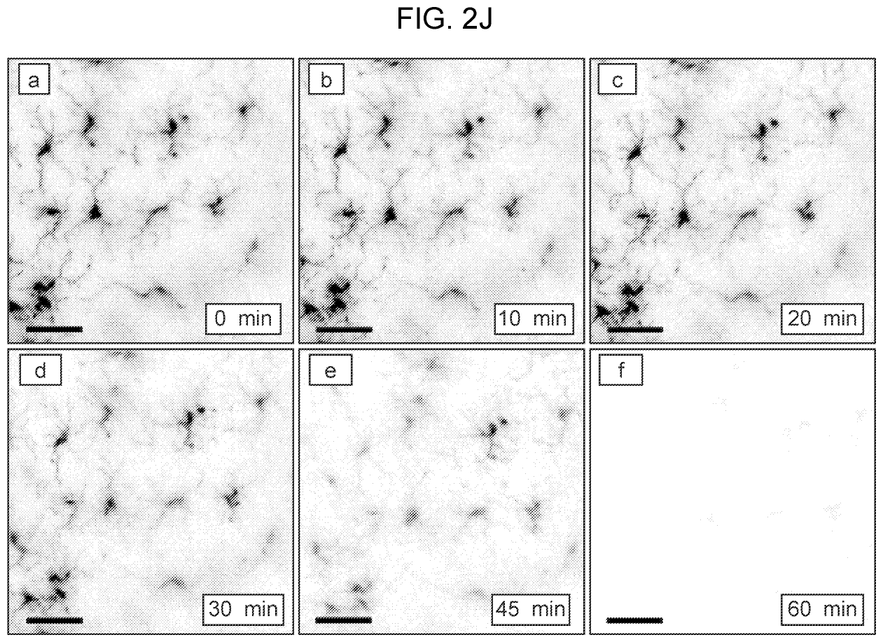

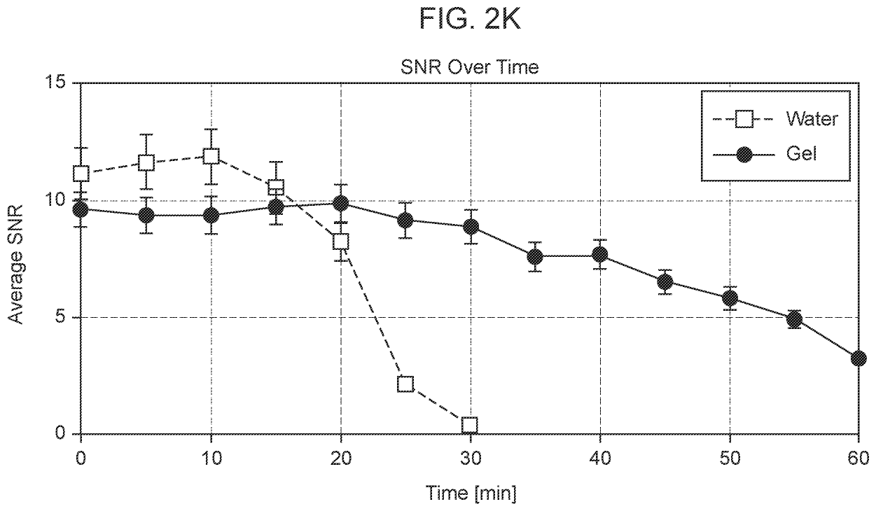

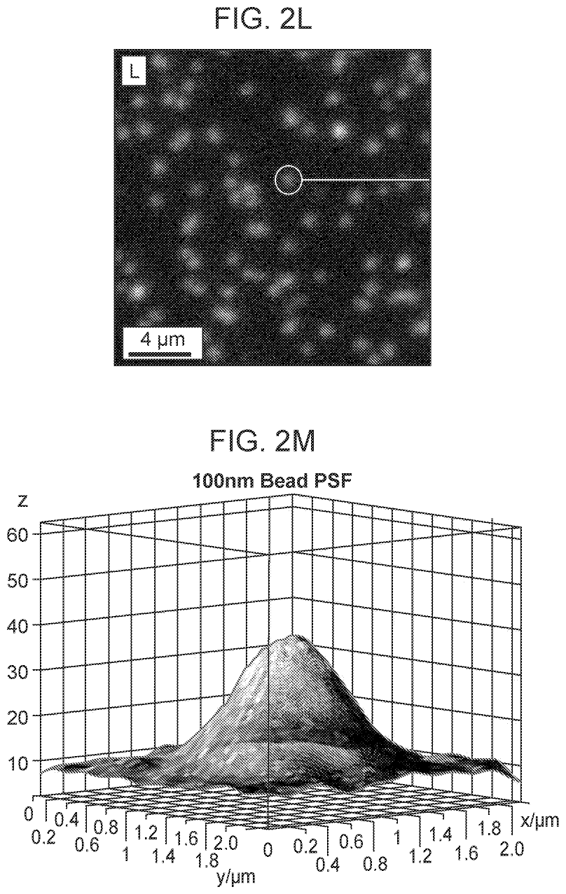

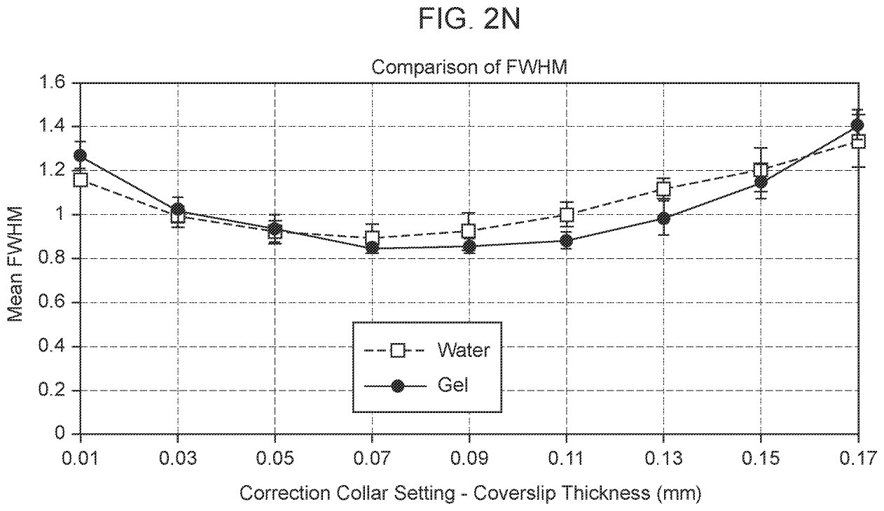

FIG. 2A illustrates first adaptor 210 for coupling an optical system to non-transparent ocular tissue according to an embodiment of the present invention as well as second adaptor 220 for coupling an optical system to a different aspect of non-transparent ocular tissue according to an embodiment of the present invention. Embodiments of adaptors 210 and 220 of the present invention depicted in FIG. 2A relate to adaptors for imaging or manipulating any convenient structure accessible through non-transparent ocular tissue according to the present techniques and such may vary. Similarly, embodiments of adaptors 210 and 220 of the present invention depicted in FIG. 2A relate to adaptors for interfacing with any convenient optical system and such may vary. Adaptors 210 and 220 are exemplary embodiments of the present invention provided for illustrative purposes, and aspects thereof may vary as desired in embodiments of the present invention.

As seen in FIG. 2A, adaptor 210 comprises second component 212 that interfaces directly with non-transparent ocular tissue 218. In particular second component 212 of adaptor 210 comprises a surface that is shaped to interface directly with an ocular tissue surface 218, i.e., a surface of an eye. Second component 212 comprises cut-out or recess or depression 216 which is shaped to accommodate the shape of the raised cornea 219 on ocular tissue surface 218. Adaptor 210 also comprises first component 214, located proximally to second component 212. First component 214 is shaped to receive an optical system (not shown in FIG. 2A) for imaging a structure (not shown in FIG. 2A) through the non-transparent ocular tissue 218. Adaptor 210 comprises central portion 212a that is an optically transparent solid.

Also seen in FIG. 2A, adaptor 220 comprises second component 222 that interfaces directly with non-transparent ocular tissue 228. In particular second component 222 of adaptor 220 comprises a surface that is shaped to interface directly with an ocular tissue surface 228, i.e., a surface of an eye. Unlike second component 212 of adaptor 210, second component 222 is shaped to interface with an ocular tissue surface 228 that does not include raised cornea 229, and therefore surface 226 of second component 222 comprises a smooth continuous surface without a cut-out or recess or depression. Adaptor 220 also comprises first component 224, located proximally to second component 222. First component 224 is shaped to receive an optical system (not shown in FIG. 2A) for imaging a structure (not shown in FIG. 2A) through the non-transparent ocular tissue 228. Adaptor 220 comprises central portion 222a that is an optically transparent solid.

FIG. 2B subpanel (a) shows the periocular fat pad 236 present within non-transparent periocular tissue 235, i.e., a lower eyelid 235, of eye 237 with raised cornea 238, responsible for bags under eyes, motivating the configuration of third adaptor 230 shown in FIG. 2B subpanel (b).

FIG. 2B subpanel (b) illustrates third adaptor 230 for coupling an optical system to non-transparent periocular tissue 235 according to an embodiment of the present invention. Embodiments of adaptor 230 of the present invention depicted in FIG. 2B subpanel (b) relate to adaptors for imaging any convenient structure accessible through non-transparent periocular tissue 235 according to the present techniques and such may vary. Similarly, embodiments of adaptor 230 of the present invention depicted in FIG. 2B subpanel (b) relate to adaptors for interfacing with any convenient optical system and such may vary. Adaptor 230 is an exemplary embodiment of the present invention provided for illustrative purposes, and aspects thereof may vary as desired in embodiments of the present invention.

As seen in FIG. 2B subpanel (b), adaptor 230 comprises second component 231 that interfaces directly with non-transparent periocular tissue 235. In the embodiment shown, second component 231 is shaped to fit between a lower eyelid 235 of eye 237. In particular, second component 231 comprises a wedge-like shape with either side of the wedge contacting periocular tissue comprising the lower eyelid 235 and the eye 237. Further, in the embodiment shown, second component 231 is shaped such that it can access fat pad 236 through periocular tissue 235. In the embodiment shown, the optical system may be configured to image such periocular tissue 235 as well as manipulate such periocular tissue 235, e.g., via providing non-incisional therapy, photo-tissue interactions, multi-photon-mediated damage, such as thermal damage, photo-disruption, photo cross-linking, blood vessel coagulation, ablating tissue or the like. Second component 231 may, but need not, provide a surface with a cut-out or recess or depression (not shown in FIG. 2B subpanels (a) or (b)), which is shaped to accommodate the shape of the raised cornea 238 on ocular tissue surface of eye 237. Adaptor 230 also comprises first component 232, located proximally to second component 231. First component 232 is shaped to receive an optical system (not shown in FIG. 2B subpanels (a) or (b)) for imaging a structure, such as fat pad 236 through non-transparent periocular tissue 235.

Adaptor 230 comprises central portion 233 that is an optically transparent solid. FIG. 2C illustrates fourth adaptor 240 for coupling an optical system to non-transparent ocular tissue 250 according to an embodiment of the present invention. Fourth adaptor 240 illustrates an embodiment comprising a needle injector 245 for use with gene or cell or drug therapies. Embodiments of adaptor 240 of the present invention depicted in FIG. 2C relate to adaptors for imaging, as well as accessing via needle injector, any convenient structure accessible through non-transparent ocular tissue 250 according to the present techniques and such may vary. Similarly, embodiments of adaptor 240 of the present invention depicted in FIG. 2C relate to adaptors for interfacing with any convenient optical system (not shown in FIG. 2C) and such may vary. Adaptor 240 is an exemplary embodiment of the present invention provided for illustrative purposes, and aspects thereof may vary as desired in embodiments of the present invention.

As seen in FIG. 2C, adaptor 240 comprises second component 241 that interfaces directly with non-transparent ocular tissue 250. In the embodiment shown, second component 241 is shaped to interface with, i.e., rest on the surface of, a top surface of ocular tissue 250 comprising optic nerve 251, retina 252, iris 253, lens 254, pupil 255 as well as cornea 256. In particular, the interface surface of second component 231 comprises a substantially curved shape corresponding with the curved shape of ocular tissue 250, in particular, a top surface of ocular tissue 250. In the embodiment shown, an optical system may be configured to image aspects of ocular tissue 250 as well as manipulate such ocular tissue 250, e.g., via providing non-incisional therapy, photo-tissue interactions, multi-photon-mediated damage, such as thermal damage, photo-disruption, photo cross-linking, blood vessel coagulation, ablating tissue or the like. Second component 231 may, but need not, provide a surface with a cut-out or recess or depression (not shown in FIG. 2C), which is shaped to accommodate the shape of a raised cornea 256 on a surface of ocular tissue 250. Second component 241 comprises contact lens 243 for interfacing with ocular tissue 250 as well as optically adjusting light transmitted to, or light detected from, non-transparent ocular tissue 250. Adaptor 240 also comprises first component 242, located proximally to second component 241. First component 242 is shaped to receive an optical system (not shown in FIG. 2C) for imaging a structure, such as aspects of ocular tissue 250, including, for example, aspects of optic nerve 251, retina 252, iris 253, lens 254, pupil 255 or cornea 256, through non-transparent ocular tissue 250.

Adaptor 240 further comprises suction mechanism 244 for attaching adaptor 240 to surface of ocular tissue 250. Suction mechanism 244 comprises a hollow tube fluidically connecting an interface volume between second component 241 and surface of ocular tissue 250, such that applying a low pressure source to the suction mechanism 244 has the effect of drawing adaptor 240 to ocular tissue 250 and sealing adaptor 240 thereto while the low pressure source is applied to suction mechanism 244.

Adaptor 240 further comprises needle injector 245 for use in connection with applying gene or cell or drug therapy. Needle injector 245 comprises needle 246 with injection tip 247, i.e., a tip of needle out of which fluid may be delivered. Needle injector 245 is integrated into adaptor 240 such that needle 246 as well as injection tip 247 may be positioned in one or more known locations relative to the imaged structure (i.e., one or more known locations of the imaging field of view of an optical system (not shown in FIG. 2C) attached to first component 242 of adaptor 240, such as the center of the field of view at various, configurable depths therefrom). That needle injector 245 is present in one or more fixed locations relative to the imaged structure enables the delivery of one or more agents for gene or cell or drug therapy to be delivered to a precise location of ocular tissue 250, thereby facilitating more specific or targeted gene or cell or drug therapy, i.e., such that an agent is delivered to a desired location and, substantially, not delivered to a location other than the desired location.

As described above, embodiments of the present invention comprise utilizing an immersion media in connection with an embodiment of an adaptor. In embodiments, viscoelastic gel may be used instead of water as a viscous alternative immersion media in order to help mitigate visual aberrations produced by motion artifact. Further, certain embodiments of viscoelastic gel do not require an adaptor (e.g., an adaptor as depicted in FIGS. 2A-C) (in some cases such an element may be referred to as a reservoir and in certain contexts such element is configured to function as a reservoir (a reservoir of immersion media), i.e., such that embodiments of viscoelastic gel, when used in optical systems, such as those described herein, do not require use of such an adaptor and/or reservoir) to remain between the sample and aspects of the optical system, such as the objective, and may provide equivalent if not superior imaging quality for in vivo applications (i.e., as compared with embodiments that do require an adaptor and/or reservoir). In addition, viscoelastic gel may help stabilize the focus between aspects of the optical system, such as the objective, and a sample, i.e., the structure, during long-term imaging or manipulation of the structure, as applicable, allowing for superior image capturing or manipulation of the structure in the context of timelapses.

Clear viscous gels of embodiments of the present invention may be used as an alternative immersion medium to water. Such viscous gels partially reduce distortion caused by motion artifact, evaporate at a slower rate than water, do not require reservoirs like those found on cranial windows, and have a refractive index (ri 1.3365) that is close enough to water (ri 1.333) to be compatible with water-immersion objectives. In embodiments with viscous gels with variable formulations of 0.5%-4%, the refractive index is typically expected to be within a range of about 1.335-1.350. In embodiments, aspects of optical systems, such as, for example, objectives or lenses may be specifically designed for a desired immersion medium that is a viscous gel. That is, embodiments of the present invention comprise aspects of an optical system, such as lenses or objectives or other components of optical systems, configured for use (e.g., tailored to compensate for effects of using such embodiment of an optical system) with a viscous gel, such as a viscous gel described herein.

Embodiments of viscoelastic gels of the invention comprise hyaluronic acid. Certain embodiments of viscoelastic gels of the invention comprise hyaluronic acid and water. Hyaluronic acid is relatively cheap and a non-clinical version of this gel may be a satisfactory. Other embodiments of viscoelastic gels further comprise other ingredients, such as, for example, deuterium oxide (heavy water), chondroitin sulfate and hydroxypropyl methylcellulose. Embodiments of viscoelastic gels of the invention are highly customizable in their formulation when using variable concentrations of hyaluronic acid or other viscosity promoting substances, such as, for example, chondroitin sulfate. Other embodiments of viscoelastic gels of the invention comprise aspects, i.e., ingredients, that promote viscosity and/or promote high quality imaging. Modifying gel composition may produce a more viscous gel with improved utility in long-term imaging and surgical procedures with poor surgical and/or optical access. While these modifications may result in a greater difference in refractive index from water, embodiments of the present invention can correct for this effect using a correction-collar that adjusts aspects of the optical system, such as the objective's internal optical components. Moreover, other aspects of the optical system, such as new objectives, may be specially designed to optimize compatibility with embodiments of viscous gels, further improving imaging quality and stability. Embodiments utilizing three-photon imaging using water-immersion objectives may require heavy water (deuterium oxide) to avoid the absorption that occurs at longer infrared laser wavelengths, usually past 1,700 nm. As embodiments of viscoelastic gels may be composed primarily of hyaluronic acid and water, heavy water may be used to replace regular water in a new formulation, allowing for compatibility with both two-and three-photon laser microscopy.

Embodiments of viscoelastic gels containing hyaluronic acid (HA) that are optimized for imaging purposes may have concentrations between 0.1% and 10% (% weight/volume) in water. The molecular weight (MW) of HA polymers can range from 100,000 Daltons to 20,000,000 Daltons. The concentration and MW can be adjusted in embodiments to fine-tune viscoelastic properties and improve its utility in various imaging contexts. Additionally, including chondroitin sulfate (CS) in embodiments at concentrations of between 0.1 and 10% with a MW between 1,000 Daltons and 1,000,000 Daltons may be used to further alter the viscoelastic properties of such gels.

FIGS. 2D-H present a comparison of imaging quality when using water versus a gel immersion media according to an embodiment of the present invention in the context of a standard mouse cranial window. FIGS. 2D-E show a 900 μm z-stack taken of mouse cortical vasculature stained with Texas-Red and using water (FIG. 2D) and gel (FIG. 2E) with a 25× water-dipping objective. FIG. 2F show how results of the average signal to noise ratio (SNR) (calculated as 10*log(average signal/noise) follows approximately the same trend when using water or a gel according to an embodiment of the present invention to image a depth of up to 900 μm. FIGS. 2G-H present normalized cross-sections at 100 μm of depth in FIG. 2G subpanel (a) and FIG. 2H subpanel (a), 300 μm of depth in FIG. 2G subpanel (b) and FIG. 2H subpanel (b), 500 μm of depth in FIG. 2G subpanel (c) and FIG. 2H subpanel (c), 700 μm of depth in FIG. 2G subpanel (d) and FIG. 2H subpanel (d), and 900 μm of depth in FIG. 2G subpanel (e) and FIG. 2H subpanel (e), showing approximately equivalent imaging quality with water (FIG. 2G subpanels (a)-(e)) and a gel immersion media according to an embodiment of the present invention (FIG. 2H subpanels (a)-(e)).

FIGS. 21-K present experimental results showing improved stability of immersion media during long-term in vivo imaging when using a gel according to an embodiment of the present invention. In FIGS. 21-K, microglia tagged with enhanced Green Fluorescent Protein (eGFP) were imaged in the mouse retina to produce timelapses of at least 30 minutes. FIG. 21 shows initial image quality when using water at FIG. 21 subpanel (a) and then after 10 minutes at FIG. 21 subpanel (b). FIG. 21 shows how image quality when using water alone degrades significantly at 20 min (i.e., FIG. 21 subpanel (c)) until there is virtually no signal at 30 min (i.e., FIG. 21 subpanel (d)). FIG. 2J shows initial image quality when using a gel according to an embodiment of the present invention at FIG. 2J subpanel (a) and then after 10 minutes at FIG. 2J subpanel (b). FIG. 2J shows how image quality when using a gel according to an embodiment of the present invention remained roughly constant for the first 20 minutes (i.e., FIG. 2J subpanel (c)) and then degraded at a much slower rate with FIG. 2J subpanel (d) showing image quality at 45 min, until becoming unusable at approximately 60 min (i.e., FIG. 2J subpanel (f)). FIG. 2K shows that average SNR (calculated as 10*log(average signal/noise)) for the timelapse depicted in FIGS. 21-J can be seen to initially lower for the gel according to an embodiment of the present invention but is more stable over the hour of constant in vivo imaging and is superior to water after the 15 minute mark.

FIGS. 2L-N present results showing how reduced image quality from using a gel that is an embodiment of the present invention can be ameliorated using an optical system comprising a correction collar that adjusts for refractive index differences. The correction collar settings are given as a range of thicknesses of hypothetical glass coverslips placed between the sample (i.e., the non-transparent tissue) and the optical system (i.e., the objective), ranging from 0.01 mm to 0.17 mm, that can be corrected for via adjustments of internal optical components. FIG. 2L shows a Z-projection of several 100 nm fluorescent beads. FIG. 2M shows a 3-D radial point spread function (PSF) of the red-circled bead in FIG. 2L. Point spread function (PSF) may be used to help measure imaging quality of an optical system by showing a 3D distribution of a signal produced by a so-called “infinitely small point.” In this case, the “infinitely small points” are several 100 nm fluorescent beads each producing signals roughly 1 μm wide in FIG. 2L. The actual PSF is the 3D distribution shown in FIG. 2M.

FIG. 2N shows that at evenly spaced correction collar setting from 0.01 mm to 0.17 mm, 5 random beads were selected and their PSFs analyzed to obtain the mean resolution (calculated as full width at half maximum (FWHM) in μm) at each correction collar setting. Full width at half maximum (FWHM) is the width of the PSF at half of its maximum value. This parameter is commonly used to describe spatial resolution, in which a smaller FWHM indicates better resolution. Initially water outperformed gel; however, from 0.07 mm to 0.15 mm the FWHM when using gel is lower, suggesting that any impairments in imaging quality when using gel can be corrected for and even result in superior resolution over water. The correction collar settings are given as a range of thicknesses of glass coverslips placed between the sample and the objective, ranging from 0.00 mm (no coverslip) to 0.17 mm, that can be corrected for via adjustments of internal optical components.

For the embodiments shown and/or reported on in FIGS. 2D-N, the embodiment of the gel immersion media used was a viscoelastic gel containing 1% sodium hyaluronate (sodium hyaluronate is the non-hydrated/powder form of hyaluronic acid; i.e., sodium hyaluronate and hyaluronic acid are otherwise the same compound) with an average molecular weight of 2,500,000 Daltons, dissolved in physiological phosphate-buffered saline (PBS). Specifically the formula is: 10 mg sodium hyaluronate, 0.45 mg sodium phosphate, 7.5 mg sodium chloride, all dissolved in 1 mL of water. This formulation uses PBS as the base, which comprises all those salts, and is designed for procedures such as, for example, cataract surgeries. However, in other embodiments, water alone can be used instead of PBS, and the formulation can be optimized for imaging (i.e., by using different concentrations, molecular weights, and other potential ingredients, such as, for example, heavy water and chondroitin sulfate). Further details regarding alternative formulations of viscoelastic gels are found in Kaur K, Gurnani B. Viscoelastics. [Updated 2023 Jun 11]. In: StatPearls [Internet]. Treasure Island (FL): StatPearls Publishing; 2023 Jan-. Available from: https://www.ncbi.nlm.nih.gov/books/NBK578189/, incorporated herein by reference.

### Systems for Imaging, as Well as Manipulating, a Structure Through Non-Transparent Tissue

Aspects of the present disclosure include systems for use in practicing the methods of the present invention. In particular, the present disclosure includes systems for imaging and/or manipulating a structure through non-transparent tissue, such as ocular or periocular tissue. Specifically, the present disclosure includes systems for imaging and/or manipulating a structure through non-transparent tissue, such as ocular or periocular tissue, comprising: an optical system configured to image and/or manipulate a structure through non-transparent tissue, an adaptor configured to couple the optical system to the non-transparent tissue, as such adaptors are described herein, a processor comprising memory operably coupled to the processor, wherein the memory comprises instructions stored thereon, which, when executed by the processor, cause the processor to: instruct the optical system to image a structure through the non-transparent tissue, receive information from the optical system about light emitted from the structure, and combine information about light emitted from the structure to generate an image the structure, and an operable connection between the processor and the optical system. Alternatively, in some cases, the system comprises a processor comprising memory operably coupled to the processor, wherein the memory comprises instructions stored thereon, which, when executed by the processor, cause the processor to: instruct the optical system to manipulate a structure through the non-transparent tissue or instruct the optical system to image and manipulate a structure through the non-transparent tissue.

In embodiments of systems of the present invention, any convenient commercially available optical system, or aspects thereof, may be employed, such as, for example, a microscope equivalent to Bergamo II Series, ThorLabs and/or a femtosecond laser for excitation equivalent to Insight X3, Spectra-Physics. Aspects of interest of optical systems comprise: (1) longer wavelength applications (e.g., 1,700 nm) may require special coatings on the optics to direct this range of light efficiently; (2) embodiments employ novel fast laser scanning techniques to increase speed of imaging dramatically; such aspects comprise modular modifications of multiphoton microscopes.

In certain embodiments, “two-photon microscopes” or “multiphoton microscopes” may be employed. For light sources, such as, for example, lasers, commercially available femtosecond lasers may be employed in certain embodiments, such as, for example, any of the Mira or Chameleon models by Coherent and the Octavius-2P Laser commercially available from ThorLabs, for example. In other cases, such lasers do not encompass the critical longer wavelengths needed for three-photon imaging. An example of a laser that does encompass such longer wavelengths includes Monaco 1035 (60W), which pumps the OPA. Such a setup is tunable between 650-2500 nm. In embodiments, the Monaco pump laser can be used for ablation and/or cutting applications.

Aspects of interest of commercially available lasers for use in embodiments utilizing three-photon imaging are configured to emit longer wavelength light, e.g., 1,700 nm or 1,800 nm light. Aspects of interest of commercially available lasers for use in embodiments of the present invention comprise the following described with respect to a Monaco 1035-80-60, Series II Pump laser, where Monaco 1035 is an industrial femtosecond laser with a MOPA architecture. Such is designed for high-uptime in 24/7 applications, and the laser family provides >80 μJ/pulse at 1035 nm. Standard repetition rates up to 50 MHz at 60 W enable current and future throughput requirements in materials processing and microelectronics applications. Homogeneous materials such as glass and metals, as well as complex, layered structures for the FPD and mobile markets are readily addressed with Monaco's sub-350 fs pulsewidth. Features of interest of such comprise: 80 μJ/pulse for processing of high ablation threshold materials; 60 W average power for high throughput; <350 fs standard pulsewidth; Variable pulsewidth from <350 fs to >10 ps for process tailoring; Repetition rate to 50 MHz for fast processing with polygon scanners; >320 UJ seeder burst mode; Compact single box design for ease of integration; Center Wavelength: 1035±5 nm; Output Power: 60 W; Energy: 80 uJ (up to 750kHz); Seeder Burst Mode >320 μJ; Repetition Rate: Single-shot to 50 MHz; Outbound AOM can pulse-pick from single-shot to 1 MHz; MÇ<1.2; Pulsewidth<350 fs; Pulsewidth tuning range<350 fs to >10 ps; Beam circularity>85%; Polarization Ratio>100:1; Polarization vertical±3°; Beam Pointing Stability<25 urad/C; Pulse Energy Stability<1.5% rms; Laser head weight: 110 lbs; External Comms: RS-232, Ethernet, USB; Power Consumption: 48VDC, <500 W; Operating Temperatures: Laser Head: 10-30 C, Power Supply: −20-60 C, Non-operation (storage): 5-65° C., Laser head length: 668.7 mm, Laser head width: 360.3 mm. Opera-F is an Optical Parametric amplifier (OPA) used to extend the turning range of the Coherent Monaco Yb amplifier system. Tt can extend the tuning capability of the Monaco from 650-900 nm (signal) and 1200-2500 nm (idler). Opera-F system combines the short pulse generation delivered from a non-collinear OPA with the broad turning range and ease of use of a collinear OPA. Opera-F incorporates a white-light seeded non-collinear pre-amplifier followed by a collinear power amplifier the highest levels of performance. Computer-controlled tuning; Standard tuning range 650-900 nm (signal) and 1200-2500 nm (idler); >10% conversion efficiency from pump (signal+idler); Optional compressors to deliver 25-75 fs pulses (signal) and 40-100 fs pulses (idler); Compatible with Monaco amplifier. In addition, of interest are commercially available units from Coherent that include a fixed 1300 nm 3P laser. Such commercial options may be available from Coherent, available at: https://www.coherent.com/lasers/ultrashort-pulse/monaco.

In embodiments, the optical system comprises a multi-photon excitation microscopy system. In some cases, the multi-photon excitation microscopy system comprises: an excitation source to emit light energy; and a detector to sense light emitted from the structure via multiphoton excitation. That is, optical systems of interest comprise an excitation source, e.g., one or more laser or other excitation source, as described in detail herein, as well as a detector, e.g., one or more photomultiplier tubes (PMT) or silicon photomultipliers and avalanche photodiodes or the like, for use in detecting light stimulated in the structure to be imaged. The optical system may comprise one or more laser scanners for rapid imaging or targeting the laser for treatment of tissue or treatment of the structure. The optical system may comprise a tube lens and a scan lens. Dispersion compensation may be employed to correct for chromatic dispersion and increase efficiency of signal generation or treatment of the tissue. Adaptive optical correction may be employed to improve resolution and/or to increase efficiency of signal generation or treatment of tissue. Power attenuation control may be employed by a number of methods, e.g., by an acousto-optic modulator, a Pockel Cell, or paired and rotating polarizers. In embodiments, the excitation laser is focused into the tissue with a high numerical aperture optic or objective lens. In some cases, the optical system and/or the adaptor comprises a translation stage controllable by the processor for moving the excitation source and/or detector of the optical system over a specified area or volume or direction in order to stimulate light in the structure to be imaged over a desired area or volume. In certain embodiments, the optical system adjusts focus in a Z-axis mechanically. For example, such an embodiment may employ a translation stage or piezoelectric mechanism, such as a piezoelectric actuator, for adjusting focus of in a Z-axis. In other embodiments, the optical system may employ optical scanning in a Z-axis in order to adjust focus in a Z-axis. Such optical scanning approach may offer less latency in adjusting focus, which may be preferred in certain clinical or experimental applications. In embodiments, commercially available components of optical systems may be employed. For example, certain embodiments employ one or more of: a high numerical aperture objective lens, such as N25X-APO-MP made by Nikon and commercially available from ThorLabs; a photomultiplier tube (PMT), such as PMT2100 commercially available from Thorlabs; a rotating polarizer, such as, BCM-PA variable attenuator commercially available from ThorLabs; a pockel cell, such as BCM-PCA pockel cell attenuator commercially available from ThorLabs; a piezoelectric actuator, such as PFM450-E commercially available from ThorLabs.

In some embodiments, the memory further comprises instructions, which, when executed by the processor, cause the processor to: instruct the optical system to image a structure over a specified volume. In other cases, the memory further comprises instructions, which, when executed by the processor, cause the processor to: instruct the optical system to translate or rotate or articulate relative to the non-transparent tissue, such as ocular or periocular tissue. In still other cases, the memory further comprises instructions, which, when executed by the processor, cause the processor to: instruct the optical system to image a structure over a specified time period. Any convenient time intervals may be employed, such as, for example, times between fractions of a second to potentially hours. In some cases, a structure is imaged for one or more seconds to one or more hours. In embodiments, structural imaging and blood flow measurements would be very fast (fractions of seconds to seconds). In other embodiments, image guidance for surgical procedures could be continuous for hours. In still other embodiments, additionally, timelapse imaging of biological processes, such as continuous imaging of aqueous flow changes before and after drug or surgical therapies may be minutes to hours.

In embodiments, the memory further comprises instructions, which, when executed by the processor, cause the processor to: instruct the optical system to manipulate the structure. In some cases, manipulating the structure comprises using the excitation source for non-incisional therapy. In other cases, the non-incisional therapy comprises one or more of: multi-photon-mediated damage, such as thermal damage, photo-disruption, photo cross-linking, blood vessel coagulation, ablating the structure.

In certain embodiments, manipulating the structure comprises using the excitation source to treat glaucoma. In some cases, using the excitation source to treat glaucoma comprises using the excitation source to reduce aqueous production of ocular tissue. In other cases, using the excitation source to reduce aqueous production of ocular tissue comprises damaging ciliary body, thermally damaging ciliary body or applying a photo disruption mediated process, in each case, to reduce aqueous production. In still other cases, using the excitation source to treat glaucoma comprises using the excitation source to increase outflow of aqueous humor. In embodiments, using the excitation source to increase outflow of aqueous humor comprises performing laser trabeculotomy through the non-transparent tissue of the eye. In certain cases, performing laser trabeculoplasty comprises performing laser trabeculoplasty directly through the non-transparent tissue of the eye.

In embodiments, manipulating the structure comprises using the excitation source to prevent or treat retinal breaks, such as retinal tears. In some cases, preventing or treating retinal tears comprises identifying and providing photocoagulation therapy. In embodiments, the retinal break or tear is a peripheral retinal break or tear.

In other embodiments, manipulating the structure comprises providing photocoagulation and thermal treatment to the structure, such as to ciliary body tumors. In still other embodiments, manipulating the structure comprises providing photocoagulation and thermal treatment to peripheral choroidal tumors. In certain embodiments, manipulating the structure comprises optically cross-linking the scleral tissue for prevention of myopia. In embodiments, manipulating the structure comprises visualizing and performing targeted alteration of extraocular muscle function. In other embodiments, manipulating the structure comprises visualizing and performing targeted thermal or photocoagulation therapy of orbital fat. In still other embodiments, manipulating the structure comprises visualizing and performing targeted alteration of palpebral tissues.

In instances, the processor and/or memory may be operably connected to the optical system and in some cases, the adaptor. In embodiments, the processor and/or memory are operably connected to certain aspects of the optical system. Such operable connection may take any convenient form such that data and/or control signals generated by the processor, the optical system and/or the adaptor may be transmitted therebetween by any convenient input/output technique, such as via a wired or wireless network connection, shared memory, a bus or similar communication protocol, such as an ethernet connection or a Universal Serial Bus (USB) connection, portable memory devices or the like.

Any convenient processor and memory may be used in embodiments of the subject systems. For example, any off the shelf, commercially available processor or memory such as those discussed in detail above, may be used. In particular, in embodiments, the processor may comprise a general purpose processor or a processor comprising a plurality of multi-core processors or parallel processing units, such as, for example, a graphics processing unit or other processor configured to support parallel processing operations, or combinations thereof. In instances, the processor and memory are operably connected to each other. Such operable connection may take any convenient form such that instructions and data may be obtained by the processor by any convenient input technique, such as via a wired or wireless network connection, shared memory, a bus or similar communication protocol.

### Exemplary Embodiments of Systems of the Present Invention:

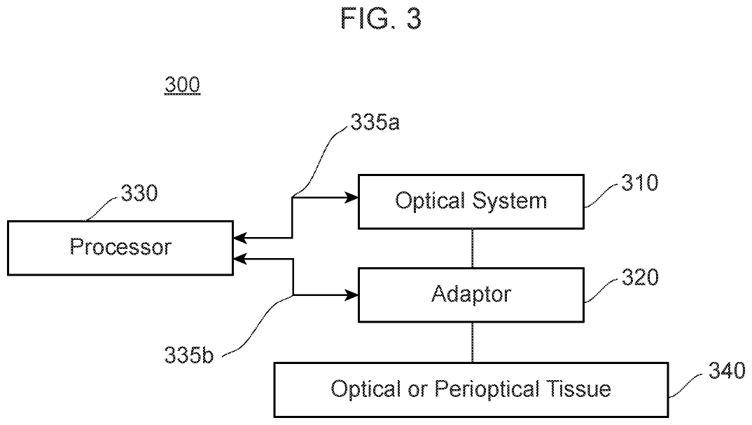

FIG. 3 illustrates a schematic view of a system 300 for imaging a structure through non-transparent ocular or periocular tissue according to an embodiment of the present invention. The embodiment of system 300 of the present invention depicted in FIG. 3 relates to a system for imaging any convenient structure accessible through non-transparent tissue, such as ocular or periocular tissue, according to the present techniques and such may vary. System 300 is an exemplary embodiment of the present invention provided for illustrative purposes, and aspects thereof may vary as desired in embodiments of the present invention.

As seen in FIG. 3, system 300 comprises optical system 310 that interfaces with each of processor 330 and adaptor 320. In embodiments, optical system 310 may comprise both an excitation source for stimulating light in a structure as well as a detector for detecting stimulated light originating from the imaged structure. In embodiments, adaptor 320 may be shaped so as to receive optical system 310; e.g., mechanically receive such as by holding or supporting optical system or aspects thereof. Either or both of adaptor 320 and optical system 310 may be capable of translating, rotating or otherwise articulating relative to optical or periocular tissue 340 (FIG. 3 specifies optical or periocular tissue for illustrative purposes only and the present disclosure is not limited as such). Optical system 310 is operably connected to processor 330 via operable connection 335a, which may take the form of a wired or wireless connection, such as, for example a universal serial bus (USB) connection or Bluetooth connection or the like. In system 300, processor 330 is operably connected to adaptor 320 via operably connection 335b, which may take the form of a wired or wireless connection, such as, for example a universal serial bus (USB) connection or Bluetooth connection or the like. Operable connections 335a, 335b may be configured to transmit data signals, such as data regarding light detected from the structure and/or control signals, such as signals instructing the optical system to transmit specified light energy over a specified area or volume or the like. In connection with system 300, non-transparent tissue comprises any convenient non-transparent ocular or periocular tissue and such may vary (and, as noted above, embodiments of systems of the present invention are not limited to ocular or periocular tissue and may include other tissues, such as, for example, dermatologic tissues accessed in connection with dermatologic procedures or cosmetic procedures or the like).

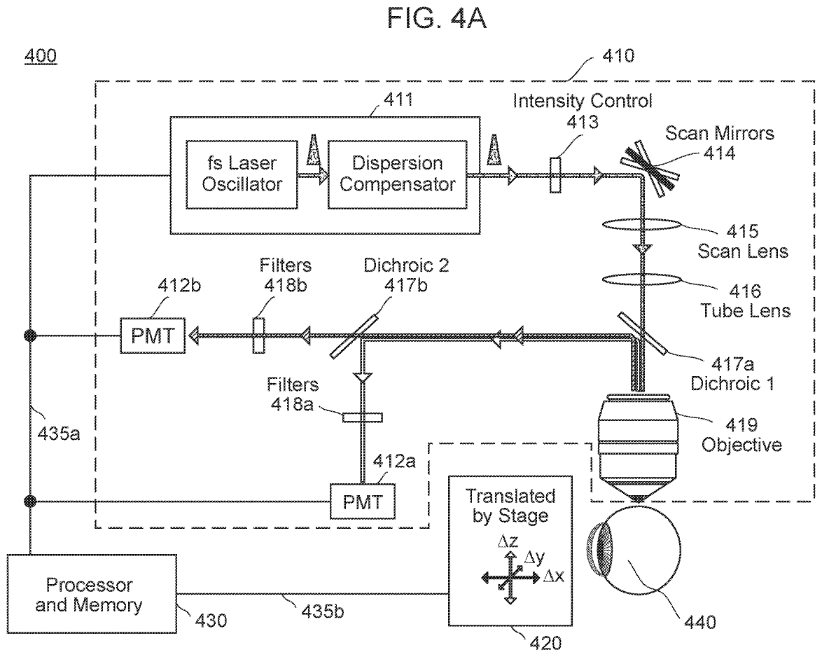

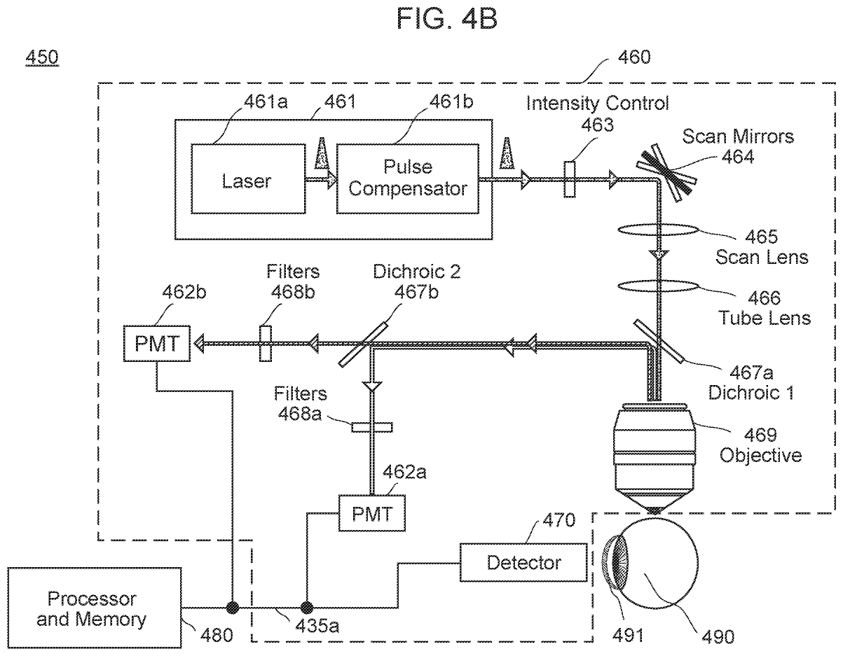

FIG. 4A illustrates an exemplary system 400 for imaging a structure through non-transparent tissue, such as ocular or periocular tissue, according to an embodiment of the present invention. The embodiment of system 400 of the present invention depicted in FIG. 4A relates to a system for imaging any convenient structure accessible through non-transparent tissue, such as ocular or periocular tissue, according to the present techniques, and such may vary. System 400 is an exemplary embodiment of the present invention provided for illustrative purposes, and aspects thereof may vary as desired in embodiments of the present invention. System 400 is employed through the sclera of non-transparent ocular tissue 490.

As seen in FIG. 4A, system 400 comprises optical system 410 that interfaces with each of processor 430 and adaptor 420. In embodiments, optical system 410 may comprise both an excitation source 411 for stimulating light in structure 440 as well as first detector 412a and second detector 412b for detecting stimulated light originating from the imaged structure. Optical system 410 further comprises optical elements used in conjunction with transmitting light energy to the structure through non-transparent tissue, such as ocular or periocular tissue: intensity control element 413; scan mirrors 414 configured to move so as to adjust the position or angle of light transmitted from excitation source 411; scan lens 415; and tube lens 416. In embodiments, the scan and tube lenses are static optics that are chosen and/or configured to help define the size of the imaging field. In embodiments, such scan/tube lenses may be different to optimize for different objective lenses. Such is particularly important when it is desired to scale up for human eyes and imaging as much of the eye as possible in a single scan. In embodiments, different scan/tube lenses may be employed for different setups or applications.

Optical system 410 further comprises the following optical elements used in conjunction with light emitted from the structure via multiphoton excitation: dichroic 1 417a; dichroic 2 417b; first filter 418a; and second filter 418b. Dichroic 1 417 a, dichroic 2 417b, first filter 418a and second filter 418b are configured such that light corresponding to certain wavelengths or ranges of wavelengths is reflected or allowed to pass. In embodiments, filters and/or dichroics are custom made by companies through a sputtered-oxide thin film technique. Wavelength specifications are highly variable across embodiments. Such are commercially available from Semrock or Chroma, for example.

Optical system 410 further comprises objective 419, i.e., objective lens. As seen in the placement of objective lens 419 within optical system 410, both light transmitted from excitation source 411 to an imaged structure present within ocular tissue 440, as well as light collected from the imaged structure present within ocular tissue 440 pass through objective lens 419. That is, the same objective lens 419 is used both for delivering light from excitation source 411 and for signal collection. By signal collection, it is meant collecting, or detecting, signal, i.e., emitted or stimulated light, generated at the focus of the laser, i.e., by a structure present within ocular tissue 440.

In embodiments, adaptor 420 may be configured so as to receive optical system 410; e.g., mechanically receive optical system 410 such as by holding or supporting optical system 410. Adaptor 420 interfaces with optical system 410 and ocular tissue 440 or periocular tissue or other tissue (not shown in FIG. 2B). Adaptor 420 is shown as capable of translating, rotating or otherwise articulating relative to optical or periocular tissue 440. As adaptor 420 translates, rotates or otherwise articulates relative to optical or periocular tissue 440, adaptor 420 may cause optical system 410 to correspondingly translate, rotate or otherwise articulate. Aspects of optical system 410 are operably connected to processor 430 via operable connection 435a, which may take the form of a wired or wireless connection, such as, for example a universal serial bus (USB) connection or Bluetooth connection or the like. In system 400, processor 430 is shown as operably connected to adaptor 420 via operable connection 435b, which may take the form of a wired or wireless connection, such as, for example a universal serial bus (USB) connection or Bluetooth connection or the like.

Operable connection 435a may be configured to transmit data signals, such as data regarding light detected from the structure and/or control signals, such as signals instructing the optical system to transmit specified light energy over a specified area or the like. In connection with system 400, non-transparent ocular tissue comprises a top surface of an eye.

FIG. 4B illustrates alternative exemplary system 450 for imaging a structure through non-transparent ocular or periocular tissue according to an embodiment of the present invention. System 450 illustrates additional optical components present in embodiments of systems of the invention. While system 450 is also employed through the sclera of non-transparent ocular tissue 490, system 450 further illustrates an alternative mechanism for sensing light emitted from the imaged structure, i.e., detecting or collecting light for imaging the structure, comprising detector 470 positioned directly over cornea 491 of ocular tissue 490.

The embodiment of system 450 of the present invention depicted in FIG. 4B relates to a system for imaging any convenient structure accessible through non-transparent ocular or periocular tissue according to the present techniques and such may vary. System 450 is an exemplary embodiment of the present invention provided for illustrative purposes, and aspects thereof may vary as desired in embodiments of the present invention.

As seen in FIG. 4B, system 450 comprises optical system 460 that interfaces with processor and memory 480 as well as an adaptor (not shown in FIG. 4B). In embodiments, optical system 460 may comprise both an excitation source 461 for stimulating light in a structure as well as first detector 462a and second detector 462b for detecting stimulated light originating from the imaged structure. In optical system 460, excitation source 461 comprises laser 461a as well as pulse compressor 461b for adjusting, e.g., shortening pulse widths of pulsed light energy generated by laser 461a. Optical system 460 further comprises additional optical elements used in conjunction with transmitting light energy to the structure through non-transparent ocular or periocular tissue: intensity control element 463; scan mirrors 464 capable of moving so as to adjust the position or angle of light transmitted from excitation source 461; i.e., adjust a focal point of light transmitted from excitation source 461; scan lens 465; and tube lens 466. Optical system 460 may comprise further additional optical elements used in conjunction with transmitting light energy to the structure through non-transparent ocular or periocular tissue: a laser scanner to move the laser focus to throughout the image field in any desired scan pattern or raster (not shown in FIG. 4B); a power attenuator (not shown in FIG. 4B); or adaptive optics for wavefront shaping (not shown in FIG. 4B). Optical system 460 further comprises optical elements used in conjunction with light emitted from the structure via multiphoton excitation: dichroic 1 467a; dichroic 2 467b; first filter 468a; and second filter 468b. Dichroic 1 467a, dichroic 2 467b, first filter 468a and second filter 468b are configured such that light corresponding to certain wavelengths or ranges of wavelengths is reflected or allowed to pass. The embodiments shown in FIGS. 4A-B comprise two PMTs. This is a viable setup. However, other embodiments may comprise additional PMTs to make this (for example) 4 PMTs (i.e., comprising a third and fourth PMT) or more. In such a case, there would be additional dichroics, which split the light between first and second PMTS and third and fourth PMTS, respectively

Optical system 460 may interface with ocular tissue 490 via an adaptor (not shown in FIG. 4B) configured so as to receive optical system 460; e.g., mechanically receive optical system 460 such as by holding or supporting optical system 460.

Optical system 460 is operably connected to processor and memory 480 via operable connection 435a, which may take the form of a wired or wireless connection, such as, for example a universal serial bus (USB) connection or Bluetooth connection or the like. Operable connection 435a may be configured to transmit data signals, such as data regarding light detected from the structure and/or control signals, such as signals instructing the optical system 460 to transmit specified light energy over a specified area or the like. In connection with system 450, non-transparent ocular tissue 490 comprises a top surface of an eye.

Optical system 460 further comprises detector 470 positioned directly over cornea 491 of ocular tissue 490. Detector 470 may be any convenient sensor and include, for example, a contact lens, a filter and a photomultiplier tube (PMT) or the like, positioned directly on cornea 491 of ocular tissue 490. Detector 470 may be positioned to collect signal light that was emitted deep within ocular tissue 490, i.e., rather than signal that is back-scattered and collected by objective lens 469. In general, detector 470 may be positioned at any location relative to ocular tissue 490 capable of transmitting light emitted from structure via non-transparent ocular tissue 490. Detector 470 may be used in conjunction with, e.g., concurrently with, first and second detectors 462a and 462b or may be used separately from first and second detectors 462a and 462b. In some cases, first and second detectors 462a and 462b or detector 470 may be better suited for collecting signal from a certain structure or collecting signal through a certain tissue, such as certain ocular or periocular tissue, or collecting signal characterized by certain characteristics, such as a specified range of wavelengths. In general, in embodiments, the more signal that is collected by optical system 460, i.e., by utilizing each of first and second detectors 462a and 462b as well as detector 470, the better the resulting image quality.

Pulse compressor 461b, or dispersion compensation, is achieved by using a set of prisms to correct for chromatic dispersion of the laser 461a light as it travels to the focus in the imaged structure, i.e., tissue. The goal of employing pulse compressor 461b, or dispersion compensation, is to have the pulse as narrow as possible at the focus in the imaged structure, i.e., tissue. This can achieve the highest efficiency of multiphoton excitation. Pulse compressors of interest may be present within the laser system, such as laser 461a, or in an optical setup located after (i.e., distal to) the laser 461a. Further information regarding pulse compression and dispersion compensation is presented in: APE Angewandte Physik & Elektronik GmbH, Dispersion Compensation & Pulse Compression for Microscopy, https://www.ape-berlin.de/en/dispersion-compensation-pulse-compression/ (last visited Oct. 25, 2022), the disclosure of which is incorporated herein by reference in its entirety.

Methods of interest for achieving intensity control include deploying a pair of polarizers rotated relative to each other or Pockel Cell or acousto-optic modulator (AOM). Embodiments of the former approach are relatively inexpensive, and embodiments of the latter two approaches are capable of being extremely fast and are further capable of varying the laser power within different locations essentially as fast as the laser can be scanned.

Embodiments of scan mirrors 464 in the XY axes can include galvanometric mirrors or resonant mirrors or a combination of the two. Galvanometric mirrors enable precise targeting of specific locations (i.e., for laser treatment). Resonant mirrors enable relatively faster imaging. Rapid scanning in the Z axis can be more challenging and is typically achieved by one or more of:
piezo moving the objective lens or deformable focusing lens or deformable mirror. Further information regarding deformable mirrors is presented in: K. N. Ito, K. Isobe & F. Osakada, Fast z-focus controlling and multiplexing strategies for multiplane two-photon imaging of neural dynamics, Neuroscience Research, Volume 179, June 2022, Pages 15-23, available at: https://www.sciencedirect.com/science/article/pii/S0168010222000827, the disclosure of which is incorporated herein by reference in its entirety.

### Utility

The subject methods, adaptors and systems find use in a variety of applications where it is desirable to image through non-transparent tissue, such as ocular or periocular tissue, i.e., image a structure through tissues and at resolutions that are inaccessible with current imaging techniques. In some embodiments, the methods, adaptors and systems described herein find use in clinical settings such as any clinical setting in which there is a need for imaging a structure through non-transparent tissue, such as ocular or periocular tissue, in particular imaging structures previously inaccessible due to the presence of the non-transparent tissue. In addition, the subject methods, adaptors and systems find use in treating conditions, such as glaucoma or the treatment and prevention of retinal detachment or the treatment of ciliary body tumors or peripheral choroidal tumors. Further, the subject methods, adaptors and systems find use in gene therapy applications, such as applying gene therapy techniques to structures present in ocular tissue or periocular tissue.

The subject methods, adaptors and systems may find use as a transscleral imaging tool for visualizing ocular structures with high resolution, including intraocular structures previously inaccessible by optical approaches including the ciliary body, peripheral retina and choroid. Embodiments of methods, adaptors and systems according to the present invention are capable of providing cellular resolution and functional imaging capabilities, exceeding the information obtainable by other transscleral imaging approaches such as ultrasonography and MRI.

The subject methods, adaptors and systems may find use as a non-incisional therapy tool for precise photocoagulation and/or tissue disruption of the ciliary body to controllably and safely reduce aqueous production of the eye for the treatment of glaucoma.

The subject methods, adaptors and systems may find use as a non-incisional therapy tool for performing laser trabeculotomy and/or trabeculoplasty to increase outflow of aqueous humor for the treatment of glaucoma.

The subject methods, adaptors and systems may find use as a non-incisional therapy tool for identifying and providing photocoagulation therapy to peripheral retinal tears for the treatment and prevention of retinal detachment.

The subject methods, adaptors and systems may find use as a non-incisional therapy tool for identifying and providing photocoagulation and thermal treatment to ciliary body tumors and peripheral choroidal tumors.

The subject methods, adaptors and systems may find use as a non-incisional therapy tool for photo-crosslinking of the sclera.

The subject methods, adaptors and systems may find use in connection with applying gene or cell or drug therapy treatments, in particular as applied to ocular or periocular tissues.

Embodiments of the invention may find use imaging and/or manipulating tissue in clinical or experimental settings, including, for example, in connection with imaging neuronal activity, imaging a corneal nerve, imaging aspects of a central nervus system, utilizing an invasive probe to image aspects of a central nervus system, thinning a region of a skull to image aspects of a central nervus system, visualizing nerve structures, utilizing imaged nerve structures to mitigate pain, utilizing an imaged structure to perform flow analysis, performing flow analysis substantially in real time, generating a velocity map of fluid flow within an imaged structure, diagnosing disease, diagnosing cancer, distinguishing between cancerous and non-cancerous tissues, distinguishing between cancerous and non-cancerous cells, preventing or treating retinal breaks, providing non-thermal treatment, providing non-thermal treatment to tumors, visualizing orbital fat, manipulating dermatologic tissue, imaging one or more of epidermis, dermis or hypodermis, manipulating a tear duct to facilitate fluid flow within the tear duct, mitigating ocular tissue redness, delivering therapy to an imaged structure, guiding positioning of an intraocular implant based on the imaged structure, predicting effective placement of intraocular implant based on the imaged structure, guiding positioning of an intraocular implant based on the imaged structure while implanting the intraocular implant, evaluating a position of an implanted intraocular implant based on the imaged structure, evaluating an effective lens position (ELP) of an implanted intraocular implant based on the imaged structure, detecting cancerous tissue, such as melanoma, providing cosmetic treatment, providing cosmetic dermatologic treatment, providing cosmetic surgery, treating scars, removing scars, treating acne scars, removing acne scars, treating skin discoloration, removing birthmarks, removing port-wine stains, treating skin discoloration disorders, removing tattoos, treating rosacea, providing controlled cutting of tissue, providing controlled cutting of dermatologic tissue, performing a skin biopsy procedure, disrupting cancerous tissue, disrupting one or more cancer cells, ablating cancerous tissue, ablating one or more cancer cells, ablating melanoma, ablating skin melanoma, removing hair, disrupting hair follicles, ablating hair follicle tissue, affecting tissue shape, affecting a shape of one or more fat deposits, reducing a volume of one or more fat deposits, reducing one or more subdural fat deposits, providing tissue sculpting, providing skin tightening, treating dry eye syndrome, mitigating eye redness, providing pain management or perforating tissue, for example.

The following is offered by way of illustration and not by way of limitation.

### Experimental

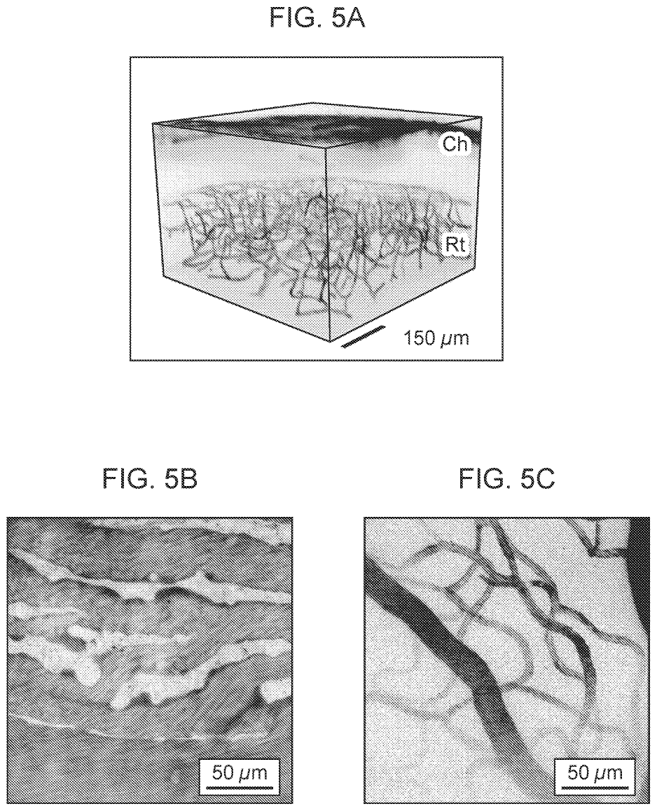

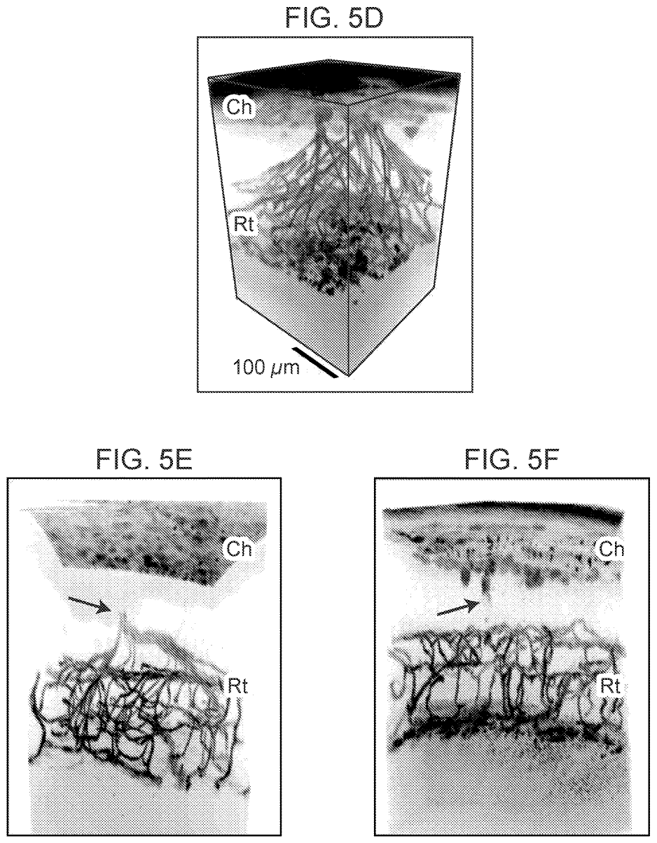

FIGS. 5A-F depict results utilizing embodiments of the present invention for 3PEF transscleral imaging to visualize the chorioretinal vasculature and chorioretinal anastomoses. FIG. 5A shows a three-dimensional projection of 3PEF data acquired through the intact sclera in whole, fixed pigmented mouse eye (Ch refers to choroid vessels, and Rt refers to retinal vessels). FIG. 5B shows intravital 2PEF imaging and projection of choroidal vasculature wherein the blood plasma is labeled with red-fluorescent dye. FIG. 5C shows intravital imaging and full-thickness projection of retinal vasculature directly underlying the choroid in FIG. 5B. FIG. 5D shows chorioretinal anastomoses in a mouse model of neovascular age-related macular degeneration (AMD). FIG. 5E shows developing chorioretinal anastomosis originating from the retinal vasculature. FIG. 5F shows developing chorioretinal anastomosis originating from choroidal vasculature. FIGS. 5A-F demonstrate an investigation into the developmental mechanisms and molecular identity of chorioretinal neovascularization and formation of anastomoses and further elucidate the role of artery-vein identity on subsets of disease that are resilient to existing therapies. FIGS. 5A-F illustrate that multiphoton microscopy, utilized in embodiments of the present invention, is a powerful technique for intravital imaging deep into tissues with subcellular resolution. Transpupillary two-photon excited fluorescence (2PEF) microscopy has been combined with dispersion compensation and adaptive optics (AO) correction to overcome corneal and lenticular aberration to provide elegant imaging of the retina and photoreceptors. Further details are provided in: Palczewska, G., et al., Noninvasive two-photon microscopy imaging of mouse retina and retinal pigment epithelium through the pupil of the eye. Nat Med, 2014. 20(7): p. 785-9 as well as Palczewska, G., T. S. Kern, and K. Palczewski, Noninvasive Two-Photon Microscopy Imaging of Mouse Retina and Retinal Pigment Epithelium. Methods Mol Biol, 2019. 1834: p. 333-343, the disclosures of which are incorporated herein in their entireties. However, to the inventors'knowledge, current 2PEF technologies have been unable to image into the choroid due to intrinsic optical constraints imposed by transpupillary imaging and the high absorptivity of the RPE and choroid. As demonstrated in FIGS. 5A-F, embodiments of the present invention can successfully achieve transscleral imaging of chorioretinal vasculature in intact C57BL/6 mouse eyes. Embodiments of the present invention enable chorioretinal imaging in albino and pigmented eyes using conventional 2PEF, extended-infrared 2PEF, 3PEF, 2HG, 3HG and AO correction. Imaging using embodiments of the present invention is facilitated in part because transscleral imaging is not subject to the same focusing constraints as transpupillary imaging, and therefore embodiments of the present invention utilizing transscleral multiphoton microscopy are capable of improved spatial resolution for multiphoton imaging of the retina (most notably axial resolution). To the inventors'knowledge, quantification of blood flow in the choroid has not previously been achieved at the micro-vessel level. Further, combining transscleral imaging with a robust analysis method can quantify blood flow in choroid down to the micron-scale. Further details are provided in: Kim, T. N., et al., Line-scanning particle image velocimetry: an optical approach for quantifying a wide range of blood flow speeds in live animals. PLoS One, 2012. 7(6): p. e38590, the disclosure of which is incorporated herein in its entirety.

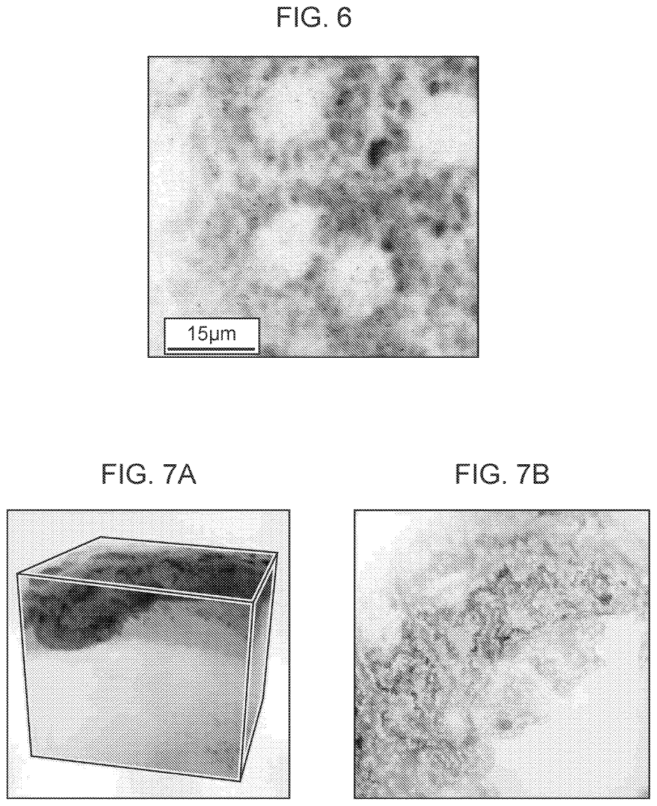

FIG. 6 depicts results of utilizing an embodiment of the present invention for 3PEF transscleral imaging to visualize retinal pigmented epithelium. Specifically, FIG. 6 shows results of transscleral 3PEF imaging of the RPE in pigmented mouse eye resolving nuclei, melanosome granules and cell boundaries.

FIGS. 7A-B depict results of utilizing embodiments of the present invention for 3PEF transscleral imaging of the ciliary body. FIG. 7A shows a three-dimensional projection of the ciliary body from image data acquired through the intact sclera. FIG. 7A is a three-dimensional projection of approximately 45 degrees of the ciliary body in living mouse eye. FIG. 7B shows one cross-sectional slice from three-dimensional data in FIG. 7A demonstrating the processes of the ciliary body with cellular resolution and distinction of the epithelium responsible for producing the aqueous humor of the eye.

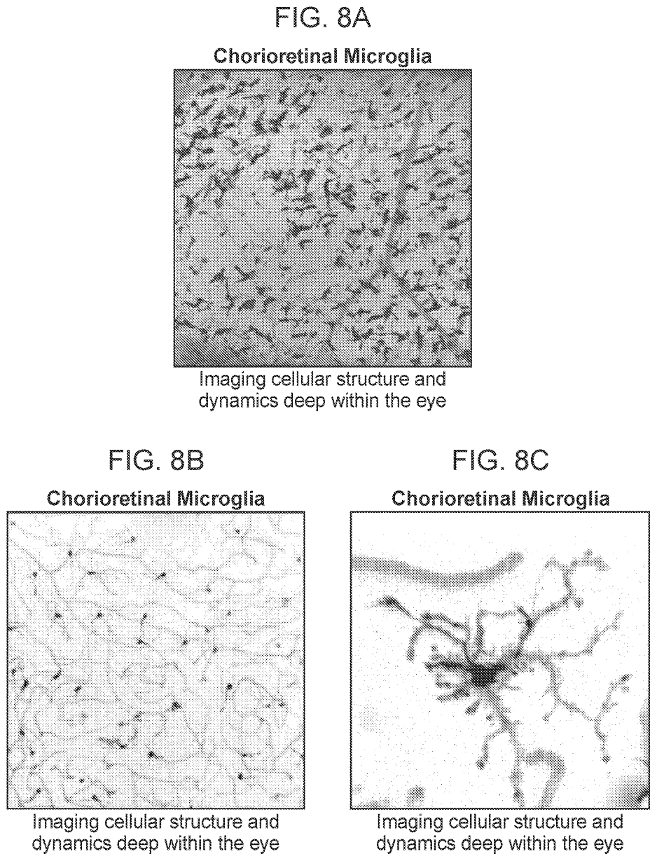

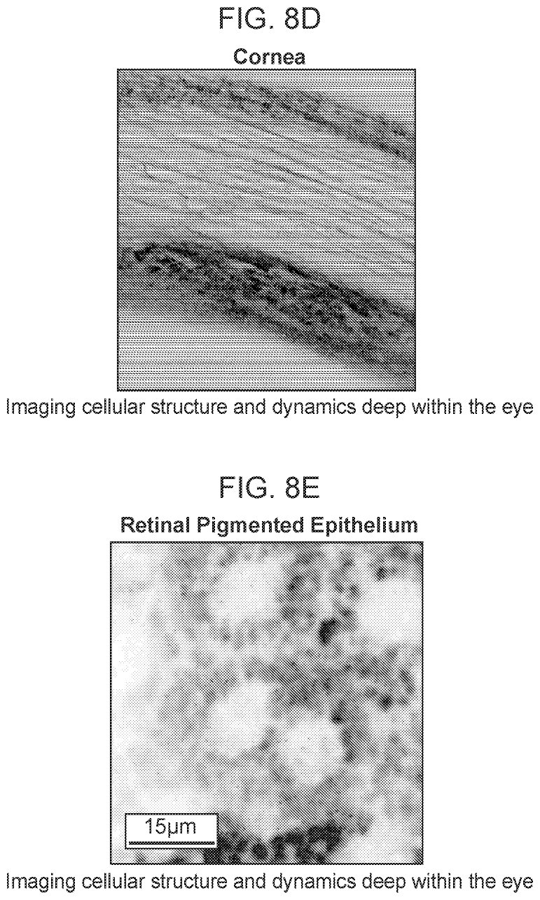

FIGS. 8A-E depict results of utilizing embodiments of the present invention for imaging intravital cellular imaging in the living eye. In particular, FIGS. 8A-E depict imaging cellular structure and dynamics deep within the eye. FIGS. 8A-C show results of imaging chorioretinal microglia, in particular, green fluorescent protein (GFP) expression in cells from a transgenic mouse. FIG. 8D shows results of imaging a cornea, in particular, second harmonic generation (SHG) imaging of a cornea. FIG. 8E shows results of imaging retinal pigmented epithelium (RPE).

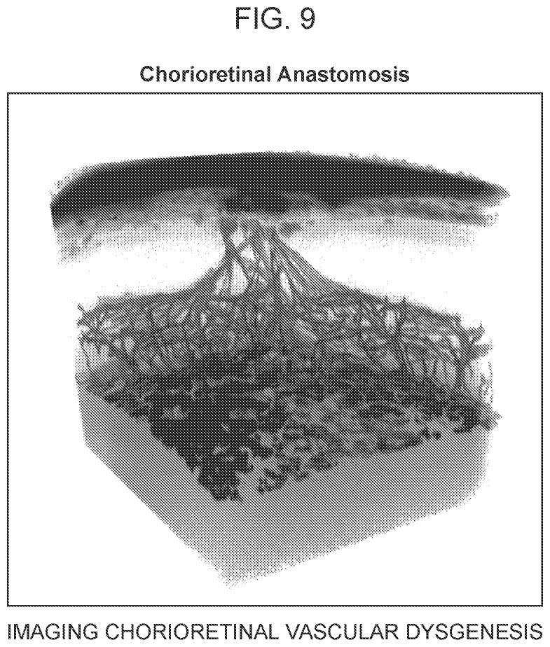

FIG. 9 depicts results of utilizing an embodiment of the present invention for imaging chorioretinal vascular dynamics. Specifically, FIG. 9 shows imaging results of chorioretinal anastomosis. The structure and imaging results of FIG. 9 demonstrate how spontaneous chorioretinal angiogenesis develops in a model of neovascular AMD, and further how chorioretinal anastomoses forms and can mature into large vascular connections.

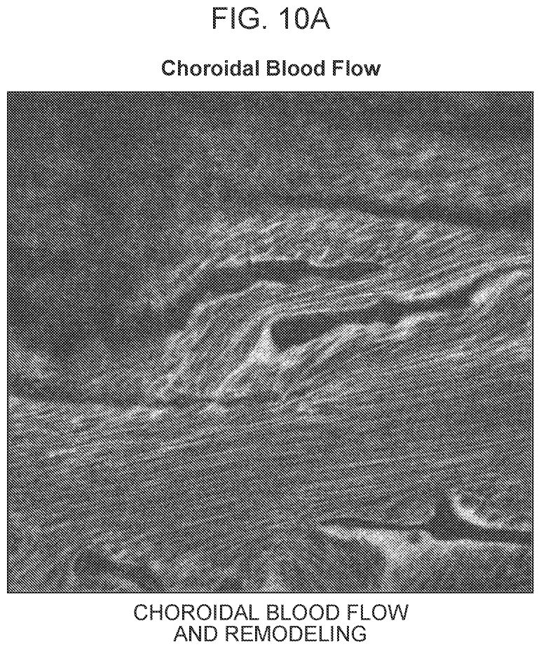

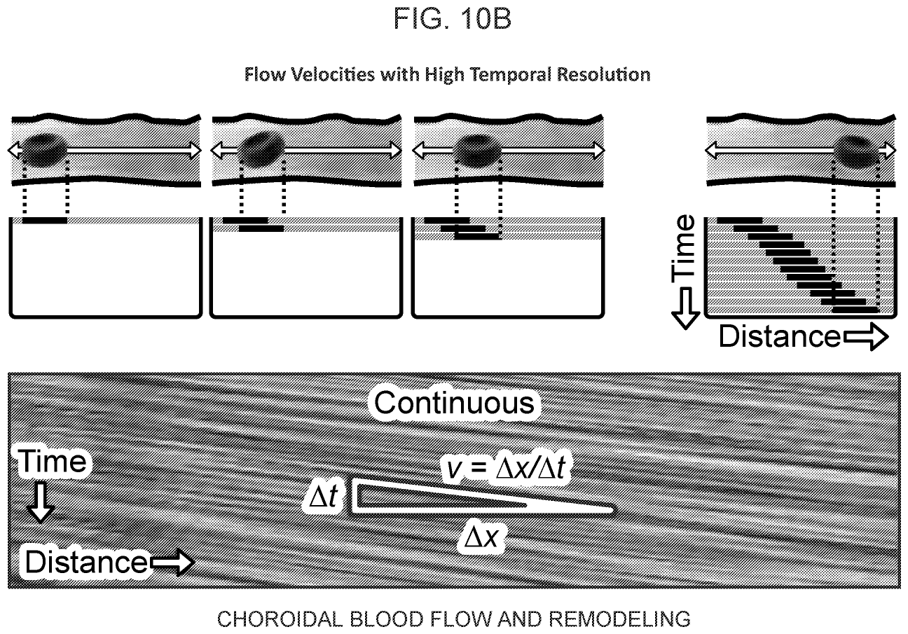

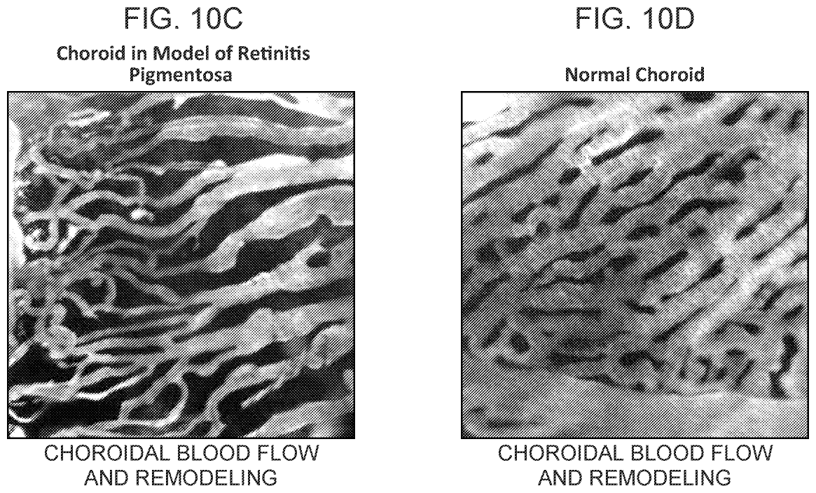

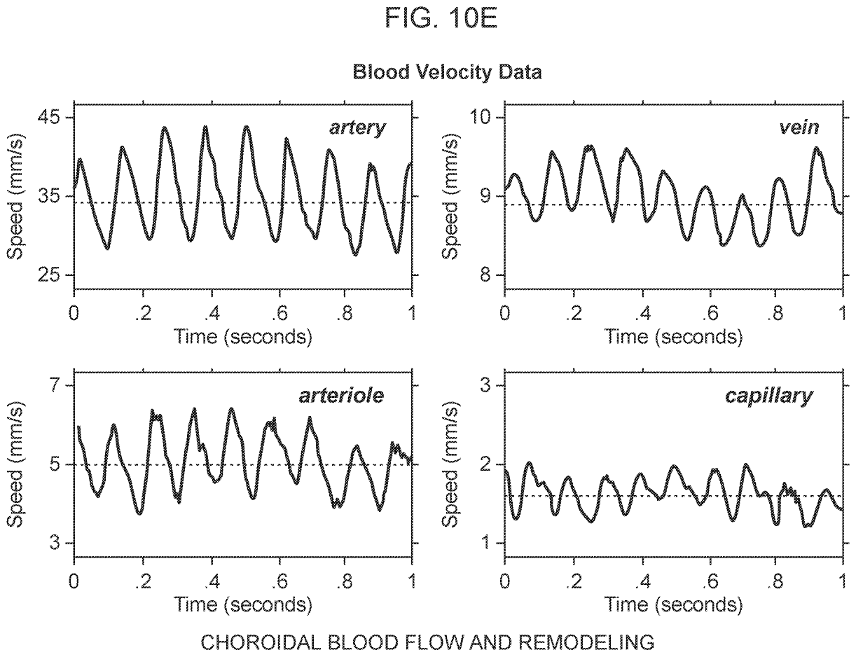

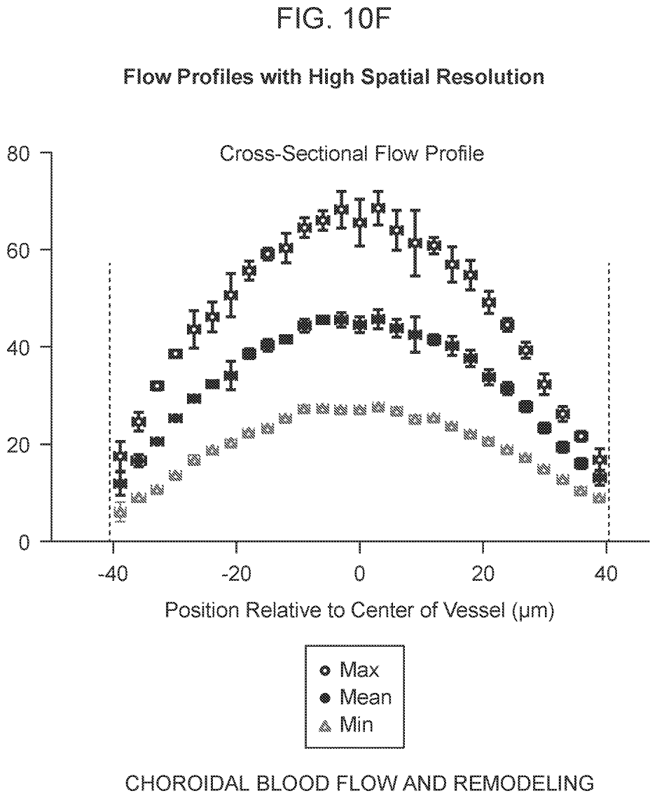

FIGS. 10A-F depict results of utilizing embodiments of the present invention for imaging choroidal blood flow and remodeling. FIG. 10A shows results of imaging choroidal blood flow. FIG. 10B shows results of imaging flow velocities with high temporal resolution as well as analytics applied to such imaging results. FIG. 10C shows results of imaging choroid in a model of retinitis pigmentosa. FIG. 10D shows results of imaging a normal choroid. FIG. 10E shows blood velocity data in different vascular tissues obtained utilizing imaging data. FIG. 10F shows flow profiles of fluid within a vessel with high spatial resolution obtained utilizing imaging data.

As described herein, embodiments of the present invention comprise utilizing multi-core processors, such as, for example, graphics processing units, in connection with conducing flow analysis, such as flow analysis of fluid through the choroid resulting in, for example, high-spatial resolution analyses such as that illustrated in FIG. 10F. Such embodiments that utilize multi-core processors are capable of providing real time flow analysis, whereas techniques that a single processing unit may not be capable of providing real time results.

For example, an embodiment of the present invention utilizing a single processing unit, e.g., a single CPU, used in connection with 5.0 seconds of acquisition at 100 KHz sampling rate results in a total analysis time of 3,418.41 s with an analyze-to-acquisition ratio of 683.74. This embodiment corresponds to a real time acquisition limit of 146 Hz. Such results are not viable for use in real time flow analysis. In embodiments, analyze-to-acquire refers to the ratio of wall-clock time (e.g., seconds) it takes to analyze a given amount of data, to the wall-clock time (e.g., seconds) it takes to acquire that amount of data. In instances, this metric is useful because: (1) it is independent of timescale (e.g., wall-clock time could be milliseconds or minutes) and (2) a real-time analyze-to-acquire ratio is intuitive, at 1 or less. This 683.74 means that however long you spend acquiring data, you need to spend 683.74 times as long as you spend acquiring it to analyze it.

In contrast to the above, for example, an embodiment of the present invention utilizing a graphics processing unit with a plurality of multi-core processors, used in connection with 5.0 seconds of acquisition at 100 KHz sampling rate results in a total analysis time of 1.76 s with an analyze-to-acquisition ratio of 0.35. This embodiment corresponds to a real time acquisition limit of 283,959 Hz. Such results, in contrast to the single CPU embodiment, are viable for use in real time flow analysis. In connection with embodiments described and reported on herein, a GPU comprises an NVIDIA Amphere or Ada Lovelace Architecture Card, and, in general, other commercially available GPUs may be employed, such as, for example, GPUs commercially available from AMD.

Embodiments of the present invention used to capture images seen in FIGS. 5-9 comprise one or more of: a Bergamo Series II Multiphoton Microscope System used with Spectra-Physics InSight X3 Tuneable Femtosecond laser; a high numerical aperture objective lens, N25X-APO-MP made by Nikon and commercially available from ThorLabs; detectors used comprise photomultiplier tubes (PMT2100 by ThorLabs); fluorophores used to generate such images include but are not limited to: Dextran, Texas Red by Invitrogen, TRITC-Dextran (Tetramethylrhodamine isothiocyanate-dextran) by Sigma Aldrich, FITC-Dextran (Fluorescein isothiocyanate-dextran), eGFP (enhanced Green Fluorescent Protein) and tdTomato (a bright red fluorescent protein). Embodiments of the present invention used in connection with the three-photon image of the corneal collagen fibers and retinal pigmented epithelium (FIGS. 8D-E) comprise a similar microscope, but with appropriate lens/mirror coatings to transmit 1,700 nm light. The laser used for these two images (FIGS. 8D-E) comprises the Coherent Monaco, as such is described herein. The signal for the cornea collagen is second harmonic generation and the RPE is third harmonic generation (both are intrinsic signal from the tissue without use of exogenous fluorophore).

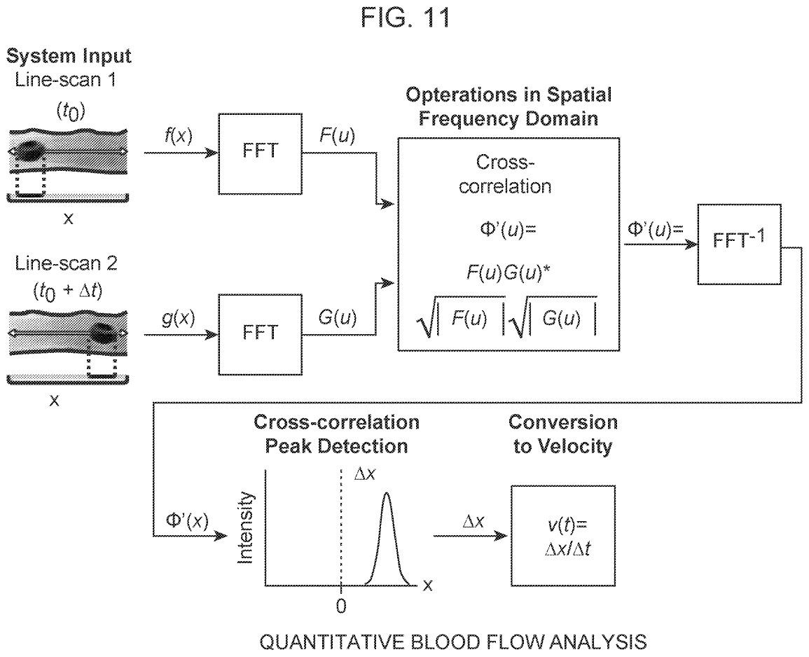

FIG. 11 depicts an exemplary quantitative analytical technique for blood flow analysis based on imaging data, according to an embodiment of the present invention. Embodiments of such modeling technique facilitate quantitative blood flow analysis within tissues, for example, blood flow analysis of choroid when combined with transscleral imaging, i.e., multiphoton imaging techniques described herein.

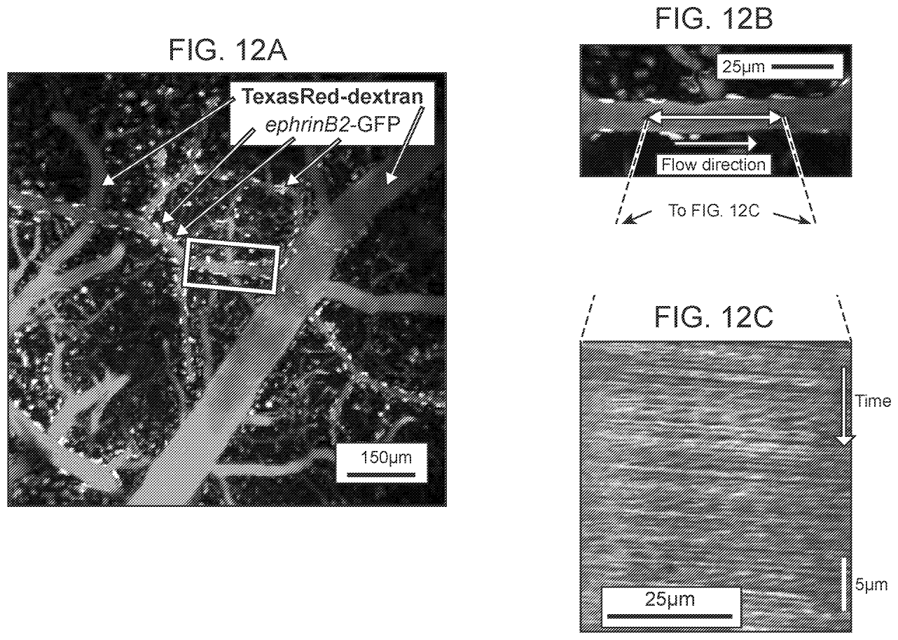

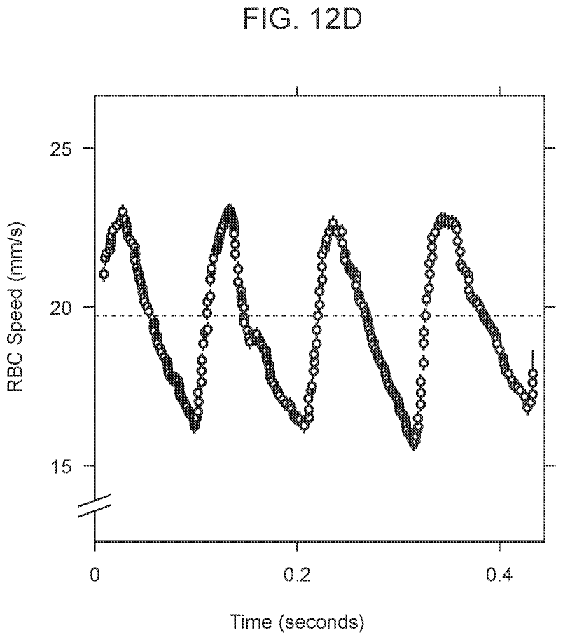

FIGS. 12A-D depict hemodynamic analysis with line-scanning particle image velocimetry according to embodiments of the present invention. FIG. 12A shows 2PEF imaging of vasculature in Ephrin-B2^{+/H2B-eGFP} wherein nuclear GFP distinguishes arterial from venous endothelial cells. FIG. 12B shows 2PEF image of an arteriole from the white box in FIG. 12A. FIG. 12C shows line-scan data where each sequential line-scan appears beneath the one before, forming a space-time image wherein dark streaks represent a moving RBC. FIG. 12D shows analysis of vessel center velocity with heart beats. As described above, FIG. 10F also shows cross-sectional analysis of flow profiles across the vessel lumen during cardiac cycle (max, mean, min).

The choroid is a unique and poorly understood vascular bed with variable flow and substantial contractility from non-vascular smooth muscle. Further information is set forth in: Poukens, V., B. J. Glasgow, and J. L. Demer, Nonvascular contractile cells in sclera and choroid of humans and monkeys. Invest Ophthalmol Vis Sci, 1998. 39(10): p. 1765-74, as well as May, C.A., Nonvascular smooth muscle alpha-actin positive cells in the choroid of higher primates. Curr Eye Res, 2003. 27(1): p. 1-6, the disclosures of each of which are incorporated herein in their entireties. Using intravital transscleral multiphoton microscopy according to embodiments of the present invention, high-resolution imaging of the choroid can be achieved (FIG. 12B) and blood flow data at the micro vessel scale can be gathered. In embodiments, transscleral multiphoton microscopy, combined with line-scanning particle image velocimetry (LS-PIV), enables quantification of choroidal blood flow with high spatiotemporal resolution. LS-PIV is a robust analytical method to analyze blood flow data generated by multiphoton imaging (as seen in, for example, FIGS. 12A-D and 10F), which finds use in research and clinical contexts with a diverse range of systems, including, for example, hindlimb and spinal cord. Further information regarding such techniques is set forth in: Kim, T. N., et al., Line-scanning particle image velocimetry: an optical approach for quantifying a wide range of blood flow speeds in live animals. PLoS One, 2012. 7(6): p. e38590, as well as Lasch, M., et al., Estimating hemodynamic shear stress in murine peripheral collateral arteries by two-photon line scanning. Mol Cell Biochem, 2019. 453(1-2): p. 41-51, as well as Chen, C., et al., An In Vivo Duo-color Method for Imaging Vascular Dynamics Following Contusive Spinal Cord Injury. J Vis Exp, 2017(130), the disclosures of each of which are incorporated herein in their entireties.

Embodiments of the present invention employ LS-PIV to analyze blood flow along vessel segments of the choroid and choriocapillaris including capillaries, venules, veins, arterioles, arteries. Because the morphology of choroidal vasculature is unique, hemodynamic analysis may be performed in a subset of Ephrin-B2^{+/H2B-eGFP} mice, wherein nuclear-GFP is expressed under the Ephrin-B2 promoter, allowing vessels to be distinguished with arterial molecular identity (as seen in, for example, FIG. 12A). Further information is set forth in: Davy, A., J. O. Bush, and P. Soriano, Inhibition of gap junction communication at ectopic Eph/ephrin boundaries underlies craniofrontonasal syndrome. PLoS Biol, 2006. 4(10): p. e315, as well as Murphy, P.A., et al., Constitutively active Notch4 receptor elicits brain arteriovenous malformations through enlargement of capillary-like vessels. Proc Natl Acad Sci USA, 2014. 111(50): p. 18007-12, as well as Murphy, P. A., et al., Notch4 normalization reduces blood vessel size in arteriovenous malformations. Sci Transl Med, 2012. 4(117): p. 117ra8, the disclosures of each of which are incorporated herein in their entireties.

An exemplary embodiment of a system according to the present invention has been constructed that comprises an optimized microscope for highly efficient collection and detection of fluorescence and which is capable of excitation wavelengths up to 1.7 μm. The primary excitation source is an advanced solid-state ytterbium-doped fiber laser with a usable power band between 350 nm to 5.0 μm, such as between 680 nm to 1.3 μm, and dispersion compensation to further improve excitation efficiency. The second excitation source is a fixed 1,045 nm laser with high power exceeding 3.5 W for additional imaging or photo disruption applications. By photo disruption, it is meant vaporization of tissue achieved through, e.g., higher order processes such as three, four, or five photon mechanisms. A third 1.7 μm excitation source comprises Raman shifting of 1.5 μm light in a large mode area photonic crystal rod for excitation wavelengths between 1.3 and 3.6 μm. The microscope has the ability to articulate around a subject, e.g., an animal model, such as a mouse or the like, at an imaging plane not parallel to the floor which maximizes the regions of the subject's eye that can be imaged in single sessions. The system has a multi-modal laser scanner that can switch between resonant imaging or patterned point-scanning, which are necessary for studying fast cellular dynamics (e.g., neuronal activity with calcium indicators) or measuring hemodynamics, respectively.

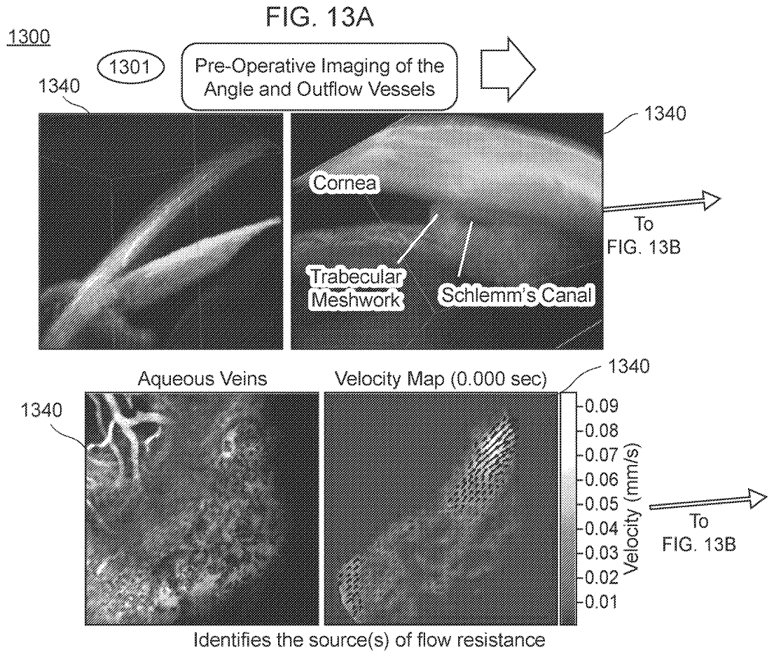

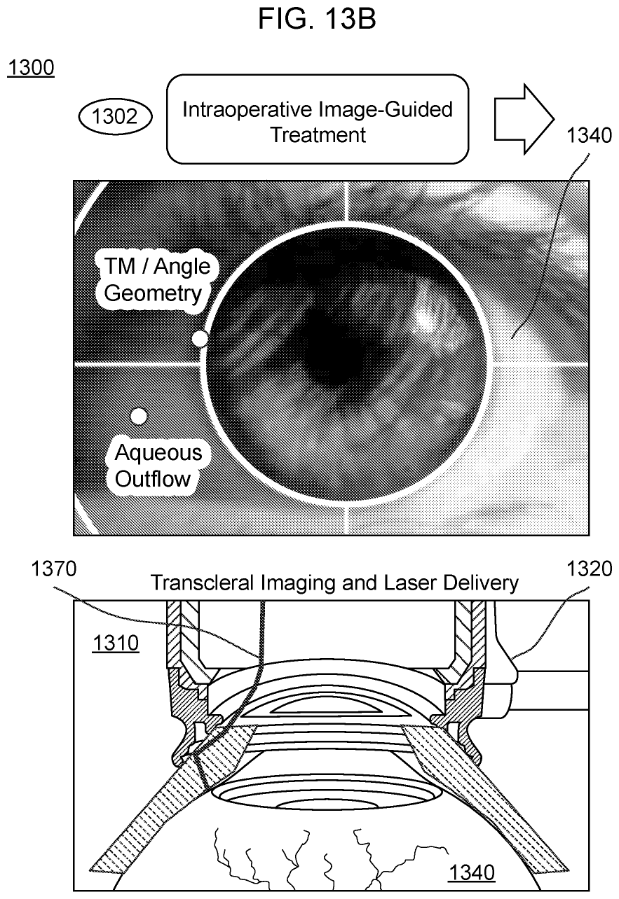

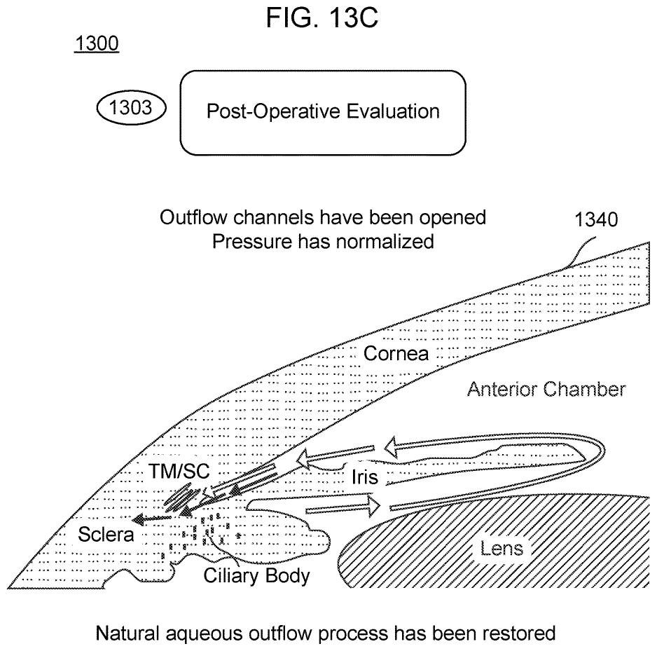

FIGS. 13A-C present an outline of an ophthalmic imaging and therapeutic application of embodiments of the present invention. In particular, FIGS. 13A-C depict steps of flow diagram 1300 for utilizing embodiments for imaging and optical manipulation for glaucoma analysis and treatment, according to an embodiment of the present invention.

Glaucoma is a progressive optic neuropathy and the leading cause of irreversible blindness worldwide. See Quigley, H. A. and A. T. Broman, The number of people with glaucoma worldwide in 2010 and 2020. Br J Ophthalmol, 2006. 90(3): p. 262-7, incorporated herein by reference. The only approach available through existing techniques to slow disease progression is reduction of intraocular pressure (IOP), which is regulated by aqueous humor production from the ciliary body and drainage through outflow pathways. See Weinreb, R. N., T. Aung, and F. A. Medeiros, The pathophysiology and treatment of glaucoma: a review. JAMA, 2014. 311(18): p. 1901-11, incorporated herein by reference. Transscleral laser-induced cyclodestruction is a class of non-incisional intervention wherein the ciliary body is damaged to inhibit aqueous humor production. In existing techniques, this approach has been reserved for late therapy due to potential complications including pain, vision loss and phthisis. See Dastiridou, A. I., et al., Cyclodestructive Procedures in Glaucoma: A Review of Current and Emerging Options. Adv Ther, 2018. 35(12): p. 2103-2127; Quigley, H. A., Histological and physiological studies of cyclocryotherapy in primate and human eyes. Am J Ophthalmol, 1976. 82(5): p. 722-32, incorporated herein by reference. Recent improvements in delivery of laser energy to the ciliary body have increased safety but at the expense of sustained IOP-lowering response. See Tan, A. M., et al., Micropulse transscleral diode laser cyclophotocoagulation in the treatment of refractory glaucoma. Clin Exp Ophthalmol, 2010. 38(3): p. 266-72; Garcia, G. A., et al., Micropulse Transscleral Diode Laser Cyclophotocoagulation in Refractory Glaucoma: Short-Term Efficacy, Safety, and Impact of Surgical History on Outcomes. Ophthalmol Glaucoma, 2019. 2(6): p. 402-412; Varikuti, V. N. V., et al., Outcomes of Micropulse Transscleral Cyclophotocoagulation in Eyes With Good Central Vision. J Glaucoma, 2019. 28(10): p. 901-905; Zaarour, K., et al., Outcomes of Micropulse Transscleral Cyclophotocoagulation in Uncontrolled Glaucoma Patients. J Glaucoma, 2019. 28(3): p. 270-275, incorporated herein by reference. The mechanism of this treatment failure is unclear, though prior work has correlated regeneration of the ciliary body to rise in IOP.

Embodiments of the present invention provide a novel therapy for safe, highly effective, and lasting treatment of the ciliary body in patients with glaucoma. Certain embodiments, referred to as Transscleral Multiphoton Image-guided Laser Therapy (TMILT), allow detailed imaging of the ciliary body with cellular resolution and equally precise laser treatment. Embodiments comprising TMILT do not require contrast or photosensitizing agents and can utilize intrinsic signal from tissues including multiphoton-excited autofluorescence and second-and third-harmonic generation. Embodiments comprising TMILT are capable of precisely targeting the ciliary body while avoiding damage to neighboring tissues to produce a sustained IOP lowering response. Such embodiments address a tremendous need to reduce surgical burden for glaucoma patients with treatment that is less invasive, safe, effective, and sustained.

For example, embodiments of the present invention induce precise multiphoton-mediated thermal damage to 180, 270, or 360 degrees of the ciliary body while containing visible laser-mediated damage to the ciliary body and avoiding adjacent structure. Other embodiments comprise intravital imaging to observe cellular dynamics contributing to ciliary body regeneration over serial timepoints in the same eye.

As described herein, multiphoton microscopy is capable of intravital imaging deep into tissues with subcellular resolution and is a powerful tool for studying cellular biology in living systems. See Helmchen, F. and W. Denk, Deep tissue two-photon microscopy. Nat Methods, 2005. 2(12): p. 932-40; Zipfel, W. R., R. M. Williams, and W. W. Webb, Nonlinear magic: multiphoton microscopy in the biosciences. Nat Biotechnol, 2003. 21(11): p. 1369-77, incorporated herein by reference. The depth of multiphoton imaging is intrinsically limited to ~5× the attenuation distance of an excitation wavelength within a tissue, and imaging through highly scattering tissues such as bone or sclera is a challenge. Embodiments of the present invention use next generation two-photon excitation (2PE), three-photon excitation (3PE), and adaptive optical correction to pass directly through the sclera to visualize underlying structure with subcellular resolution. Such embodiments also allow precise imaging and targeting of tissues for multiphoton-mediated damage, i.e., manipulation of a structure.

Further embodiments employ such imaging techniques in connection with Multiphoton-excited Aqueous Flowmetry (MAF) which relies on timelapse image data of the aqueous humor outflow (AHO) pathway and combine this with computational analysis, as described herein, to generate flow velocimetry maps. Still further embodiments comprise using transscleral multiphoton imaging to characterize the conventional AHO pathway with high-resolution and in three-dimensions by injecting fluorescein into the anterior chamber and mapping the outflow pathway including the anterior chamber, trabecular meshwork, Schlemm's Canal, collector channels, aqueous veins, and episcleral veins to create an atlas of detailed AHO anatomy and serve as a guide for subsequent applications, e.g., clinical or experimental uses. See Huang, A. S., et al., Aqueous Angiography: Aqueous Humor Outflow Imaging in Live Human Subjects. Ophthalmology, 2017. 124(8): p. 1249-1251; Saraswathy, S., et al., Aqueous Angiography: Real-Time and Physiologic Aqueous Humor Outflow Imaging. PLoS One, 2016. 11(1): p. e0147176; Helmchen, F. and W. Denk, Deep tissue two-photon microscopy. Nat Methods, 2005. 2(12): p. 932-40; Zipfel, W. R., R. M. Williams, and W. W. Webb, Nonlinear magic: multiphoton microscopy in the biosciences. Nat Biotechnol, 2003. 21(11): p. 1369-77; Wang, T., et al., Three-photon imaging of mouse brain structure and function through the intact skull. Nat Methods, 2018. 15(10): p. 789-792; Ouzounov, D. G., et al., In vivo three-photon imaging of activity of GCaMP6-labeled neurons deep in intact mouse brain. Nat Methods, 2017. 14(4): p. 388-390; Yildirim, M., et al., Functional imaging of visual cortical layers and subplate in awake mice with optimized three-photon microscopy. Nat Commun, 2019. 10(1): p. 177; Masihzadeh, O., et al., Third harmonic generation microscopy of a mouse retina. Mol Vis, 2015. 21: p. 538-47; Kim, T. N., et al., Line-scanning particle image velocimetry: an optical approach for quantifying a wide range of blood flow speeds in live animals. PLoS One, 2012. 7(6): p. e38590; Kazemi, A., et al., Effect of Timolol on Aqueous Humor Outflow Facility in Healthy Human Eyes. Am J Ophthalmol, 2019. 202: p. 126-132; Larsson, L. I., Aqueous humor flow in normal human eyes treated with brimonidine and timolol, alone and in combination. Arch Ophthalmol, 2001. 119(4): p. 492-5, incorporated herein by reference. See also H. Yamashita, M. S., Proof that the Ciliary Epithelium can Regenerate. Exp. Eye Res., 1978. 27: p. 199-213; Kuwahara, A., et al., Generation of a ciliary margin-like stem cell niche from self-organizing human retinal tissue. Nat Commun, 2015. 6: p. 6286; Cicero, S. A., et al., Cells previously identified as retinal stem cells are pigmented ciliary epithelial cells. Proc Natl Acad Sci U S A, 2009. 106(16): p. 6685-90; Del Debbio, C. B., et al., Rho GTPases control ciliary epithelium cells proliferation and progenitor profile induction in vivo. Invest Ophthalmol Vis Sci, 2014. 55(4): p. 2631-41; Gualdoni, S., et al., Adult ciliary epithelial cells, previously identified as retinal stem cells with potential for retinal repair, fail to differentiate into new rod photoreceptors. Stem Cells, 2010. 28(6): p. 1048-59; Barker, N., et al., Lgr5(+ve) stem/progenitor cells contribute to nephron formation during kidney development. Cell Rep, 2012. 2(3): p. 540-52; Humphreys, B. D. and D. P. DiRocco, Lineage-tracing methods and the kidney. Kidney Int, 2014. 86(3): p. 481-8; Park, M., et al., Peripheral (not central) corneal epithelia contribute to the closure of an annular debridement injury. Proc Natl Acad Sci U S A, 2019. 116(52): p. 26633-26643; Ventura, A., et al., Restoration of p53 function leads to tumour regression in vivo. Nature, 2007. 445(7128): p. 661-5; Moussa, K., et al., Histologic Changes Following Continuous Wave and Micropulse Transscleral Cyclophotocoagulation: A Randomized Comparative Study. Transl Vis Sci Technol, 2020. 9(5): p. 22, incorporated herein by reference.

In step 1301 of FIG. 13A, pre-operative imaging of the angle and outflow vessels of optical tissue are obtained utilizing imaging techniques according to the present invention. Such imaging in step 1301 identifies ocular and/or periocular anatomy within non-transparent tissue 1340 relevant to glaucoma such as the cornea, trabecular meshwork, Schlemm's canal and, in particular with respect to causing glaucoma symptoms, such as aqueous veins, as well as analysis of such relevant analytical structures, such as determinations of fluid flow velocity, which, together, identify one or more sources of flow resistance within the relevant anatomical structures and which may relate to conditions causing underlying glaucoma and symptoms thereof. The image showing aqueous veins in FIG. 13A is the result of transscleral 2PEF image of 2D blood flow data in an episcleral vein. Such imaging is analyzed to generate a two-dimensional blood flow analysis showing mean velocity map of RBC displacement in the blood flow.

From step 1301 in FIG. 13A, flow diagram 1300 next moves to step 1302 in FIG. 13B.

In step 1302 in FIG. 13B, intraoperative image-guided treatment is applied to the relevant anatomical structures visualized and analyzed in step 1301 present within non-transparent tissue 1340. Image-guided treatment comprises applying an embodiment of the present invention with optical system 1310 with element 1370 used to apply aspects of treatment (i.e., manipulation of tissue) to non-transparent ocular tissue 1340. In some cases, element 1370 is cable configured to route light energy, such as, for example, fiber optic cable. In such cases, element 1370 may be configured to route treatment light to non-transparent ocular tissue 1340, for example, in cases where imaging is used to guide application of treatment. In other cases, element 1370 may comprise other aspects of applying a treatment, such as a needle for providing an agent to non-transparent ocular tissue 1340 (e.g., in connection with providing stem cell therapy to non-transparent ocular tissue 1340), for example, in cases where imaging is used to guide needle position and placement of an injection. (While in many embodiments, treatment light is routed through the same optics as the imaging light, in other embodiments, such as, for example, embodiments configured for forms of cyclophotocoagulation, treatment light could be delivered separately by, for example, optical fiber(s) to specific locations of the eye, such as shown with respect to element 1370.) (In still other embodiments, element 1370 is configured to provide suction between adaptor 1320 and ocular tissue 1340.) Optical system 1310 is used to image as well as manipulate the anatomical structures identified in step 1301, including, for example, using optical system 1310 in connection with performing transscleral laser trabeculotomy and/or trabeculoplasty and/or sclerotomy to increase outflow of aqueous humor through, for example, conventional drainage pathways in connection with treatment of the underlying glaucoma. Element 1370 is routed through adaptor 1320 such that element 1370 can be positioned over non-transparent ocular tissue 1340.

From step 1302 in FIG. 13b, flow diagram 1300 next moves to step 1303 in FIG. 13C.

Step 1303 in FIG. 13C entails post-operative evaluation of the imaging and manipulation (e.g., laser trabeculotomy or trabeculoplasty) applied in step 1302 of FIG. 13B. In FIG. 13C, outflow channels within ocular tissue 1340 have been opened or expanded such that natural aqueous outflow process within ocular tissue 1340 is restored. Restoring natural aqueous outflow process in step 1303 allows relief of intraocular pressure, such that pressure is normalized and corresponding glaucoma symptoms may be relieved and progression of disease halted or slowed. Embodiments of the invention can be used to quantify a regional aqueous outflow with a level of detail not available in existing techniques. Therefore, embodiments can use such data to guide and target surgical or laser interventions to specific anatomical regions, for example, to maximize therapeutic effect and changes to aqueous outflow. For example, drilling laser drainage holes where the outflow is the lowest, resulting in the biggest therapeutic response.

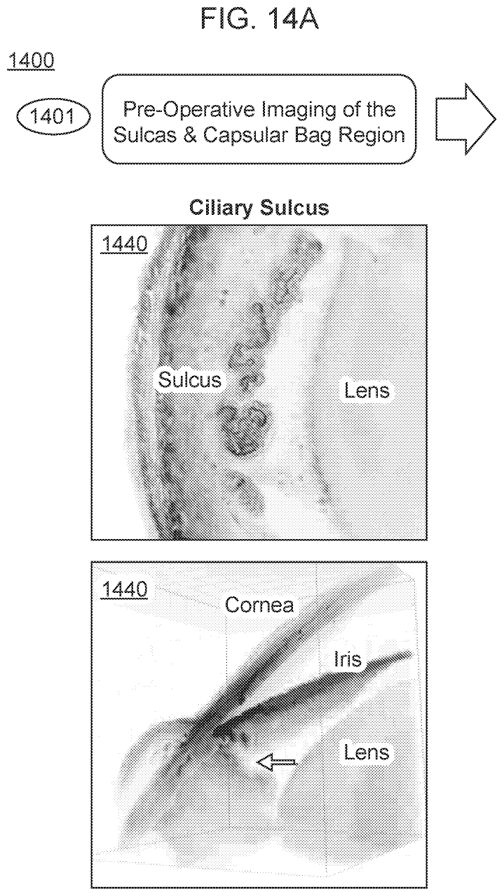

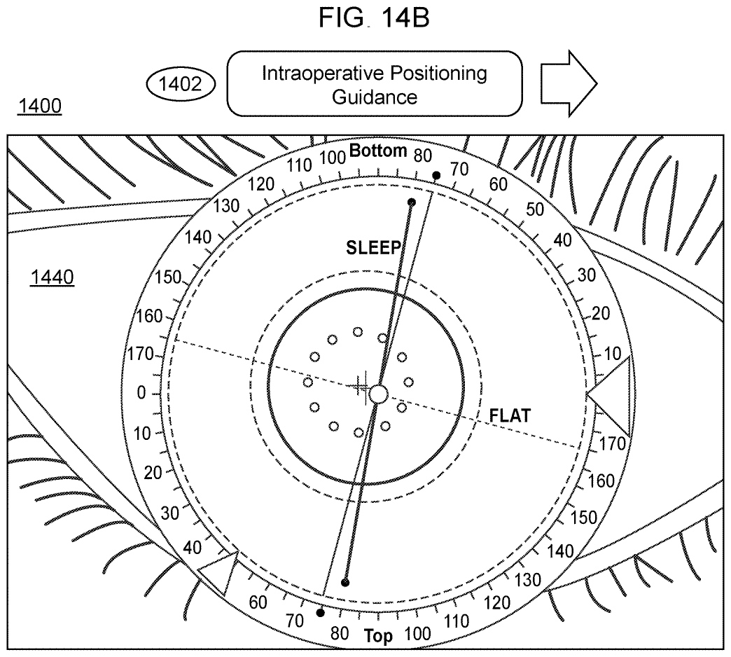

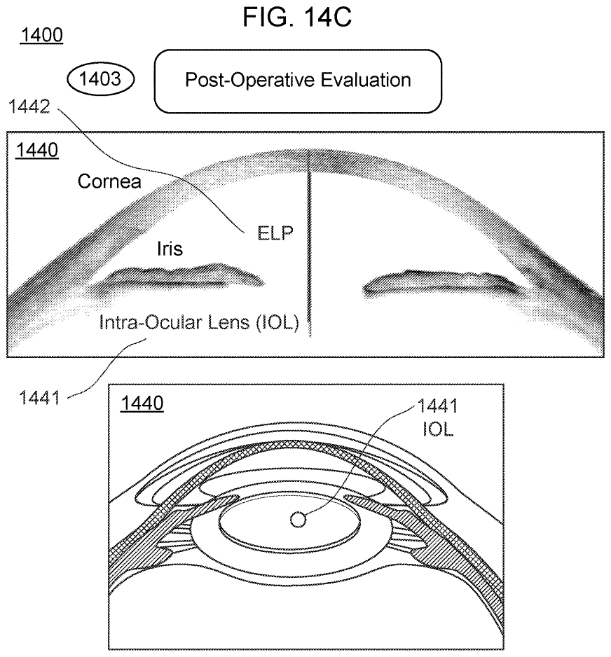

FIGS. 14A-C present an outline of another ophthalmic imaging and therapeutic application of embodiments of the present invention. In particular, FIGS. 14A-C depict steps of flow diagram 1400 for utilizing embodiments for imaging and optical manipulation for cataract lens planning, i.e., in connection with positioning, installing and evaluating an intraocular lens (IOL), according to an embodiment of the present invention.

Existing cataract surgery techniques are typically capable of achieving the desired refractive outcomes within +/−0.5 D at best. Such limitation is due to the inability to predict the final location of the intraocular lens (IOL), i.e., the effective lens position of the IOL. Utilizing embodiments of the present invention to image of one or more potential landing zones for an intraocular lens (IOL) and support structures thereof, i.e., haptics connected with the IOL, within ocular tissue 1440 enables a determination to be made of the correlation between the underlying anatomy of optical tissue 1440 and the post-surgery effective lens potion (ELP) of the intraocular lens (IOL), including haptics thereof, to further close the gap in achieving the desired refractive outcomes.

In step 1401 of FIG. 14A, pre-operative imaging of anatomy relevant to a cataract lens procedure, in particular positioning of an intraocular lens (IOL), such as, the sulcas and capsular bag region of ocular tissue 1440, are obtained utilizing imaging techniques according to the present invention. Such imaging in step 1401 identifies ocular and/or periocular anatomy within non-transparent tissue 1440 relevant to a cataract lens procedure such as the cornea, iris, lens and, in particular with respect to a cataract lens procedure involving an intraocular lens (IOL), such as ciliary sulcus and capsular bag region.

From step 1401 in FIG. 14A, flow diagram 1400 next moves to step 1402 in FIG. 14B.

In step 1402 in FIG. 14B, intraoperative imaging is applied to the relevant anatomical structures visualized in step 1401 present within non-transparent tissue 1440 to provide intraoperative positioning guidance with respect to positioning of an intraocular lens (IOL) including the lens and haptics thereof. An embodiment of the present invention comprising an optical system for imaging ocular tissue is used to image the anatomical structures identified in step 1401 and analyze the relevant features and orientations thereof the better position aspects of the IOL.

From step 1402 in FIG. 14B, flow diagram 1400 next moves to step 1403 in FIG. 14C.

Step 1403 in FIG. 14C entails post-operative evaluation of position of an implanted intraocular lens (IOL) 1441 and an evaluation of the effective lens position (ELP) of lens 1441.

Certain embodiments comprise performing at least a subset of the steps presented in FIGS. 14A-B after a lens is already in place. That is, embodiments may apply thermal laser treatment to the anatomy to modify the position of an existing lens (e.g., to shrink tissue to move the lens slightly forward or backward, thereby changing its effective power and focus position to the retina). Such embodiments provide an approach for making small corrections to aspects of an implanted lens in cases where the lens selection is off.

Other embodiments employ laser therapy to relevant anatomy including, in some cases, the sclera, to return reading/focusing ability to the eye (which is lost later in life). Certain such embodiments apply laser therapy to the ciliary body muscle and adjacent structure to “soften” these stiffened tissue structures to return some compliance/flexibility and allow return of accommodation.

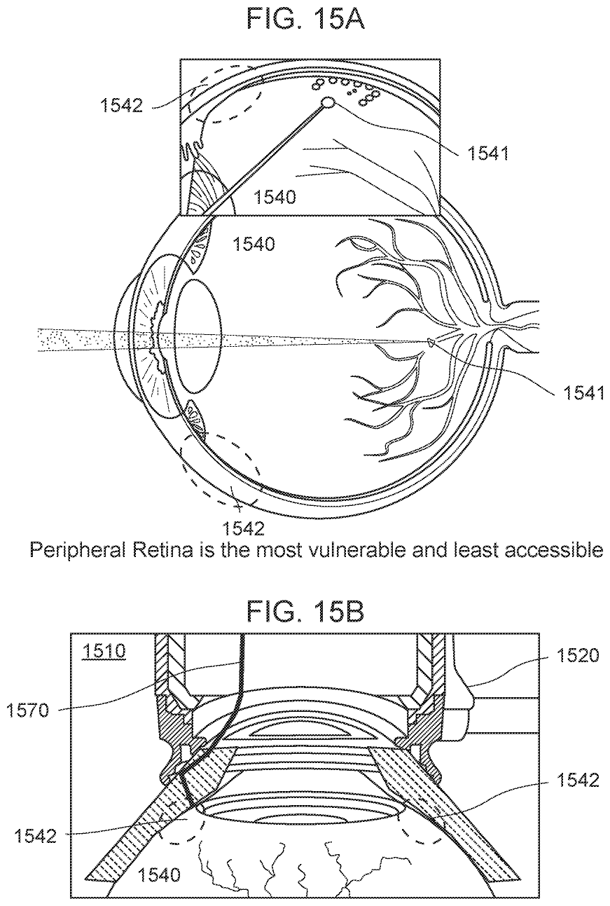

FIGS. 15A-B present schematics of another ophthalmic imaging and therapeutic application of embodiments of the present invention. In particular, FIGS. 15A-B depict utilizing embodiments for transscleral imaging and treatment, i.e., in connection with repairing or mitigating a retinal break, according to an embodiment of the present invention.

FIG. 15A depicts existing techniques in which laser light is focused onto ocular tissue 1540 to repair retinal break 1541. However, peripheral retina 1542 is the most vulnerable tissue with respect to retinal breaks but is also the least accessible for repair using existing techniques, as is highlighted in the close up within FIG. 15A.

FIG. 15B depicts utilizing an embodiment of an optical system of the present invention for use in repairing retinal breaks. FIG. 15B depicts applying an embodiment of the present invention with optical system 1510 with at least some treatment light routed through element 1570 to non-transparent ocular tissue 1540 in such a way as to access peripheral retina 1542 through non-transparent ocular tissue 1540. In embodiments, treatment light may also be applied, in part or in total, through an objective of optical system 1510 (i.e., an objective used for imaging). Optical system 1510 may be used to image as well as manipulate retinal break 1542, including, for example, repairing retinal break by using certain treatment light 1570 for providing therapy, such as providing photocoagulation therapy to retinal break 1542. Unlike existing techniques shown in FIG. 15A, the embodiment shown in FIG. 15B is well suited for high-resolution imaging and treatment of retinal breaks since it is not limited to accessing a retinal break 1541 through transparent tissue but instead can access a retinal break through non-transparent tissue, e.g., transscleral repair of retinal break 1541.

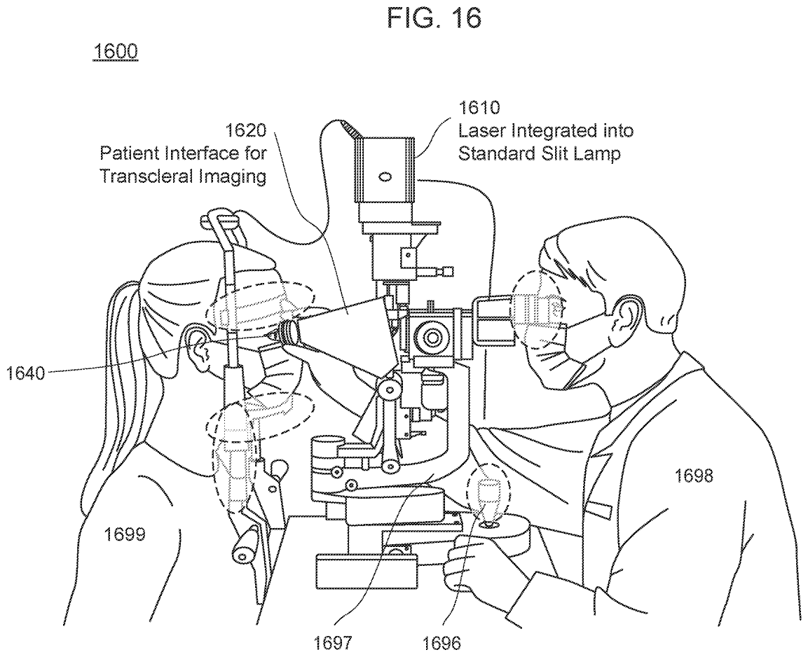

FIG. 16 depicts an overview of a potential structure 1600 for use with embodiments of the present invention in connection imaging ocular tissue 1640 of subject 1699. Optical system 1610 and adaptor 1620, in each case, according to an embodiment of the invention, may be affixed to supporting structure 1697 such that clinician 1698 can visualize optical tissue 1640 in real time, including while using controls 1696 to manipulate a focus of optical system 1610 such that different aspects of ocular tissue 1640 may be visualized.

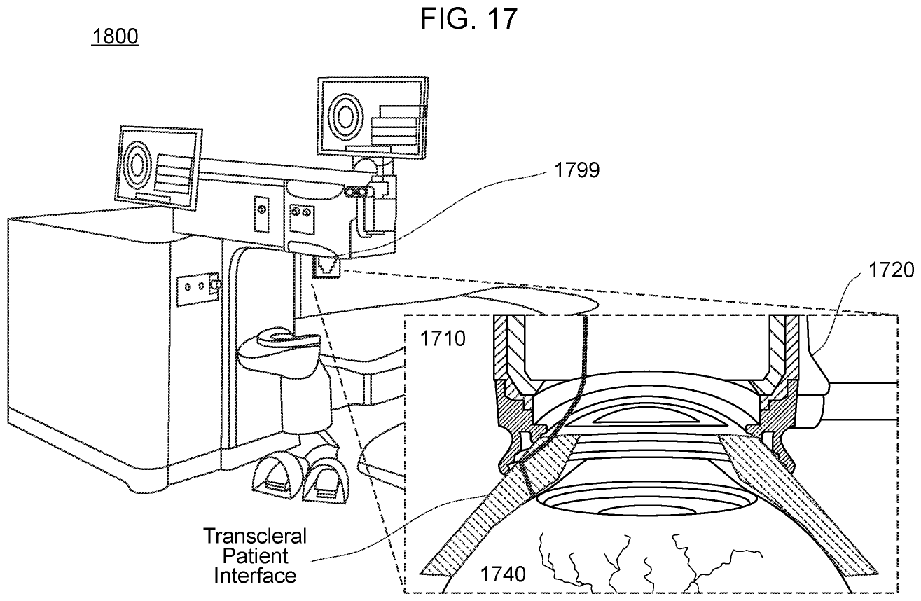

FIG. 17 depicts an overview of a potential structure 1700 for use with embodiments of the present invention in connection imaging ocular tissue 1740. Optical system 1710 and adaptor 1720, in each case, according to an embodiment of the invention, may be integrated into structure 1700 at location 1799 such that a subject can be positioned under location 1799 allowing access such that adaptor 1720 can interface with ocular tissue 1740. Optical system 1710 transmits light through element 1770 to non-transparent ocular or periocular tissue 1740 such that a structure present therein can be imaged or manipulated as described herein.

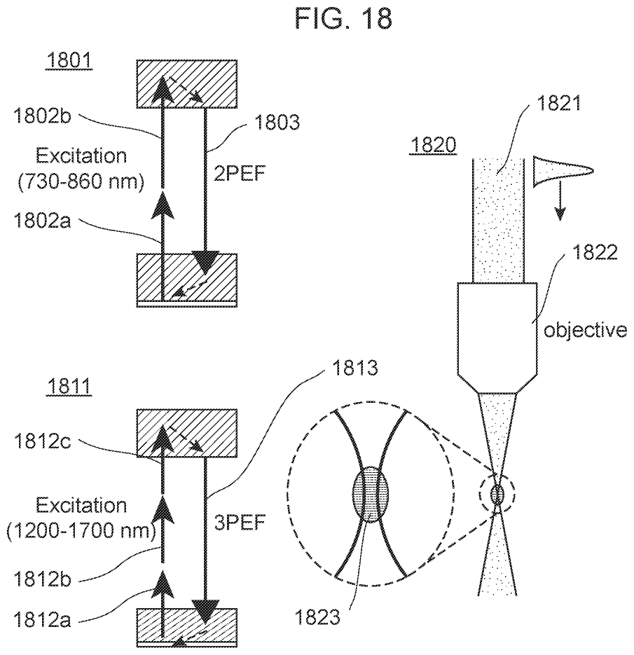

FIG. 18 depicts aspects of three-photon excited fluorescence (3PEF) versus two-photon excited fluorescence (2PEF). In 2PEF excitation 1801, excitation light comprising two photons, first and second photons 1802a, 1802b, with wavelengths between 600-1,800 nm are “combined” resulting in new photon 1803. The arrow representing new photon 1803 is shown as “shorter” than the sum of the lengths of the arrows representing photons 1802a, 1802b corresponding to aspects of a valence electron diagram and indicating that an amount of energy of photons 1802a, 1802b is not contributed to new photon 1803 but is lost due to heat emission. In 3PEF excitation 1811, excitation light comprising three photons, first, second and third photons 1812a, 1812b, 1812c with wavelengths between 800-3,500 nm are “combined” resulting in new photon 1813. The arrow representing new photon 1813 is shown as “shorter” than the sum of the lengths of the arrows representing photons 1812a, 1812b, 1812c corresponding to aspects of a valence electron diagram and indicating that an amount of energy of photons 1812a, 1812b, 1812c is not contributed to new photon 1813 but is lost due to heat emission. In embodiments utilizing second harmonic generation (SHG) or third harmonic generation (THG), energy is not lost to heat emissions (as indicated in FIG. 18); i.e., a length of an arrow representing a new photon would be of the same length as the arrows of photons “combined” to generate the new photon, in embodiments utilizing SHG or THG. While 3PEF and 2PEF are described in connection with FIG. 18, embodiments of the present invention are not so limited. For example, embodiments of the present invention further comprise other higher order processes, such as, for example, four photon excitation. Other contrast mechanisms used in embodiments comprise second harmonic generation (also a two-photon process like 2PEF) and third harmonic generation (also a three photon process like 3PEF). Aspects of optical system 1820 according to an embodiment of the invention are used to focus excitation light 1821 to focal point 1823 using objective lens 1822. Excitation light may be any convenient light capable of resulting in three-photon excited fluorescence (3PEF) or two-photon excited fluorescence (2PEF) or second harmonic generation (SHG) or third harmonic generation (THG) and may comprise, for example, pulsed light. In some cases, excitation light may comprise light with wavelength between 600-1,800 nm in connection with two-photon excited fluorescence (2PEF) or light with wavelength between 800-3,500 nm in connection with three-photon excited fluorescence (3PEF). Optical system 1820 is configured such that excitation light 1821 is focused using objective 1822 such that three photons can be “combined” (as shown in 3PEF illustration 1811) such that three-photon excited fluorescence (3PEF) occurs at focal point 1823. Aspects of the present invention comprise multiphoton laser therapy (i.e., energy deposition for burning or damage), which can also comprise multiphoton processes. Certain such embodiments need not collect image signal.

As described herein, embodiments of the present invention comprise imaging utilizing three-photon excited fluorescence (3PEF) as well as two-photon excited fluorescence (2PEF). In some cases, imaging a structure using two-photon excited fluorescence (2PEF) can be further enhanced by utilizing second harmonic generation (SHG) information. Similarly, in some cases, imaging a structure using three-photon excited fluorescence (3PEF) can be further enhanced by utilizing third harmonic generation (THG) information. Further details, including regarding two-photon excited fluorescence (2PEF), second harmonic generation (SHG), three-photon excited fluorescence (3PEF) and third harmonic generation (THG) are found in Zipfel W R, Williams R M, Webb W W. Nonlinear magic: multiphoton microscopy in the biosciences. Nat Biotechnol. 2003 Nov; 21(11): 1369-77. doi: 10.1038/nbt899. PMID: 14595365, the disclosure of which is herein incorporated in its entirety. Certain embodiments may further comprise still higher order processes, such as, for example, four-photon excited fluorescence (4PEF) or fourth harmonic generation (FHG).

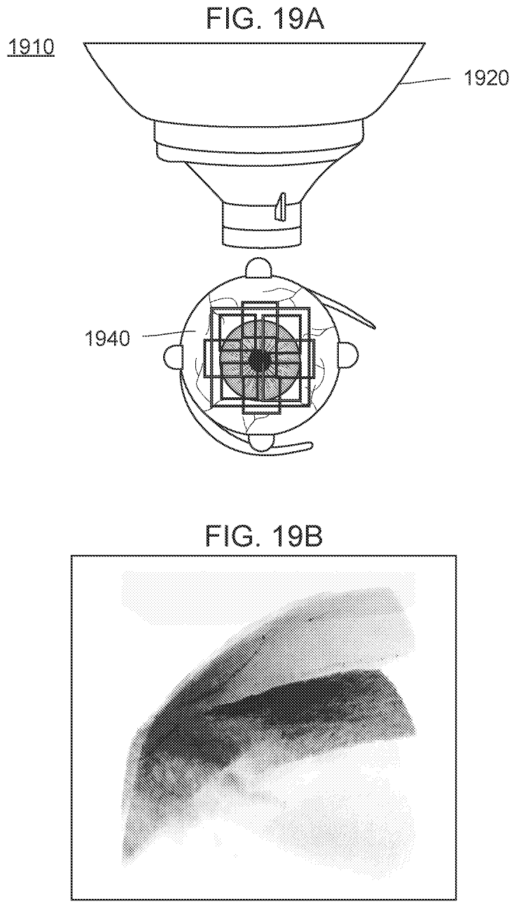

FIGS. 19A-B present an overview of another ophthalmic imaging and therapeutic application of embodiments of the present invention. In particular, FIGS. 19A-B depict measuring one or more angles, sulcus and zonular insertion. By “angle,” it is meant, in embodiments, the anterior chamber angle, i.e., the anatomical region where the iris and cornea meet, and where the trabecular meshwork is located and where aqueous drainage leaves the anterior chamber. By “zonular insertion,” it is meant, in embodiments, where the zonules fibers are positioned and attach (these fibers suspend the intraocular lens insert into their anchor points along the ciliary body). Using existing techniques, it is not possible to image or measure such aspects, which therefore leaves some approximation and/or guesswork in calculating the position of an intraocular lens. Measuring such aspects exactly (i.e., using embodiments of the present invention) would enable more precisely calculating the position of an intraocular lens implant after surgery, and significantly lower the errors intrinsic to calculation using existing techniques.

FIG. 19A depicts optical system 1910 according to an embodiment of the present invention being used with adaptor 1920 to interface with optical tissue 1940 for visualizing anatomical aspects of optical tissue 1940, for example, at certain insertion zones corresponding to different areas of the surface of ocular tissue 2040. FIG. 19B depicts imaging results from using optical system 1910 and adaptor 1940 of embodiments of the present invention in connection with imaging relevant anatomical structures. FIG. 19B shows a three-dimensional projection of the anterior segment demonstrating the cornea with corneal nerves, anterior chamber, and the iris.

Notwithstanding the appended claims, the disclosure is also defined by the following clauses:

1. A method of imaging a structure through non-transparent tissue, the method comprising:
deploying an excitation source to transmit light energy to a structure through non-transparent tissue;
detecting light emitted from the structure via multiphoton excitation through the non-transparent tissue; and
imaging the structure based on the detected light.

2. A method of manipulating a structure through non-transparent tissue, the method comprising:
deploying an excitation source to transmit light energy to a structure through non-transparent tissue; and
manipulating the structure using multiphoton excitation via light energy transmitted to the structure through the non-transparent tissue.

3. The method of any of the previous clauses, wherein the non-transparent tissue comprises non-transparent ocular or periocular tissue.

4. The method of any of the previous clauses, wherein the non-transparent tissue comprises one or more of: scleral tissue, retinal pigment epithelium (RPE), uvea, conjunctiva, Tenon's capsule, ocular muscles, ciliary body, palpebral conjunctiva, orbital septum, capsolupalpebral fascia, tarsus, tarsal glands, periocular adipose tissue or dermis.

5. The method of any of the previous clauses, wherein the non-transparent tissue comprises light-scattering tissue or light-absorbing tissue.

6. The method of any of the previous clauses, wherein the non-transparent tissue comprises pigmented uveal tissues.

7. The method of any of the previous clauses, wherein the structure comprises one or more of: scleral tissue, corneal tissue, ocular vasculature, suprachoroidal space, choroid, chorioretinal vasculature, retinal pigment epithelium (RPE), photoreceptors, conjunctiva, Tenon's capsule, ocular muscles, ciliary body, peripheral retina, orbital fat, palpebral tissues, dermatologic tissue, optionally, comprising one or more of epidermis, dermis or hypodermis, a tear duct, sulcas, capsular bag region, extraocular muscle, collagen, skin collagen, vascular tissue.

8. The method of any of the previous clauses, wherein the structure comprises one or more of: palpebral conjunctiva, orbital septum, capsolupalpebral fascia, tarsus, tarsal glands, periocular adipose tissue or dermis, capillaries, venules, veins, arterioles or arteries, optionally, of the choroid, circulating cells.

9. The method of any of the previous clauses, wherein the structure is present in front of (relative to the excitation source) a retinal pigment epithelium (RPE).

10. The method of any of clauses 1 to 8, wherein the structure is present behind (relative to the excitation source) a retinal pigment epithelium (RPE).

11. The method of any of the previous clauses, wherein the structure comprises light-scattering tissue.

12. The method of any of the previous clauses, wherein the structure comprises light-absorbing tissue.

13. The method of any of the previous clauses, wherein the method further comprises introducing fluorescent dye into the structure.

14. The method of any of the previous clauses, wherein the method further comprises labeling blood plasma present in the structure with a fluorescent dye.

15. The method of any of the previous clauses, further comprising imaging the structure over a specified volume.

16. The method of clause 15, wherein imaging the structure over a specified volume comprises gathering imaging data at a specified spatial resolution.

17. The method of any of the previous clauses, further comprising imaging the structure over a specified period of time.

18. The method of clause 17, wherein imaging the structure over a specified period of time comprises gathering imaging data at a specified pulse repetition rate.

19. The method of clause 18, wherein the pulse repetition rate is between Hz to 1,000 MHz.

20. The method of clause 19, wherein the pulse repetition rate is between kHz to 1,000 KHz.

21. The method of any of the previous clauses, wherein the method is a method of imaging cellular dynamics or imaging neuronal activity or imaging a corneal nerve or imaging aspects of a central nervus system.

22. The method of any of the previous clauses, wherein the method further comprises utilizing an invasive probe to image aspects of a central nervus system, optionally, further comprising thinning a region of a skull to image aspects of a central nervus system.

23. The method of any of the previous clauses, wherein the method further comprises visualizing nerve structures or pain receptors and, optionally, utilizing imaged nerve structures or pain receptors to mitigate pain.

24. The method of any of the previous clauses, wherein the method is a method of imaging neuronal activity, optionally with calcium indicators; or a method of imaging hemodynamics; or a method of imaging blood flow within micro-vasculature.

25. The method of any of the previous clauses, wherein the excitation source comprises one or more laser systems.

26. The method of any of the previous clauses, wherein the excitation source comprises one or more laser systems configured to emit light energy with average power output great than 100 Watts.

27. The method any of the previous clauses, wherein the excitation source comprises one or more laser systems that utilize dispersion compensation.

28. The method of any of the previous clauses, wherein the excitation source comprises one or more laser systems comprise a usable power band of 350 nm to 5,000 nm.

29. The method of any of the previous clauses, wherein the excitation source comprises a first laser.

30. The method of clause 29, wherein the first laser is a solid-state laser.

31. The method of any of clauses 29 to 30, wherein the first laser is a fiber laser.

32. The method of any of clauses 29 to 31, wherein the first laser is an ytterbium-doped fiber laser.

33. The method of any of clauses 29 to 32, wherein the first laser is configured to generate light energy having a wavelength ranging from 350 nm to 5.0 μm, pulse duration of 1 to 1,000 femtoseconds and maximum power output of greater than 100 W.

34. The method of any of clauses 29 to 33, wherein the first laser is configured to emit light energy with average power output of greater than 100 Watts.

35. The method of any of clauses 29 to 34, wherein the first laser comprises dispersion compensation.

36. The method of any of the previous clauses, wherein the excitation source comprises a second laser.

37. The method of clause 36, wherein the second laser is a titanium-doped sapphire laser.

38. The method of any of clauses 36 to 37, wherein the second laser is a fixed 1,045 nm laser.

39. The method of any of clauses 36 to 38, wherein the second laser is configured to generate light energy having a maximum power output exceeding 100 W.

40. The method of any of clauses 36 to 39, wherein the second laser comprises a usable power band of 350 nm to 5,000 nm.

41. The method of clause 40, wherein the second laser comprises a usable power band of 690 to 970 nm.

42. The method of any of the previous clauses, wherein the excitation source comprises a third component.

43. The method of clause 42, wherein the third component comprises a 1.7 μm excitation source.

44. The method of any of clauses 42 to 43, wherein the third component comprises Raman shifting of 1.5 μm light in a large mode area photonic crystal rod.

45. The method of any of clauses 42 to 44, wherein the third component emits excitation wavelengths between 350 nm and 5 μm.

46. The method of clause 45, wherein the third component emits excitation wavelengths between 550 nm and 3.6 μm.

47. The method of any of the previous clauses, wherein deploying an excitation source comprises translating the excitation source over a predetermined area.

48. The method of any of the previous clauses, wherein deploying an excitation source comprises articulating the excitation source around the non-transparent tissue.

49. The method of any of the previous clauses, wherein the excitation source comprises a pulsed laser.

50. The method of clause 49, wherein the pulsed laser generates light energy having a pulse duration lasting from 1 to 1,000 femtoseconds.

51. The method of any of clauses 49 to 50, wherein the pulsed laser generates light energy having a pulse repetition rate ranging from 1 Hz to 1,000 MHz.

52. The method of clause 51, wherein the pulsed laser generates light energy having a pulse repetition rate ranging from 1 Hz to 65 MHz.

53. The method of any of clauses 49 to 52, wherein the pulsed laser generates light with average power output great than 100 Watts.

54. The method of any of the previous clauses, wherein deploying an excitation source comprises deploying one or more of the following optical components: a laser scanner, a pulse compressor, a power attenuator or adaptive optics for wavefront shaping.

55. The method of any of the previous clauses, wherein light emitted from the structure via multiphoton excitation comprises stimulated light.

56. The method of clause 55, wherein stimulated light is excited via a higher-order nonlinear process.

57. The method of clause 56, wherein the higher-order nonlinear process comprises one or more of: two-photon excited fluorescence (2PEF) or second harmonic generation (SHG) or three-photon excited fluorescence (3PEF) or third harmonic generation (THG).

58. The method of any of the previous clauses, wherein light emitted via multiphoton excitation comprises light emitted from endogenous fluorophores present in the structure or from exogenous fluorophores present in the structure.

59. The method of any of the previous clauses, wherein endogenous or exogenous fluorophores present in the structure emit one or more of ultra-violet, blue, green, red or far red light.

60. The method of any of the previous clauses, wherein light emitted from the structure comprises light emitted via two-photon excitation (2PEF) and further comprises a second harmonic generation (SHG) signal.

61. The method of any of the previous clauses, wherein light emitted from the structure comprises light emitted via three-photon excitation (3PEF) and further comprises a third harmonic generation (THG) signal.

62. The method of any of the previous clauses, wherein deploying an excitation source to transmit light energy to a structure comprises spatially guiding the excitation source to transmit light energy through the non-transparent tissue.

63. The method of clause 62, wherein spatially guiding the excitation source to transmit light energy to the structure comprises using a multi-modal laser scanner to guide the excitation source.

64. The method of clause 63, wherein the multi-modal laser scanner is switchable between resonant imaging and patterned point-scanning.

65. The method of any of the previous clauses, further comprising employing an adaptive optics technique.

66. The method of any of the previous clauses, further comprising employing an adaptive optics technique applied to light transmitted from the excitation source.

67. The method of any of the previous clauses, further comprising employing an adaptive optics technique configured to increase efficiency of multiphoton excitation.

68. The method of any of the previous clauses, further comprising employing an adaptive optics technique configured to improve a resolution of the imaged structure.

69. The method of any of the previous clauses, further comprising employing an adaptive optics technique comprising one or more of: direct sensing or direct wavefront sensing or correction of wavefront distortions or an image point-spread function or a laser guide star technique or indirect (algorithmic) adaptive optics techniques, wherein indirect adaptive optics techniques optionally comprise determining an optimal excitation wavefront.

70. The method of any of the previous clauses, further comprising employing an adaptive optics technique comprising direct wavefront sensing comprising employing a Shack-Hartman sensor and a deformable mirror.

71. The method of any of the previous clauses, further comprising employing an adaptive optics technique configured to improve spatiotemporal resolution.

72. The method of any of the previous clauses, further comprising employing velocity analysis by particle tracking and cross-correlation analysis.

73. The method of any of the previous clauses, further comprising employing velocity analysis by particle tracking and cross-correlation analysis comprising measuring blood flow, wherein measuring blood flow, optionally, comprises measuring choroid blood flow.

74. The method of any of the previous clauses, further comprising velocity analysis by particle tracking and cross-correlation analysis comprising employing line-scanning particle image velocimetry (LS-PIV).

75. The method of any of the previous clauses, further comprising utilizing an imaged structure to perform flow analysis.

76. The method of any of the previous clauses, further comprising performing flow analysis, wherein the flow analysis comprises analysis of fluid flow within the structure.

77. The method of any of the previous clauses, comprising utilizing multi-core processors or parallel processing units to perform flow analysis, wherein the multi-core processors or parallel processing units optionally comprise graphics processing units (GPUs).

78. The method of any of the previous clauses, further comprising performing flow analysis substantially in real time.

79. The method of any of the previous clauses, further comprising performing flow analysis, wherein the flow analysis comprises generating a velocity map of fluid flow within the structure.

80. The method of any of the previous clauses, further comprising deploying sensors configured to collect back-scattered light emitted from the structure via multiphoton excitation through the non-transparent tissue.

81. The method of any of the previous clauses, further comprising deploying sensors configured to collect light other than back-scattered light emitted from the structure via multiphoton excitation through the non-transparent tissue.

82. The method of clause 81, wherein collecting light other than back-scattered light comprises collecting light emitted deep within the tissue.

83. The method of any of clauses 81 to 82, wherein collecting light other than back-scattered light comprises positioning a sensor directly over a cornea of an eye to capture emitted light from inside the eye.

84. The method of any of the previous clauses, further comprising imaging at subcellular resolution.

85. The method of any of the previous clauses, further comprising using imaging of the structure to detect or diagnose disease; or to detect or diagnose cancer or cancerous tissue; or to detect or diagnose melanoma; or to distinguish between cancerous and non-cancerous tissues; or to distinguish between cancerous and non-cancerous cells.

86. The method of any of the previous clauses, further comprising manipulating an imaged structure.

87. The method of any of the previous clauses, further comprising manipulating an imaged structure comprising using the excitation source to manipulate the structure.

88. The method of any of the previous clauses, further comprising manipulating the structure, wherein manipulating the structure comprises using the excitation source for one or more of: non-incisional therapy; or photo-tissue interactions, wherein, optionally, the photo-tissue interactions comprise laser-tissue interactions or laser-tissue perturbations; or multi-photon-mediated thermal damage; or non-thermal treatment; or photo-disruption; or blood vessel coagulation; or ablation.

89. The method of any of the previous clauses, wherein manipulating a structure, optionally, by using the excitation source for photo-tissue interactions, comprises providing treatment of tumors or cancerous tissue or cancerous cells.

90. The method of any of the previous clauses, wherein manipulating a structure, optionally, by using the excitation source for photo-tissue interactions, comprises configuring the excitation source for photo-disruption; or configuring the excitation source for non-thermal damage; or configuring the excitation source for multi-photon-mediated non-thermal damage; or configuring the excitation source for blood vessel coagulation; or configuring the excitation source to cause blood vessel coagulation; or configuring the excitation source to disrupt blood vessel coagulation.

91. The method of any of the previous clauses, wherein manipulating an imaged structure comprises ablating the structure; or using the excitation source to treat glaucoma; or using the excitation source to reduce aqueous production of ocular tissue; or damaging ciliary body to reduce aqueous production, optionally, by thermally damaging ciliary body or applying a photo disruption mediated process.

92. The method of any of the previous clauses, further comprising using the excitation source to treat glaucoma, optionally, comprising increasing outflow of aqueous humor.

93. The method of clause 92, wherein using the excitation source to increase outflow of aqueous humor comprises performing laser trabeculotomy through non-transparent tissue of an eye.

94. The method of clause 92, wherein using the excitation source to increase outflow of aqueous humor comprises performing laser trabeculoplasty directly through the non-transparent tissue of the eye.

95. The method of any of the previous clauses, further comprising manipulating the imaged structure using the excitation source to prevent or treat retinal breaks, wherein the retinal break is, optionally, a peripheral retinal break.

96. The method of any of the previous clauses, further comprising using the excitation source for non-incisional therapy comprising using the excitation source to prevent or treat retinal breaks, wherein the retinal break is, optionally, a peripheral retinal break.

97. The method of any of the previous clauses, further comprising preventing or treating retinal breaks comprising transscleral imaging or transscleral treatment.

98. The method of any of the previous clauses, further comprising preventing or treating retinal breaks comprising identifying and providing photocoagulation therapy to the retina.

99. The method of any of the previous clauses, further comprising using the excitation source for non-incisional therapy comprising: providing photocoagulation and thermal treatment, optionally, to tumors or cancerous tissue or cancerous cells or to ciliary body tumors or to peripheral choroidal tumors; or providing non-thermal treatment, optionally, to tumors or cancerous tissue or cancerous cells or to ciliary body tumors or to peripheral choroidal tumors; or.

100. The method of any of the previous clauses, further comprising manipulating the imaged structure using the excitation source to optically cross-link scleral tissue.

101. The method of any of the previous clauses, wherein the method is a method for prevention of myopia.

102. The method of any of any of the previous clauses, further comprising manipulating the imaged structure using the excitation source to visualize extraocular muscle function.

103. The method of any of the previous clauses, further comprising performing targeted alternation of extraocular muscle function.

104. The method of any of any of the preceding clauses, further comprising visualizing orbital fat.

105. The method of any of the preceding clauses, further comprising performing targeted thermal or photocoagulation therapy of orbital fat.

106. The method of any of the preceding clauses, further comprising visualizing palpebral tissues.

107. The method of any of the preceding clauses, further comprising manipulating the imaged structure by performing targeted alternation of palpebral tissues.

108. The method of any of the preceding clauses, further comprising using the excitation source to manipulate the structure employing an adaptive optics technique.

109. The method of any of the preceding clauses, further comprising using the excitation source to manipulate the structure employing an adaptive optics technique configured to increase efficiency of manipulating the imaged structure using the excitation source.

110. The method of any of the preceding clauses, further comprising using the excitation source to manipulate the structure employing an adaptive optics technique configured to improve an accuracy of the excitation source.

111. The method of any of the preceding clauses, further comprising using the excitation source to manipulate the structure employing an adaptive optics technique configured to improve the accuracy of the excitation source with respect to an aspect of the structure.

112. The method of any of the preceding clauses, further comprising using the excitation source to manipulate the structure employing an adaptive optics technique configured to improve the accuracy of the manipulation of the imaged structure.

113. The method of any of the preceding clauses, further comprising using the excitation source to manipulate the structure employing an adaptive optics technique, wherein the adaptive optics technique comprises one or more of: direct sensing or direct wavefront sensing or correction of wavefront distortions or an image point-spread function or a laser guide star technique, wherein direct wavefront sensing, optionally, comprises employing a Shack-Hartman sensor and a deformable mirror.

114. The method of any of the preceding clauses, further comprising using the excitation source to manipulate the structure employing an adaptive optics technique, wherein the adaptive optics technique is configured to improve spatiotemporal resolution.

115. The method of any of the preceding clauses, further comprising using the excitation source to manipulate dermatologic tissue, optionally, comprising one or more of: epidermis, dermis or hypodermis.

116. The method of any of the preceding clauses, further comprising using the excitation source to manipulate a tear duct, optionally, to facilitate fluid flow within the tear duct.

117. The method of any of the preceding clauses, further comprising using the excitation source to manipulate the structure to mitigate ocular tissue redness.

118. The method of any of the preceding clauses, further comprising using the excitation source to manipulate the structure to deliver therapy to the structure.

119. The method of any of previous clauses, wherein the method is a method of guiding delivery of gene therapy.

120. The method of clause 119, further comprising introducing a specified agent into the structure.

121. The method of clause 120, wherein the specified agent comprises cells.

122. The method of clause 121, wherein the cells comprise stem cells or engineered cells.

123. The method of clause 120, wherein the specified agent comprises an active agent.

124. The method of clause 120, wherein the specified agent comprises a molecule.

125. The method of clause 120, wherein introducing a specified agent into the structure comprises introducing the specified agent into one or more of: subretinal space or suprachoroidal space or subchoroidal space or intravitreal space or the ciliary body or the stroma of the sclera.

126. The method of any of clauses 120 to 125, further comprising assessing an effect of the specified agent on the structure based on imaging the structure.

127. The method of any of the previous clauses, further comprising guiding positioning of an intraocular implant based on imaging the structure.

128. The method of any of the previous clauses, further comprising predicting effective placement of intraocular implant based on imaging the structure.

129. The method of any of the previous clauses, further comprising imaging one or more of sulcas or capsular bag region.

130. The method of any of the previous clauses, further comprising guiding positioning of an intraocular implant based on imaging the structure while implanting the intraocular implant.

131. The method of any of the previous clauses, further comprising evaluating a position of an implanted intraocular implant based on imaging the structure.

132. The method of any of the previous clauses, further comprising evaluating an effective lens position (ELP) of an implanted intraocular implant based on imaging the structure.

133. The method of any of clauses 127 to 132, wherein the intraocular implant is an intraocular lens (IOL).

134. The method of any of the previous clauses, wherein the method is a method of transscleral imaging; or trans-conjunctiva imaging; or trans-Tenon's capsule imaging; or extraocular muscle imaging; or imaging through external palpebral tissue, optionally, comprising imaging through one or more of: dermis or muscle or aponeurosis; or imaging through the internal palpebral tissue, optionally, comprising one or more of: conjunctiva or tarsus or Meibomian glands or muscle; or trans-orbital septum imaging; or trans-capsolupalpebral fascia imaging; or trans-tarsus fascia imaging; or trans-tarsal gland imaging; or trans-periocular adipose tissue imaging; or trans-dermal imaging; or imaging through pigmented uveal tissues.

135. The method of any of the previous clauses, wherein the method is a method of imaging through light-scattering tissue; or light-absorbing tissue.

136. The method of any of the previous clauses, wherein the method is a method of quantification of blood flow, optionally, comprising one or more of: choroid blood flow or retinal blood flow or ciliary body blood flow or uveal blood flow or conjunctival blood flow.

137. The method of any of the previous clauses, wherein the method is a method of deploying multi-photon excitation microscopy on non-transparent tissue, optionally comprising ocular or periocular tissue.

138. The method of any of the previous clauses, wherein the method is a method of deploying multi-photon excitation microscopy through non-transparent tissue, optionally comprising ocular or periocular tissue.

139. The method of any of the previous clauses, wherein the method is a method of deploying multi-photon excitation microscopy comprising one or more of: two-photon excited fluorescence (2PEF) or second harmonic generation (SHG) or three-photon excited fluorescence (3PEF) or third harmonic generation (THG).

140. The method of any of the previous clauses, wherein the method is a method of imaging living tissue, optionally comprising ocular or periocular tissue.

141. The method of any of the previous clauses, wherein the method is a method of imaging a subject.

142. The method of any of the previous clauses, wherein the method is a method of imaging a subject with one or more of: glaucoma, a retinal break, a choroidal tumor, myopia.

143. The method of any of the previous clauses, wherein the method is a method of imaging a human subject.

144. The method of any of the previous clauses, wherein the method is a method of delivering therapy.

145. The method of any of the previous clauses, wherein the method is a method of providing cosmetic treatment.

146. The method of any of the previous clauses, wherein the method is a method of providing cosmetic dermatologic treatment, optionally, comprising one or more of: providing cosmetic surgery; treating scars; scar removal; treating acne scars; removing acne scars; treating skin discoloration; treating skin discoloration disorders; removing birthmarks; removing port-wine stains; removing tattoos; treating rosacea; removing hair; disrupting hair follicles; stimulating hair follicles; ablating hair follicle tissue; skin tightening.

147. The method of any of the previous clauses, further comprising manipulating the structure by performing controlled cutting of tissue, wherein the tissue is, optionally, dermatologic tissue.

148. The method of any of the previous clauses, wherein the method is a method of performing a skin biopsy procedure.

149. The method of any of the previous clauses, further comprising manipulating the structure by disrupting cancerous tissue or one or more cancer cells or ablating cancerous tissue or ablating one or more cancer cells or ablating melanoma or ablating skin melanoma.

150. The method of any of the previous clauses, wherein the method is a method of affecting tissue shape, optionally, comprising the shape of one or more fat deposits; or comprising reducing a volume of one or more fat deposits; or reducing one or more subdural fat deposits.

151. The method of any of the previous clauses, wherein the method is a method of tissue sculpting; or treating dry eye syndrome; or applying thermal energy to the structure; pain management.

152. The method of any of the previous clauses, further comprising using the excitation source to perforate the structure, optionally, in connection with providing thermal treatment to tissue.

153. The method of any of the preceding clauses, further comprising deploying an adaptor for coupling an optical system to non-transparent tissue.

154. The method of any of the preceding clauses, further comprising deploying a system for imaging a structure through non-transparent tissue.

155. The method of clause 154, wherein the system comprises:
an optical system configured to image a structure through non-transparent tissue;
an adaptor configured to couple the optical system to the non-transparent tissue;
a processor comprising memory operably coupled to the processor, wherein the memory comprises instructions stored thereon, which, when executed by the processor, cause the processor to:
   instruct the optical system to image a structure through the non-transparent tissue;
   receive information from the optical system about light emitted from the structure; and
   combine information about light emitted from the structure to generate an image the structure; and
an operable connection between the processor and the optical system.

156. An adaptor for coupling an optical system to non-transparent tissue, the adaptor comprising:
a first component configured to interface with an optical system configured to image or manipulate a structure through non-transparent tissue; and
a second component connected to the first component and configured to interface with non-transparent tissue.

157. The adaptor of clause 156, wherein the non-transparent tissue comprises ocular or periocular tissue.

158. The adaptor of clause 156, further comprising a coupling agent.

159. The adaptor of clause 158, wherein the coupling agent comprises a transparent media,
wherein the transparent media is optionally configured to increase a stability or duration of using the optical system, and the transparent media is optionally configured for use with imaging at longer wavelengths;
wherein the transparent media optionally comprises one or more of: water or a viscous gel optionally comprising sodium hyaluronate (optionally, comprising molecular weight: between 100,000 and 20,000,000 Daltons) or chondroitin sulfate (optionally, comprising molecular weight between 1,000 and 1,000,000 Daltons).

160. The adaptor of any of clauses 156 to 159, wherein the coupling agent is present between the optical system and the tissue.

161. The adaptor of clause 160, wherein the coupling agent is present between the first component and the tissue.

162. The adaptor of any of clauses 156 to 161, further comprising an immersion media.

163. The adaptor of clause 162, wherein a lens that focuses light into the tissue is present within the immersion media.

164. The adaptor of clause 163, wherein the immersion media comprises one or more of: air, water oil or a gel.

165. The adaptor of clause 164, wherein the water is deuterium oxide (heavy water).

166. The adaptor of clause 165, wherein the gel is a viscoelastic gel.

167. The adaptor of clause 166, wherein the viscoelastic gel is constituted with deuterium oxide (heavy water).

168. The adaptor of any of clauses 156 to 167, wherein the second component is configured to interface with tissue using suction.

169. The adaptor of clause 168, wherein the second component comprises a suction mechanism for attaching the second component and the tissue.

170. The adaptor of any of clauses 156 to 169, wherein the second component comprises a central portion.

171. The adaptor of clause 170, wherein the central portion is hollow.

172. The adaptor of clause 171, wherein the hollow central portion receives fluid.

173. The adaptor of clause 170, wherein the central portion is solid.

174. The adaptor of clause 173, wherein the solid central portion is optically transparent.

175. The adaptor of any of clauses 156 to 174, wherein the second component comprises an interface surface, wherein the interface surface contacts the non-transparent tissue.

176. The adaptor of clause 175, wherein the interface surface interfaces with ocular tissue.

177. The adaptor of clause 176, wherein the interface surface is shaped to contact ocular tissue.

178. The adaptor of clause 177, wherein the interface surface is shaped to be displaced relative to the cornea or sclerocorneal limbus.

179. The adaptor of any of clauses 176 to 178, wherein the interface surface is shaped to accommodate placement on a section of a cornea or sclerocorneal limbus.

180. The adaptor of any of clauses 176 to 179, wherein a section of the interface surface is depressed to accommodate placement on a cornea or sclerocorneal limbus.

181. The adaptor of any of clauses 156 to 180, wherein the second component interfaces with conjunctival fornix.

182. The adaptor of clause 181, wherein the second component comprises a shape to interface with the conjunctival fornix.

183. The adaptor of any of clauses 156 to 182, wherein the second component comprises a shape to fit between an eye and a lower eyelid of the eye.

184. The adaptor of any of clauses 156 to 183, wherein the second component comprises a shape to fit between an eye and an upper eyelid of the eye.

185. The adaptor of any of clauses 156 to 184, wherein the second component comprises a contact lens interface.

186. The adaptor of any of clauses 156 to 185, wherein the first component translates relative to the non-transparent ocular or periocular tissue.

187. The adaptor of any of clauses 156 to 185, wherein the first component allows the optical system to translate relative to the second component.

188. The adaptor of any of clauses 156 to 187, wherein the first component rotates relative to the non-transparent tissue.

189. The adaptor of any of clauses 156 to 188, wherein the first component allows the optical system to rotate relative to the non-transparent tissue.

190. The adaptor of any of clauses 156 to 189, wherein the first component articulates relative to the non-transparent tissue.

191. The adaptor of any of clauses 156 to 190, wherein the first component allows the optical system to articulate relative to the non-transparent tissue.

192. The adaptor of any of clauses 156 to 191, wherein the adaptor further comprises a mechanism to control translation or rotation or articulation of the optical system relative to the non-transparent tissue.

193. The adaptor of any of clauses 156 to 192, configured to perform any of the methods of clauses 1 to 155.

194. An immersion media for biological imaging comprising an immersion gel.

195. The immersion gel of clause 194, wherein the immersion gel comprises a hyaluronan gel.

196. The immersion gel of any of clauses 194 to 195, wherein the immersion gel comprises hyaluronic acid and deuterium oxide (heavy water).

197. The immersion gel of any of clauses 194 to 196, wherein the immersion gel is configured for imaging deep biological structures.

198. The immersion gel of any of clauses 194 to 197, wherein the immersion gel is configured for use with long wavelength lasers.

199. The immersion gel of clause 198, wherein the long wavelength laser emits light with wavelength 1,700 nm or greater.

200. The immersion gel of any of clauses 194 to 199, wherein the immersion gel is an adaptor between an optical system and biological tissue.

201. The method of any of clauses 1 to 155, further comprising employing an immersion gel according to any of clauses 194 to 199.

202. The adaptor of any of clauses 156 to 193, further comprising an immersion gel according to any of clauses 194 to 199.

203. A system for imaging a structure through non-transparent tissue, the system comprising:
an optical system configured to image a structure through non-transparent tissue;
an adaptor configured to couple the optical system to the non-transparent tissue according to any of clauses 156 to 193;
a processor comprising memory operably coupled to the processor, wherein the memory comprises instructions stored thereon, which, when executed by the processor, cause the processor to:
   instruct the optical system to image a structure through the non-transparent tissue;
   receive information from the optical system about light emitted from the structure; and
   combine information about light emitted from the structure to generate an image the structure; and
an operable connection between the processor and the optical system.

204. A system for manipulating a structure through non-transparent tissue, the system comprising:
an optical system configured to manipulate a structure through non-transparent tissue;
an adaptor configured to couple the optical system to the non-transparent tissue according to any of clauses 156 to 193;
a processor comprising memory operably coupled to the processor, wherein the memory comprises instructions stored thereon, which, when executed by the processor, cause the processor to: instruct the optical system to manipulate the structure through the non-transparent tissue; and
an operable connection between the processor and the optical system.

205. The system of any of clauses 203 to 204, wherein the non-transparent tissue comprises ocular or periocular tissue.

206. The system of any of clauses 203 to 205, wherein the optical system comprises a multi-photon excitation microscopy system.

207. The system of clause 206, wherein the multi-photon excitation microscopy system comprises:
an excitation source to emit light energy; and
a detector to sense light emitted from the structure via multiphoton excitation.

208. The system of clause 207, wherein the detector collects light that is backscattered.

209. The system of clause 208, wherein the detector collects light other than light that is backscattered.

210. The system of clause 209, wherein the detector collects light emitted deep within the tissue.

211. The system of clauses 207 to 210, wherein the detector is positioned directly over the cornea.

212. The system of any of clauses 203 to 211, wherein the optical system comprises a laser scanner.

213. The system of any of clauses 203 to 212, wherein the optical system comprises one or more of: a tube lens or a scan lens.

214. The system of any of clauses 203 to 213, wherein the optical system applies dispersion compensation.

215. The system of any of clauses 203 to 214, wherein the optical system applies adaptive optical correction.

216. The system of any of clauses 203 to 214, wherein the optical system applies power attenuation control.

217. The system of clause 216, wherein power attenuation control is applied by one or more of: an acousto-optic modulator or a Pockel Cell or paired and rotating polarizers.

218. The system of any of clauses 203 to 217, wherein the optical system comprises a high numerical aperture optic or objective lens.

219. The system of any of clauses 203 to 218, wherein the optical system comprises a translation stage.

220. The system of any of clauses 203 to 219, wherein the optical system employs optical scanning for adjusting focus.

221. The system of any of clauses 203 to 220, further comprising a mechanical component for adjusting focus in a Z-axis.

222. The system of clause 221, wherein the mechanical component comprises one or more of: a translation stage or a piezoelectric mechanism.

223. The system of any of clauses 203 to 222, wherein the optical system employs optical scanning for adjusting focus in a Z-axis.

224. The system of any of clauses 203 to 223, wherein the memory further comprises instructions, which, when executed by the processor, cause the processor to:
instruct the optical system to image a structure over a specified volume.

225. The system of any of clauses 203 to 224, wherein the memory further comprises instructions, which, when executed by the processor, cause the processor to:
instruct the optical system to translate or rotate or articulate relative to the non-transparent tissue.

226. The system of any of clauses 203 to 225, wherein the memory further comprises instructions, which, when executed by the processor, cause the processor to:
instruct the optical system to image a structure over a specified time period.

227. The system of any of clauses 203 to 226, wherein the memory further comprises instructions, which, when executed by the processor, cause the processor to:
instruct the optical system to manipulate the structure.

228. The system of any of clauses 203 to 227, wherein manipulating the imaged structure comprises using the excitation source for non-incisional therapy.

229. The system of clause 228, wherein the non-incisional therapy comprises one or more of: multi-photon-mediated thermal damage, photo-disruption, photo cross-linking, blood vessel coagulation, ablating the structure.

230. The system of any of clauses 203 to 229, wherein manipulating the structure comprises using the excitation source to treat glaucoma.

231. The system of clause 230, wherein using the excitation source to treat glaucoma comprises using the excitation source to reduce aqueous production of ocular tissue.

232. The system of clause 231, wherein using the excitation source to reduce aqueous production of ocular tissue comprises damaging ciliary body to reduce aqueous production.

233. The system of clause 232, wherein damaging ciliary body to reduce aqueous production comprises one or more of: thermally damaging ciliary body or applying a photo disruption mediated process.

234. The system of clause 233, wherein using the excitation source to treat glaucoma comprises using the excitation source to increase outflow of aqueous humor.

235. The system of clause 234, wherein using the excitation source to increase outflow of aqueous humor comprises performing laser trabeculotomy through the non-transparent tissue of the eye.

236. The system of clause 235, wherein performing laser trabeculoplasty comprises performing laser trabeculoplasty directly through the non-transparent tissue of the eye.

237. The system of any of clauses 203 to 236, wherein manipulating the imaged structure comprises using the excitation source to prevent or treat retinal breaks, wherein the retinal break is, optionally, a peripheral retinal break.

238. The system of clause 237, wherein preventing or treating retinal breaks comprises identifying and providing photocoagulation therapy to the retina.

239. The system of any of clauses 203 to 238, wherein manipulating the structure comprises providing photocoagulation and thermal treatment to ciliary body tumors or peripheral choroidal tumors.

240. The system of any of clauses 203 to 239, wherein manipulating the structure comprises optically cross-linking the scleral tissue for prevention of myopia.

241. The system of any of clauses 203 to 240, wherein manipulating the structure comprises visualizing and performing targeted alteration of extraocular muscle function.

242. The system of any of clauses 203 to 241, wherein manipulating the structure comprises visualizing and performing targeted thermal or photocoagulation therapy of the orbital fat.

243. The system of any of clauses 203 to 242, wherein manipulating the structure comprises visualizing and performing targeted alteration of palpebral tissues.

244. The system of any of clauses 203 to 243, wherein the processor comprises one or more multi-core processors or parallel processing units, wherein the multi-core processors or parallel processing units, optionally, comprise graphics processing units (GPUS).

245. The system of clause 244, wherein the multi-core processors or parallel processing units are configured to perform flow analysis, wherein, optionally, the flow analysis is performed substantially in real time.

246. The system of any of clauses 203 to 245, configured to perform any of the methods of clauses 1 to 155.

247. The system of any of clauses 203 to 246, further comprising an immersion gel according to any of clauses 194 to 199.

248. A kit for imaging or manipulating a structure through non-transparent tissue, comprising:
an adaptor according to any of clauses 156 to 193; and
packaging for the adaptor.

249. The kit according to clause 248, further comprising:
a coupling agent.

250. A kit comprising:
a coupling agent; and
packaging for the coupling agent.

251. The kit according to clause 248, further comprising:
an immersion media.

252. A kit comprising:
an immersion media; and
packaging for the immersion media.

253. A kit for imaging or manipulating a structure through non-transparent tissue, comprising:
a system according to any of clauses 203 to 247; and
packaging for the system.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of imaging a structure through non-transparent tissue, the method comprising:
deploying an excitation source to transmit light energy to a structure through non-transparent tissue;
detecting light emitted from the structure via multiphoton excitation through the non-transparent tissue; and
imaging the structure based on the detected light.

2. A method of manipulating a structure through non-transparent tissue, the method comprising:
deploying an excitation source to transmit light energy to a structure through non-transparent tissue; and
manipulating the structure using multiphoton excitation via light energy transmitted to the structure through the non-transparent tissue.

3. The method of any of the previous claims, wherein the non-transparent tissue comprises non-transparent ocular or periocular tissue.

4. The method of any of the previous claims, wherein the non-transparent tissue comprises one or more of: scleral tissue, retinal pigment epithelium (RPE), uvea, conjunctiva, Tenon's capsule, ocular muscles, ciliary body, palpebral conjunctiva, orbital septum, capsolupalpebral fascia, tarsus, tarsal glands, periocular adipose tissue or dermis.

5. The method of any of the previous claims, wherein the non-transparent tissue comprises light-scattering tissue or light-absorbing tissue.

6. The method of any of the previous claims, wherein the non-transparent tissue comprises pigmented uveal tissues.

7. The method of any of the previous claims, wherein the structure comprises one or more of: scleral tissue, corneal tissue, ocular vasculature, suprachoroidal space, choroid, chorioretinal vasculature, retinal pigment epithelium (RPE), photoreceptors, conjunctiva, Tenon's capsule, ocular muscles, ciliary body, peripheral retina, orbital fat, palpebral tissues, dermatologic tissue, optionally, comprising one or more of epidermis, dermis or hypodermis, a tear duct, sulcas, capsular bag region, extraocular muscle, collagen, skin collagen, vascular tissue.

8. The method of any of the previous claims, wherein the structure comprises one or more of: palpebral conjunctiva, orbital septum, capsolupalpebral fascia, tarsus, tarsal glands, periocular adipose tissue or dermis, capillaries, venules, veins, arterioles or arteries, optionally, of the choroid, circulating cells.

9. The method of any of the previous claims, wherein the structure is present in front of (relative to the excitation source) a retinal pigment epithelium (RPE).

10. The method of any of claims 1 to 8, wherein the structure is present behind (relative to the excitation source) a retinal pigment epithelium (RPE).

11. The method of any of the previous claims, wherein the structure comprises light-scattering tissue.

12. The method of any of the previous claims, wherein the structure comprises light-absorbing tissue.

13. The method of any of the previous claims, wherein the method further comprises introducing fluorescent dye into the structure.

14. The method of any of the previous claims, wherein the method further comprises labeling blood plasma present in the structure with a fluorescent dye.

15. The method of any of the previous claims, further comprising imaging the structure over a specified volume.

16. The method of claim 15, wherein imaging the structure over a specified volume comprises gathering imaging data at a specified spatial resolution.

17. The method of any of the previous claims, further comprising imaging the structure over a specified period of time.

18. The method of claim 17, wherein imaging the structure over a specified period of time comprises gathering imaging data at a specified pulse repetition rate.

19. The method of claim 18, wherein the pulse repetition rate is between 1 Hz to 1,000 MHz.

20. The method of claim 19, wherein the pulse repetition rate is between 1 kHz to 1,000 KHz.

21. The method of any of the previous claims, wherein the method is a method of imaging cellular dynamics or imaging neuronal activity or imaging a corneal nerve or imaging aspects of a central nervus system.

22. The method of any of the previous claims, wherein the method further comprises utilizing an invasive probe to image aspects of a central nervus system, optionally, further comprising thinning a region of a skull to image aspects of a central nervus system.

23. The method of any of the previous claims, wherein the method further comprises visualizing nerve structures or pain receptors and, optionally, utilizing imaged nerve structures or pain receptors to mitigate pain.

24. The method of any of the previous claims, wherein the method is a method of imaging neuronal activity, optionally with calcium indicators; or a method of imaging hemodynamics; or a method of imaging blood flow within micro-vasculature.

25. The method of any of the previous claims, wherein the excitation source comprises one or more laser systems.

26. The method of any of the previous claims, wherein the excitation source comprises one or more laser systems configured to emit light energy with average power output great than 100 Watts.

27. The method any of the previous claims, wherein the excitation source comprises one or more laser systems that utilize dispersion compensation.

28. The method of any of the previous claims, wherein the excitation source comprises one or more laser systems comprise a usable power band of 350 nm to 5,000 nm.

29. The method of any of the previous claims, wherein the excitation source comprises a first laser.

30. The method of claim 29, wherein the first laser is a solid-state laser.

31. The method of any of claims 29 to 30, wherein the first laser is a fiber laser.

32. The method of any of claims 29 to 31, wherein the first laser is an ytterbium-doped fiber laser.

33. The method of any of claims 29 to 32, wherein the first laser is configured to generate light energy having a wavelength ranging from 350 nm to 5.0 μm, pulse duration of 1 to 1,000 femtoseconds and maximum power output of greater than 100 W.

34. The method of any of claims 29 to 33, wherein the first laser is configured to emit light energy with average power output of greater than 100 Watts.

35. The method of any of claims 29 to 34, wherein the first laser comprises dispersion compensation.

36. The method of any of the previous claims, wherein the excitation source comprises a second laser.

37. The method of claim 36, wherein the second laser is a titanium-doped sapphire laser.

38. The method of any of claims 36 to 37, wherein the second laser is a fixed 1,045 nm laser.

39. The method of any of claims 36 to 38, wherein the second laser is configured to generate light energy having a maximum power output exceeding 100 W.

40. The method of any of claims 36 to 39, wherein the second laser comprises a usable power band of 350 nm to 5,000 nm.

41. The method of claim 40, wherein the second laser comprises a usable power band of 690 to 970 nm.

42. The method of any of the previous claims, wherein the excitation source comprises a third component.

43. The method of claim 42, wherein the third component comprises a 1.7 μm excitation source.

44. The method of any of claims 42 to 43, wherein the third component comprises Raman shifting of 1.5 μm light in a large mode area photonic crystal rod.

45. The method of any of claims 42 to 44, wherein the third component emits excitation wavelengths between 350 nm and 5 μm.

46. The method of claim 45, wherein the third component emits excitation wavelengths between 550 nm and 3.6 μm.

47. The method of any of the previous claims, wherein deploying an excitation source comprises translating the excitation source over a predetermined area.

48. The method of any of the previous claims, wherein deploying an excitation source comprises articulating the excitation source around the non-transparent tissue.

49. The method of any of the previous claims, wherein the excitation source comprises a pulsed laser.

50. The method of claim 49, wherein the pulsed laser generates light energy having a pulse duration lasting from 1 to 1,000 femtoseconds.

51. The method of any of claims 49 to 50, wherein the pulsed laser generates light energy having a pulse repetition rate ranging from 1 Hz to 1,000 MHz.

52. The method of claim 51, wherein the pulsed laser generates light energy having a pulse repetition rate ranging from 1 Hz to 65 MHz.

53. The method of any of claims 49 to 52, wherein the pulsed laser generates light with average power output great than 100 Watts.

54. The method of any of the previous claims, wherein deploying an excitation source comprises deploying one or more of the following optical components: a laser scanner, a pulse compressor, a power attenuator or adaptive optics for wavefront shaping.

55. The method of any of the previous claims, wherein light emitted from the structure via multiphoton excitation comprises stimulated light.

56. The method of claim 55, wherein stimulated light is excited via a higher-order nonlinear process.

57. The method of claim 56, wherein the higher-order nonlinear process comprises one or more of: two-photon excited fluorescence (2PEF) or second harmonic generation (SHG) or three-photon excited fluorescence (3PEF) or third harmonic generation (THG).

58. The method of any of the previous claims, wherein light emitted via multiphoton excitation comprises light emitted from endogenous fluorophores present in the structure or from exogenous fluorophores present in the structure.

59. The method of any of the previous claims, wherein endogenous or exogenous fluorophores present in the structure emit one or more of ultra-violet, blue, green, red or far red light.

60. The method of any of the previous claims, wherein light emitted from the structure comprises light emitted via two-photon excitation (2PEF) and further comprises a second harmonic generation (SHG) signal.

61. The method of any of the previous claims, wherein light emitted from the structure comprises light emitted via three-photon excitation (3PEF) and further comprises a third harmonic generation (THG) signal.

62. The method of any of the previous claims, wherein deploying an excitation source to transmit light energy to a structure comprises spatially guiding the excitation source to transmit light energy through the non-transparent tissue.

63. The method of claim 62, wherein spatially guiding the excitation source to transmit light energy to the structure comprises using a multi-modal laser scanner to guide the excitation source.

64. The method of claim 63, wherein the multi-modal laser scanner is switchable between resonant imaging and patterned point-scanning.

65. The method of any of the previous claims, further comprising employing an adaptive optics technique.

66. The method of any of the previous claims, further comprising employing an adaptive optics technique applied to light transmitted from the excitation source.

67. The method of any of the previous claims, further comprising employing an adaptive optics technique configured to increase efficiency of multiphoton excitation.

68. The method of any of the previous claims, further comprising employing an adaptive optics technique configured to improve a resolution of the imaged structure.

69. The method of any of the previous claims, further comprising employing an adaptive optics technique comprising one or more of: direct sensing or direct wavefront sensing or correction of wavefront distortions or an image point-spread function or a laser guide star technique or indirect (algorithmic) adaptive optics techniques, wherein indirect adaptive optics techniques optionally comprise determining an optimal excitation wavefront.

70. The method of any of the previous claims, further comprising employing an adaptive optics technique comprising direct wavefront sensing comprising employing a Shack-Hartman sensor and a deformable mirror.

71. The method of any of the previous claims, further comprising employing an adaptive optics technique configured to improve spatiotemporal resolution.

72. The method of any of the previous claims, further comprising employing velocity analysis by particle tracking and cross-correlation analysis.

73. The method of any of the previous claims, further comprising employing velocity analysis by particle tracking and cross-correlation analysis comprising measuring blood flow, wherein measuring blood flow, optionally, comprises measuring choroid blood flow.

74. The method of any of the previous claims, further comprising velocity analysis by particle tracking and cross-correlation analysis comprising employing line-scanning particle image velocimetry (LS-PIV).

75. The method of any of the previous claims, further comprising utilizing an imaged structure to perform flow analysis.

76. The method of any of the previous claims, further comprising performing flow analysis, wherein the flow analysis comprises analysis of fluid flow within the structure.

77. The method of any of the previous claims, comprising utilizing multi-core processors or parallel processing units to perform flow analysis, wherein the multi-core processors or parallel processing units optionally comprise graphics processing units (GPUS).

78. The method of any of the previous claims, further comprising performing flow analysis substantially in real time.

79. The method of any of the previous claims, further comprising performing flow analysis, wherein the flow analysis comprises generating a velocity map of fluid flow within the structure.

80. The method of any of the previous claims, further comprising deploying sensors configured to collect back-scattered light emitted from the structure via multiphoton excitation through the non-transparent tissue.

81. The method of any of the previous claims, further comprising deploying sensors configured to collect light other than back-scattered light emitted from the structure via multiphoton excitation through the non-transparent tissue.

82. The method of claim 81, wherein collecting light other than back-scattered light comprises collecting light emitted deep within the tissue.

83. The method of any of claims 81 to 82, wherein collecting light other than back-scattered light comprises positioning a sensor directly over a cornea of an eye to capture emitted light from inside the eye.

84. The method of any of the previous claims, further comprising imaging at subcellular resolution.

85. The method of any of the previous claims, further comprising using imaging of the structure to detect or diagnose disease; or to detect or diagnose cancer or cancerous tissue; or to detect or diagnose melanoma; or to distinguish between cancerous and non-cancerous tissues; or to distinguish between cancerous and non-cancerous cells.

86. The method of any of the previous claims, further comprising manipulating an imaged structure.

87. The method of any of the previous claims, further comprising manipulating an imaged structure comprising using the excitation source to manipulate the structure.

88. The method of any of the previous claims, further comprising manipulating the structure, wherein manipulating the structure comprises using the excitation source for one or more of: non-incisional therapy; or photo-tissue interactions, wherein, optionally, the photo-tissue interactions comprise laser-tissue interactions or laser-tissue perturbations; or multi-photon-mediated thermal damage; or non-thermal treatment; or photo-disruption; or blood vessel coagulation; or ablation.

89. The method of any of the previous claims, wherein manipulating a structure, optionally, by using the excitation source for photo-tissue interactions, comprises providing treatment of tumors or cancerous tissue or cancerous cells.

90. The method of any of the previous claims, wherein manipulating a structure, optionally, by using the excitation source for photo-tissue interactions, comprises configuring the excitation source for photo-disruption; or configuring the excitation source for non-thermal damage; or configuring the excitation source for multi-photon-mediated non-thermal damage; or configuring the excitation source for blood vessel coagulation; or configuring the excitation source to cause blood vessel coagulation; or configuring the excitation source to disrupt blood vessel coagulation.

91. The method of any of the previous claims, wherein manipulating an imaged structure comprises ablating the structure; or using the excitation source to treat glaucoma; or using the excitation source to reduce aqueous production of ocular tissue; or damaging ciliary body to reduce aqueous production, optionally, by thermally damaging ciliary body or applying a photo disruption mediated process.

92. The method of any of the previous claims, further comprising using the excitation source to treat glaucoma, optionally, comprising increasing outflow of aqueous humor.

93. The method of claim 92, wherein using the excitation source to increase outflow of aqueous humor comprises performing laser trabeculotomy through non-transparent tissue of an eye.

94. The method of claim 92, wherein using the excitation source to increase outflow of aqueous humor comprises performing laser trabeculoplasty directly through the non-transparent tissue of the eye.

95. The method of any of the previous claims, further comprising manipulating the imaged structure using the excitation source to prevent or treat retinal breaks, wherein the retinal break is, optionally, a peripheral retinal break.

96. The method of any of the previous claims, further comprising using the excitation source for non-incisional therapy comprising using the excitation source to prevent or treat retinal breaks, wherein the retinal break is, optionally, a peripheral retinal break.

97. The method of any of the previous claims, further comprising preventing or treating retinal breaks comprising transscleral imaging or transscleral treatment.

98. The method of any of the previous claims, further comprising preventing or treating retinal breaks comprising identifying and providing photocoagulation therapy to the retina.

99. The method of any of the previous claims, further comprising using the excitation source for non-incisional therapy comprising: providing photocoagulation and thermal treatment, optionally, to tumors or cancerous tissue or cancerous cells or to ciliary body tumors or to peripheral choroidal tumors; or providing non-thermal treatment, optionally, to tumors or cancerous tissue or cancerous cells or to ciliary body tumors or to peripheral choroidal tumors; or.

100. The method of any of the previous claims, further comprising manipulating the imaged structure using the excitation source to optically cross-link scleral tissue.

101. The method of any of the previous claims, wherein the method is a method for prevention of myopia.

102. The method of any of any of the previous claims, further comprising manipulating the imaged structure using the excitation source to visualize extraocular muscle function.

103. The method of any of the previous claims, further comprising performing targeted alternation of extraocular muscle function.

104. The method of any of any of the preceding claims, further comprising visualizing orbital fat.

105. The method of any of the preceding claims, further comprising performing targeted thermal or photocoagulation therapy of orbital fat.

106. The method of any of the preceding claims, further comprising visualizing palpebral tissues.

107. The method of any of the preceding claims, further comprising manipulating the imaged structure by performing targeted alternation of palpebral tissues.

108. The method of any of the preceding claims, further comprising using the excitation source to manipulate the structure employing an adaptive optics technique.

109. The method of any of the preceding claims, further comprising using the excitation source to manipulate the structure employing an adaptive optics technique configured to increase efficiency of manipulating the imaged structure using the excitation source.

110. The method of any of the preceding claims, further comprising using the excitation source to manipulate the structure employing an adaptive optics technique configured to improve an accuracy of the excitation source.

111. The method of any of the preceding claims, further comprising using the excitation source to manipulate the structure employing an adaptive optics technique configured to improve the accuracy of the excitation source with respect to an aspect of the structure.

112. The method of any of the preceding claims, further comprising using the excitation source to manipulate the structure employing an adaptive optics technique configured to improve the accuracy of the manipulation of the imaged structure.

113. The method of any of the preceding claims, further comprising using the excitation source to manipulate the structure employing an adaptive optics technique, wherein the adaptive optics technique comprises one or more of: direct sensing or direct wavefront sensing or correction of wavefront distortions or an image point-spread function or a laser guide star technique, wherein direct wavefront sensing, optionally, comprises employing a Shack-Hartman sensor and a deformable mirror.

114. The method of any of the preceding claims, further comprising using the excitation source to manipulate the structure employing an adaptive optics technique, wherein the adaptive optics technique is configured to improve spatiotemporal resolution.

115. The method of any of the preceding claims, further comprising using the excitation source to manipulate dermatologic tissue, optionally, comprising one or more of: epidermis, dermis or hypodermis.

116. The method of any of the preceding claims, further comprising using the excitation source to manipulate a tear duct, optionally, to facilitate fluid flow within the tear duct.

117. The method of any of the preceding claims, further comprising using the excitation source to manipulate the structure to mitigate ocular tissue redness.

118. The method of any of the preceding claims, further comprising using the excitation source to manipulate the structure to deliver therapy to the structure.

119. The method of any of previous claims, wherein the method is a method of guiding delivery of gene therapy.

120. The method of claim 119, further comprising introducing a specified agent into the structure.

121. The method of claim 120, wherein the specified agent comprises cells.

122. The method of claim 121, wherein the cells comprise stem cells or engineered cells.

123. The method of claim 120, wherein the specified agent comprises an active agent.

124. The method of claim 120, wherein the specified agent comprises a molecule.

125. The method of claim 120, wherein introducing a specified agent into the structure comprises introducing the specified agent into one or more of: subretinal space or suprachoroidal space or subchoroidal space or intravitreal space or the ciliary body or the stroma of the sclera.

126. The method of any of claims 120 to 125, further comprising assessing an effect of the specified agent on the structure based on imaging the structure.

127. The method of any of the previous claims, further comprising guiding positioning of an intraocular implant based on imaging the structure.

128. The method of any of the previous claims, further comprising predicting effective placement of intraocular implant based on imaging the structure.

129. The method of any of the previous claims, further comprising imaging one or more of sulcas or capsular bag region.

130. The method of any of the previous claims, further comprising guiding positioning of an intraocular implant based on imaging the structure while implanting the intraocular implant.

131. The method of any of the previous claims, further comprising evaluating a position of an implanted intraocular implant based on imaging the structure.

132. The method of any of the previous claims, further comprising evaluating an effective lens position (ELP) of an implanted intraocular implant based on imaging the structure.

133. The method of any of claims 127 to 132, wherein the intraocular implant is an intraocular lens (IOL).

134. The method of any of the previous claims, wherein the method is a method of transscleral imaging; or trans-conjunctiva imaging; or trans-Tenon's capsule imaging; or extraocular muscle imaging; or imaging through external palpebral tissue, optionally, comprising imaging through one or more of: dermis or muscle or aponeurosis; or imaging through the internal palpebral tissue, optionally, comprising one or more of: conjunctiva or tarsus or Meibomian glands or muscle; or trans-orbital septum imaging; or trans-capsolupalpebral fascia imaging; or trans-tarsus fascia imaging; or trans-tarsal gland imaging; or trans-periocular adipose tissue imaging; or trans-dermal imaging; or imaging through pigmented uveal tissues.

135. The method of any of the previous claims, wherein the method is a method of imaging through light-scattering tissue; or light-absorbing tissue.

136. The method of any of the previous claims, wherein the method is a method of quantification of blood flow, optionally, comprising one or more of: choroid blood flow or retinal blood flow or ciliary body blood flow or uveal blood flow or conjunctival blood flow.

137. The method of any of the previous claims, wherein the method is a method of deploying multi-photon excitation microscopy on non-transparent tissue, optionally comprising ocular or periocular tissue.

138. The method of any of the previous claims, wherein the method is a method of deploying multi-photon excitation microscopy through non-transparent tissue, optionally comprising ocular or periocular tissue.

139. The method of any of the previous claims, wherein the method is a method of deploying multi-photon excitation microscopy comprising one or more of: two-photon excited fluorescence (2PEF) or second harmonic generation (SHG) or three-photon excited fluorescence (3PEF) or third harmonic generation (THG).

140. The method of any of the previous claims, wherein the method is a method of imaging living tissue, optionally comprising ocular or periocular tissue.

141. The method of any of the previous claims, wherein the method is a method of imaging a subject.

142. The method of any of the previous claims, wherein the method is a method of imaging a subject with one or more of: glaucoma, a retinal break, a choroidal tumor, myopia.

143. The method of any of the previous claims, wherein the method is a method of imaging a human subject.

144. The method of any of the previous claims, wherein the method is a method of delivering therapy.

145. The method of any of the previous claims, wherein the method is a method of providing cosmetic treatment.

146. The method of any of the previous claims, wherein the method is a method of providing cosmetic dermatologic treatment, optionally, comprising one or more of: providing cosmetic surgery; treating scars; scar removal; treating acne scars; removing acne scars; treating skin discoloration; treating skin discoloration disorders; removing birthmarks; removing port-wine stains; removing tattoos; treating rosacea; removing hair; disrupting hair follicles; stimulating hair follicles; ablating hair follicle tissue; skin tightening.

147. The method of any of the previous claims, further comprising manipulating the structure by performing controlled cutting of tissue, wherein the tissue is, optionally, dermatologic tissue.

148. The method of any of the previous claims, wherein the method is a method of performing a skin biopsy procedure.

149. The method of any of the previous claims, further comprising manipulating the structure by disrupting cancerous tissue or one or more cancer cells or ablating cancerous tissue or ablating one or more cancer cells or ablating melanoma or ablating skin melanoma.

150. The method of any of the previous claims, wherein the method is a method of affecting tissue shape, optionally, comprising the shape of one or more fat deposits; or comprising reducing a volume of one or more fat deposits; or reducing one or more subdural fat deposits.

151. The method of any of the previous claims, wherein the method is a method of tissue sculpting; or treating dry eye syndrome; or applying thermal energy to the structure; pain management.

152. The method of any of the previous claims, further comprising using the excitation source to perforate the structure, optionally, in connection with providing thermal treatment to tissue.

153. The method of any of the preceding claims, further comprising deploying an adaptor for coupling an optical system to non-transparent tissue.

154. The method of any of the preceding claims, further comprising deploying a system for imaging a structure through non-transparent tissue.

155. The method of claim 154, wherein the system comprises:
an optical system configured to image a structure through non-transparent tissue;
an adaptor configured to couple the optical system to the non-transparent tissue;
a processor comprising memory operably coupled to the processor, wherein the memory comprises instructions stored thereon, which, when executed by the processor, cause the processor to:
instruct the optical system to image a structure through the non-transparent tissue;
receive information from the optical system about light emitted from the structure; and
combine information about light emitted from the structure to generate an image the structure; and
an operable connection between the processor and the optical system.

156. An adaptor for coupling an optical system to non-transparent tissue, the adaptor comprising:
a first component configured to interface with an optical system configured to image or manipulate a structure through non-transparent tissue; and
a second component connected to the first component and configured to interface with non-transparent tissue.

157. The adaptor of claim 156, wherein the non-transparent tissue comprises ocular or periocular tissue.

158. The adaptor of claim 156, further comprising a coupling agent.

159. The adaptor of claim 158, wherein the coupling agent comprises a transparent media,
wherein the transparent media is optionally configured to increase a stability or duration of using the optical system, and the transparent media is optionally configured for use with imaging at longer wavelengths;
wherein the transparent media optionally comprises one or more of: water or a viscous gel optionally comprising sodium hyaluronate (optionally, comprising molecular weight: between 100,000 and 20,000,000 Daltons) or chondroitin sulfate (optionally, comprising molecular weight between 1,000 and 1,000,000 Daltons).

160. The adaptor of any of claims 156 to 159, wherein the coupling agent is present between the optical system and the tissue.

161. The adaptor of claim 160, wherein the coupling agent is present between the first component and the tissue.

162. The adaptor of any of claims 156 to 161, further comprising an immersion media.

163. The adaptor of claim 162, wherein a lens that focuses light into the tissue is present within the immersion media.

164. The adaptor of claim 163, wherein the immersion media comprises one or more of: air, water oil or a gel.

165. The adaptor of claim 164, wherein the water is deuterium oxide (heavy water).

166. The adaptor of claim 165, wherein the gel is a viscoelastic gel.

167. The adaptor of claim 166, wherein the viscoelastic gel is constituted with deuterium oxide (heavy water).

168. The adaptor of any of claims 156 to 167, wherein the second component is configured to interface with tissue using suction.

169. The adaptor of claim 168, wherein the second component comprises a suction mechanism for attaching the second component and the tissue.

170. The adaptor of any of claims 156 to 169, wherein the second component comprises a central portion.

171. The adaptor of claim 170, wherein the central portion is hollow.

172. The adaptor of claim 171, wherein the hollow central portion receives fluid.

173. The adaptor of claim 170, wherein the central portion is solid.

174. The adaptor of claim 173, wherein the solid central portion is optically transparent.

175. The adaptor of any of claims 156 to 174, wherein the second component comprises an interface surface, wherein the interface surface contacts the non-transparent tissue.

176. The adaptor of claim 175, wherein the interface surface interfaces with ocular tissue.

177. The adaptor of claim 176, wherein the interface surface is shaped to contact ocular tissue.

178. The adaptor of claim 177, wherein the interface surface is shaped to be displaced relative to the cornea or sclerocorneal limbus.

179. The adaptor of any of claims 176 to 178, wherein the interface surface is shaped to accommodate placement on a section of a cornea or sclerocorneal limbus.

180. The adaptor of any of claims 176 to 179, wherein a section of the interface surface is depressed to accommodate placement on a cornea or sclerocorneal limbus.

181. The adaptor of any of claims 156 to 180, wherein the second component interfaces with conjunctival fornix.

182. The adaptor of claim 181, wherein the second component comprises a shape to interface with the conjunctival fornix.

183. The adaptor of any of claims 156 to 182, wherein the second component comprises a shape to fit between an eye and a lower eyelid of the eye.

184. The adaptor of any of claims 156 to 183, wherein the second component comprises a shape to fit between an eye and an upper eyelid of the eye.

185. The adaptor of any of claims 156 to 184, wherein the second component comprises a contact lens interface.

186. The adaptor of any of claims 156 to 185, wherein the first component translates relative to the non-transparent ocular or periocular tissue.

187. The adaptor of any of claims 156 to 185, wherein the first component allows the optical system to translate relative to the second component.

188. The adaptor of any of claims 156 to 187, wherein the first component rotates relative to the non-transparent tissue.

189. The adaptor of any of claims 156 to 188, wherein the first component allows the optical system to rotate relative to the non-transparent tissue.

190. The adaptor of any of claims 156 to 189, wherein the first component articulates relative to the non-transparent tissue.

191. The adaptor of any of claims 156 to 190, wherein the first component allows the optical system to articulate relative to the non-transparent tissue.

192. The adaptor of any of claims 156 to 191, wherein the adaptor further comprises a mechanism to control translation or rotation or articulation of the optical system relative to the non-transparent tissue.

193. The adaptor of any of claims 156 to 192, configured to perform any of the methods of claims 1 to 155.

194. An immersion media for biological imaging comprising an immersion gel.

195. The immersion gel of claim 194, wherein the immersion gel comprises a hyaluronan gel.

196. The immersion gel of any of claims 194 to 195, wherein the immersion gel comprises hyaluronic acid and deuterium oxide (heavy water).

197. The immersion gel of any of claims 194 to 196, wherein the immersion gel is configured for imaging deep biological structures.

198. The immersion gel of any of claims 194 to 197, wherein the immersion gel is configured for use with long wavelength lasers.

199. The immersion gel of claim 198, wherein the long wavelength laser emits light with wavelength 1,700 nm or greater.

200. The immersion gel of any of claims 194 to 199, wherein the immersion gel is an adaptor between an optical system and biological tissue.

201. The method of any of claims 1 to 155, further comprising employing an immersion gel according to any of claims 194 to 199.

202. The adaptor of any of claims 156 to 193, further comprising an immersion gel according to any of claims 194 to 199.

203. A system for imaging a structure through non-transparent tissue, the system comprising:
an optical system configured to image a structure through non-transparent tissue;
an adaptor configured to couple the optical system to the non-transparent tissue according to any of claims 156 to 193;
a processor comprising memory operably coupled to the processor, wherein the memory comprises instructions stored thereon, which, when executed by the processor, cause the processor to:
instruct the optical system to image a structure through the non-transparent tissue;
receive information from the optical system about light emitted from the structure; and
combine information about light emitted from the structure to generate an image the structure; and
an operable connection between the processor and the optical system.

204. A system for manipulating a structure through non-transparent tissue, the system comprising:
an optical system configured to manipulate a structure through non-transparent tissue;
an adaptor configured to couple the optical system to the non-transparent tissue according to any of claims 156 to 193;
a processor comprising memory operably coupled to the processor, wherein the memory comprises instructions stored thereon, which, when executed by the processor, cause the processor to: instruct the optical system to manipulate the structure through the non-transparent tissue; and
an operable connection between the processor and the optical system.

205. The system of any of claims 203 to 204, wherein the non-transparent tissue comprises ocular or periocular tissue.

206. The system of any of claims 203 to 205, wherein the optical system comprises a multi-photon excitation microscopy system.

207. The system of claim 206, wherein the multi-photon excitation microscopy system comprises:
an excitation source to emit light energy; and
a detector to sense light emitted from the structure via multiphoton excitation.

208. The system of claim 207, wherein the detector collects light that is backscattered.

209. The system of claim 208, wherein the detector collects light other than light that is backscattered.

210. The system of claim 209, wherein the detector collects light emitted deep within the tissue.

211. The system of claims 207 to 210, wherein the detector is positioned directly over the cornea.

212. The system of any of claims 203 to 211, wherein the optical system comprises a laser scanner.

213. The system of any of claims 203 to 212, wherein the optical system comprises one or more of: a tube lens or a scan lens.

214. The system of any of claims 203 to 213, wherein the optical system applies dispersion compensation.

215. The system of any of claims 203 to 214, wherein the optical system applies adaptive optical correction.

216. The system of any of claims 203 to 214, wherein the optical system applies power attenuation control.

217. The system of claim 216, wherein power attenuation control is applied by one or more of: an acousto-optic modulator or a Pockel Cell or paired and rotating polarizers.

218. The system of any of claims 203 to 217, wherein the optical system comprises a high numerical aperture optic or objective lens.

219. The system of any of claims 203 to 218, wherein the optical system comprises a translation stage.

220. The system of any of claims 203 to 219, wherein the optical system employs optical scanning for adjusting focus.

221. The system of any of claims 203 to 220, further comprising a mechanical component for adjusting focus in a Z-axis.

222. The system of claim 221, wherein the mechanical component comprises one or more of: a translation stage or a piezoelectric mechanism.

223. The system of any of claims 203 to 222, wherein the optical system employs optical scanning for adjusting focus in a Z-axis.

224. The system of any of claims 203 to 223, wherein the memory further comprises instructions, which, when executed by the processor, cause the processor to:
instruct the optical system to image a structure over a specified volume.

225. The system of any of claims 203 to 224, wherein the memory further comprises instructions, which, when executed by the processor, cause the processor to:
instruct the optical system to translate or rotate or articulate relative to the non-transparent tissue.

226. The system of any of claims 203 to 225, wherein the memory further comprises instructions, which, when executed by the processor, cause the processor to:
instruct the optical system to image a structure over a specified time period.

227. The system of any of claims 203 to 226, wherein the memory further comprises instructions, which, when executed by the processor, cause the processor to:
instruct the optical system to manipulate the structure.

228. The system of any of claims 203 to 227, wherein manipulating the imaged structure comprises using the excitation source for non-incisional therapy.

229. The system of claim 228, wherein the non-incisional therapy comprises one or more of: multi-photon-mediated thermal damage, photo-disruption, photo cross-linking, blood vessel coagulation, ablating the structure.

230. The system of any of claims 203 to 229, wherein manipulating the structure comprises using the excitation source to treat glaucoma.

231. The system of claim 230, wherein using the excitation source to treat glaucoma comprises using the excitation source to reduce aqueous production of ocular tissue.

232. The system of claim 231, wherein using the excitation source to reduce aqueous production of ocular tissue comprises damaging ciliary body to reduce aqueous production.

233. The system of claim 232, wherein damaging ciliary body to reduce aqueous production comprises one or more of: thermally damaging ciliary body or applying a photo disruption mediated process.

234. The system of claim 233, wherein using the excitation source to treat glaucoma comprises using the excitation source to increase outflow of aqueous humor.

235. The system of claim 234, wherein using the excitation source to increase outflow of aqueous humor comprises performing laser trabeculotomy through the non-transparent tissue of the eye.

236. The system of claim 235, wherein performing laser trabeculoplasty comprises performing laser trabeculoplasty directly through the non-transparent tissue of the eye.

237. The system of any of claims 203 to 236, wherein manipulating the imaged structure comprises using the excitation source to prevent or treat retinal breaks, wherein the retinal break is, optionally, a peripheral retinal break.

238. The system of claim 237, wherein preventing or treating retinal breaks comprises identifying and providing photocoagulation therapy to the retina.

239. The system of any of claims 203 to 238, wherein manipulating the structure comprises providing photocoagulation and thermal treatment to ciliary body tumors or peripheral choroidal tumors.

240. The system of any of claims 203 to 239, wherein manipulating the structure comprises optically cross-linking the scleral tissue for prevention of myopia.

241. The system of any of claims 203 to 240, wherein manipulating the structure comprises visualizing and performing targeted alteration of extraocular muscle function.

242. The system of any of claims 203 to 241, wherein manipulating the structure comprises visualizing and performing targeted thermal or photocoagulation therapy of the orbital fat.

243. The system of any of claims 203 to 242, wherein manipulating the structure comprises visualizing and performing targeted alteration of palpebral tissues.

244. The system of any of claims 203to 243, wherein the processor comprises one or more multi-core processors or parallel processing units, wherein the multi-core processors or parallel processing units, optionally, comprise graphics processing units (GPUS).

245. The system of claim 244, wherein the multi-core processors or parallel processing units are configured to perform flow analysis, wherein, optionally, the flow analysis is performed substantially in real time.

246. The system of any of claims 203 to 245, configured to perform any of the methods of claims 1 to 155.

247. The system of any of claims 203 to 246, further comprising an immersion gel according to any of claims 194 to 199.

248. A kit for imaging or manipulating a structure through non-transparent tissue, comprising:
an adaptor according to any of claims 156 to 193; and
packaging for the adaptor.

249. The kit according to claim 248, further comprising:
a coupling agent.

250. A kit comprising:
a coupling agent; and
packaging for the coupling agent.

251. The kit according to claim 248, further comprising:
an immersion media.

252. A kit comprising:
an immersion media; and
packaging for the immersion media.

253. A kit for imaging or manipulating a structure through non-transparent tissue, comprising:
a system according to any of claims 203 to 247; and
packaging for the system.
